(19) United States
(12) Patent Application Publication
(10) Pub. No.: US 2011/0112151 A1
(43) Pub. Date: May 12, 2011

(54) COMPOUND CAPABLE OF INHIBITING 11-BETA HYDROXYSTERIOD DEHYDROGENASE

(76) Inventors: Nigel Vicker, Berkshire (GB); Xiangdong Su, Berkshire (GB); Fabienne Pradaux-Caggiano, Berkshire (GB); Barry Victor Lioyd Potter, Berkshire (GB)

(21) Appl. No.: 12/919,707

(22) PCT Filed: Feb. 25, 2009
(86) PCT No.: PCT/GB2009/000518

§ 371 Date: Dec. 30, 2010

(30) Foreign Application Priority Data
Feb. 26, 2008 (GB) 0803494.4

(51) Int. Cl. A61K 31/44; A61K 31/4406; A61K 31/4402; A61K 31/4196; A61K 31/4184; A61K 31/4164; C07D 213/82; C07D 213/71; C07D 213/70; C07D 213/50; C07D 249/12; C07D 235/28; C07D 233/42; A61P 19/02; A61P 3/10; A61P 35/00; A61P 3/04; A61P 19/10; A61P 9/12; A61P 37/00; A61P 11/06

(52) U.S. Cl. 514/346; 514/277; 514/345; 514/347; 514/384; 514/395; 514/398; 546/291; 546/294; 546/302; 546/340; 548/264.4; 548/307.1; 548/325.1

(57) ABSTRACT

There is provided a compound of formula R₁—CO—X—Y—Z—R₂ wherein X and Z are each optional groups that are, independently, saturated or unsaturated carbon chains having a length of 1 to 3 carbons; Y is SO, S, SO₂, CH═CH, CH₂CH₂ or O; R₁ is

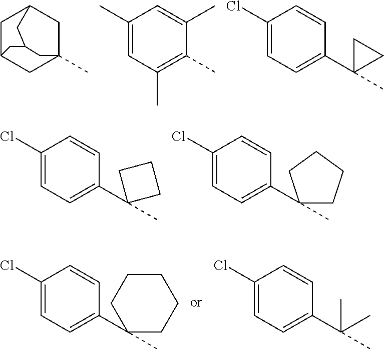

wherein [symbol] denotes the point of attachment; R₂ is a heteroaryl group comprising an optionally substituted 5 or 6 membered ring, which ring contains only carbon and at least one nitrogen, or contains only carbon, and at least two nitrogens and at least one sulfur; and wherein
(i) when R₁ is

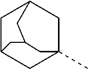

and —CO—X—Y—Z— is CO—CH₂—SO, CO—CH₂—S, or CO—CH₂—SO₂, R₂ is other than

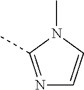

and; (ii) when R₁ is

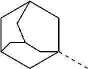

and —CO—X—Y—Z— is —CO—CH₂—O—, R₂ is other than

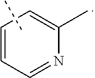

### FIELD OF INVENTION

The present invention relates to a compound. In particular the present invention provides compounds capable of inhibiting 11β-hydroxysteroid dehydrogenase (11β-HSD).

### INTRODUCTION

### The Role of Glucocorticoids

Glucocorticoids are synthesised in the adrenal cortex from cholesterol. The principle glucocorticoid in the human body is cortisol. This hormone is synthesised and secreted in response to the adrenocortictrophic hormone (ACTH) from the pituitary gland in a circadian, episodic manner, but the secretion of this hormone can also be stimulated by stress, exercise and infection. Cortisol circulates mainly bound to transcortin (cortisol binding protein) or albumin and only a small fraction is free (5-10%) for biological processes [1].

Cortisol has a wide range of physiological effects, including regulation of carbohydrate, protein and lipid metabolism, regulation of normal growth and development, influence on cognitive function, resistance to stress and mineralocorticoid activity. Cortisol works in the opposite direction compared to insulin meaning a stimulation of hepatic gluconeogenesis, inhibition of peripheral glucose uptake and increased blood glucose concentration. Glucocorticoids are also essential in the regulation of the immune response. When circulating at higher concentrations glucocorticoids are immunosuppressive and are used pharmacologically as anti-inflammatory agents.

Glucocorticoids like other steroid hormones are lipophilic and penetrate the cell membrane freely. Cortisol binds, primarily, to the intracellular glucocorticoid receptor (GR) that then acts as a transcription factor to induce the expression of glucocorticoid responsive genes, and as a result of that protein synthesis.

### The Role of the 11β-HSD Enzyme

The conversion of cortisol (F) to its inactive metabolite cortisone (E) by 11β-HSD was first described in the 1950's, however it was not until later that the biological importance for this conversion was suggested [2]. In 1983 Krozowski et al. showed that the mineralocorticoid receptor (MR) has equal binding affinities for glucocorticoids and mineralocorticoids [3]. Because the circulating concentration of cortisol is 100 times higher than that of aldosterone and during times of stress or high activity even more, it was not clear how the MR remained mineralocorticoid specific and was not constantly occupied by glucocorticoids. Earlier Ulick et al. [4] had described the hypertensive condition known as, “apparent mineralocorticoid excess” (AME), and observed that whilst secretion of aldosterone from the adrenals was in fact low the peripheral metabolism of cortisol was disrupted. These discoveries lead to the suggestion of a protective role for the enzymes. By converting cortisol to cortisone in mineralocorticoid dependent tissues 11β-HSD enzymes protect the MR from occupation by glucocorticoids and allow it to be mineralcorticoid specific. Aldosterone itself is protected from the enzyme by the presence of an aldehyde group at the C-18 position.

Congenital defects in the 11β-HSD enzyme results in over occupation of the MR by cortisol and hypertensive and hypokalemic symptoms seen in AME.

Localisation of the 11β-HSD showed that the enzyme and its activity is highly present in the MR dependent tissues, kidney and parotid. However in tissues where the MR is not mineralocorticoid specific and is normally occupied by glucocorticoids, 11 β-HSD is not present in these tissues, for example in the heart and hippocampus [5]. This research also showed that inhibition of 11 β-HSD caused a loss of the aldosterone specificity of the MR in these mineralocorticoid dependent tissues.

It has been shown that two iso-enzymes of 11 β-HSD exist. Both are members of the short chain alcohol dehydrogenase (SCAD) superfamily which have been widely conserved throughout evolution. 11 β-HSD type 2 acts as a dehydrogenase to convert the secondary alcohol group at the C-11 position of cortisol to a secondary ketone, so producing the less active metabolite cortisone. 11 β-HSD type 1 is thought to act mainly in vivo as a reductase, that is in the opposite direction to type 2 [6][see below]. 11 β-HSD type 1 and type 2 have only a 30% amino acid homology.

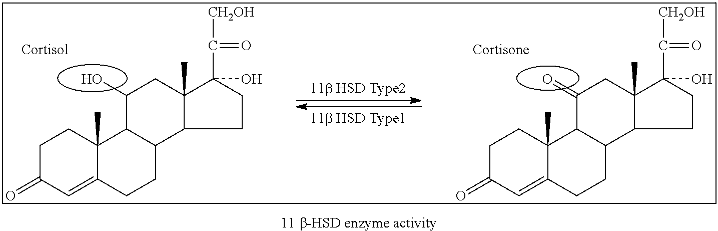

The intracellular activity of cortisol is dependent on the concentration of glucocorticoids and can be modified and independently controlled without involving the overall secretion and synthesis of the hormone.

### The Role of 11 β-HSD Type 1

The direction of 11 β-HSD type 1 reaction in vivo is generally accepted to be opposite to the dehydrogenation of type 2. In vivo homozygous mice with a disrupted type 1 gene are unable to convert cortisone to cortisol, giving further evidence for the reductive activity of the enzyme [7]. 11 β-HSD type 1 is expressed in many key glucocorticoid regulated tissues like the liver, pituitary, gonad, brain, adipose and adrenals; however, the function of the enzyme in many of these tissues is poorly understood [8].

The concentration of cortisone in the body is higher than that of cortisol. Cortisone also binds poorly to binding globulins, making cortisone many times more biologically available. Although cortisol is secreted by the adrenal cortex, there is a growing amount of evidence that the intracellular conversion of E to F may be an important mechanism in regulating the action of glucocorticoids [9].

It may be that 11 β-HSD type 1 allows certain tissues to convert cortisone to cortisol to increase local glucocorticoid activity and potentiate adaptive response and counteracting the type 2 activity that could result in a fall in active glucocorticoids [10]. Potentiation of the stress response would be especially important in the brain and high levels of 11 β-HSD type 1 are found around the hippocampus, further proving the role of the enzyme. 11 β-HSD type 1 also seems to play an important role in hepatocyte maturation [8].

Another emerging role of the 11 β-HSD type 1 enzyme is in the detoxification process of many non-steroidal carbonyl compounds. Reduction of the carbonyl group of many toxic compounds is a common way to increase solubility and therefore increase their excretion. The 11 β-HSD type 1 enzyme has recently been shown to be active in lung tissue [11]. Type 1 activity is not seen until after birth, therefore mothers who smoke during pregnancy expose their children to the harmful effects of tobacco before the child is able to metabolically detoxify this compound.

### The Role of 11 β-HSD Type 2

As already stated earlier the 11 β-HSD type 2 converts cortisol to cortisone, thus protecting the MR in many key regulatory tissues of the body. The importance of protecting the MR from occupation by glucocorticoids is seen in patients with AME or liquorice intoxification. Defects or inactivity of the type 2 enzyme results in hypertensive syndromes and research has shown that patients with an hypertensive syndrome have an increased urinary excretion ratio of cortisol:cortisone. This along with a reported increase in the half life of radiolabelled cortisol suggests a reduction of 11 β-HSD type 2 activity [12].

### Rationale for the Development of 11 β-HSD Inhibitors

As said earlier cortisol opposes the action of insulin meaning a stimulation of hepatic gluconeogenesis, inhibition of peripheral glucose uptake and increased blood glucose concentration. The effects of cortisol appear to be enhanced in patients suffering from glucose intolerance or diabetes mellitus. Inhibition of the enzyme 11 β-HSD type 1 would increase glucose uptake and inhibit hepatic gluconeogenesis, giving a reduction in circulatory glucose levels. The development of a potent 11 β-HSD type 1 inhibitor could therefore have considerable therapeutic potential for conditions associated with elevated blood glucose levels.

An excess in glucocorticoids can result in neuronal dysfunctions and also impair cognitive functions. A specific 11 β-HSD type 1 inhibitor might be of some importance by reducing neuronal dysfunctions and the loss of cognitive functions associated with ageing, by blocking the conversion of cortisone to cortisol.

Glucocorticoids also have an important role in regulating part of the immune response [13]. Glucocorticoids can suppress the production of cytokines and regulate the receptor levels. They are also involved in determining whether T-helper (Th) lymphocytes progress into either Th1 or Th2 phenotype. These two different types of Th cells secrete a different profile of cytokines, Th2 is predominant in a glucocorticoid environment. By inhibiting 11 β-HSD type 1, Th1 cytokine response would be favoured. It is also possible to inhibit 11 β-HSD type 2, thus by inhibiting the inactivation of cortisol, it may be possible to potentiate the anti-inflammatory effects of glucocorticoids.

Aspects of the invention are defined in the appended claims.

### SUMMARY ASPECTS OF THE PRESENT INVENTION

In one aspect the present invention provides a compound of formula

R₁—CO—X—Y—Z—R₂

wherein

X and Z are each optional groups independently selected from saturated or unsaturated carbon chains having a length of 1 to 3 carbons

Y is SO, S, SO₂, CH═CH, CH₂CH₂ or O

R₁ is selected from the following groups

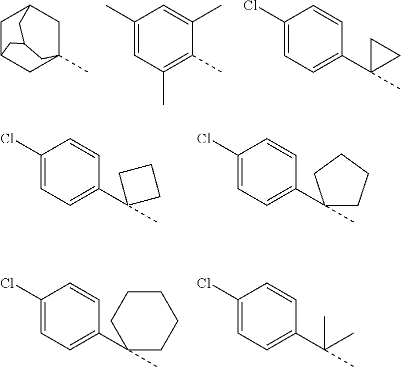

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

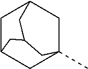

### and —CO—X—Y—Z— is CO—CH₂—SO, CO—CH₂—S, or CO—CH₂—SO₂,

R₂ is other than

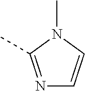

and
(ii) when R₁ is

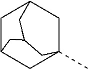

and —CO—X—Y—Z— is —CO—CH₂—O—, R₂ is other than

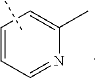

In one aspect the present invention provides a pharmaceutical composition comprising (a) a compound of formula

R₁—CO—X—Y—Z—R₂

wherein

X and Z are each optional groups independently selected from saturated or unsaturated carbon chains having a length of 1 to 3 carbons

Y is SO, S, SO₂, CH═CH, CH₂CH₂ or O

R₁ is selected from the following groups

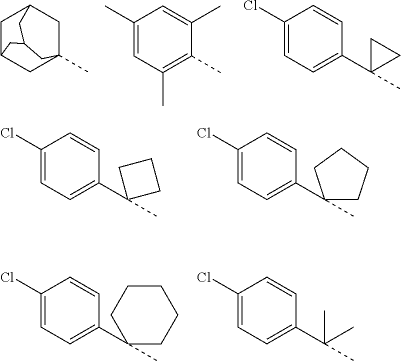

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

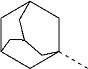

and —CO—X—Y—Z— is CO—CH₂—SO, CO—CH₂—S, or CO—CH₂—SO₂, R₂ is other than

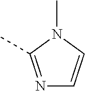

and
(ii) when R₁ is

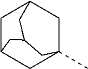

and —CO—X—Y—Z— is —CO—CH₂—O—, R₂ is other than

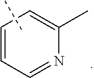

(b) optionally admixed with a pharmaceutically acceptable carrier, diluent, excipient or adjuvant.

In one aspect the present invention provides a compound for use in medicine wherein the compound is of formula

R₁—CO—X—Y—Z—R₂

wherein

X and Z are each optional groups independently selected from saturated or unsaturated carbon chains having a length of 1 to 3 carbons

Y is SO, S, SO₂, CH═CH, CH₂CH₂ or O

R₁ is selected from the following groups

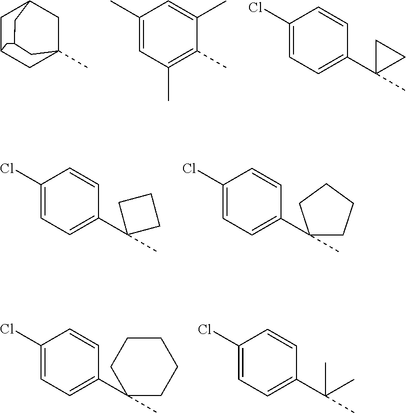

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

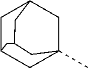

and —CO—X—Y—Z— is CO—CH₂—SO, CO—CH₂—S, or CO—CH₂—SO, R₂ is other than

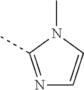

and
(ii) when R₁ is

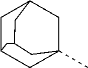

and —CO—X—Y—Z— is —CO—CH₂—O—, R₂ is other than

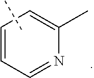

In one aspect the present invention provides a use of a compound in the manufacture of a medicament for use in the therapy of a condition or disease associated with 11β-HSD, wherein the compound is of formula

R₁—CO—X—Y—Z—R₂

wherein

X and Z are each optional groups independently selected from saturated or unsaturated carbon chains having a length of 1 to 3 carbons

Y is SO, S, SO₂, CH═CH, CH₂CH₂ or O

R₁ is selected from the following groups

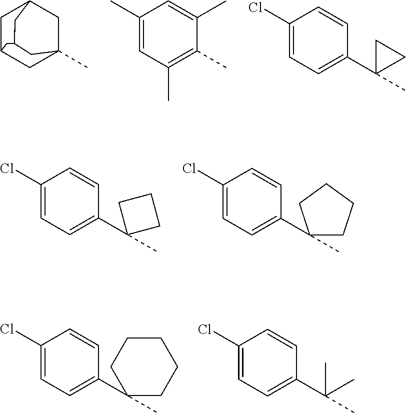

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

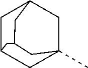

and —CO—X—Y—Z— is CO—CH₂—SO, CO—CH₂—S, or CO—CH₂—SO₂, R₂ is other than

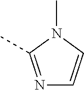

and
(ii) when R₁ is

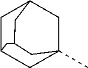

and —CO—X—Y—Z— is —CO—CH₂—O—, R₂ is other than

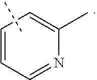

### SOME ADVANTAGES

One key advantage of the present invention is that the compounds of the present invention can act as 11β-HSD inhibitors. The compounds may inhibit the interconversion of inactive 11-keto steroids with their active hydroxy equivalents. Thus present invention provides methods by which the conversion of the inactive to the active form may be controlled, and useful therapeutic effects which may be obtained as a result of such control. More specifically, but not exclusively, the invention is concerned with interconversion between cortisone and cortisol in humans.

Another advantage of the compounds of the present invention is that they may be potent 11β-HSD inhibitors in vivo.

Some of the compounds of the present invention are also advantageous in that they may be orally active.

The present invention may provide for a medicament for one or more of (i) regulation of carbohydrate metabolism, (ii) regulation of protein metabolism, (iii) regulation of lipid metabolism, (iv) regulation of normal growth and/or development, (v) influence on cognitive function, (vi) resistance to stress and mineralocorticoid activity.

Some of the compounds of the present invention may also be useful for inhibiting hepatic gluconeogenesis. The present invention may also provide a medicament to relieve the effects of endogenous glucocorticoids in diabetes mellitus, obesity (including centripetal obesity), neuronal loss and/or the cognitive impairment of old age. Thus, in a further aspect, the invention provides the use of an inhibitor of 11β-HSD in the manufacture of a medicament for producing one or more therapeutic effects in a patient to whom the medicament is administered, said therapeutic effects selected from inhibition of hepatic gluconeogenesis, an increase in insulin sensitivity in adipose tissue and muscle, and the prevention of or reduction in neuronal loss/cognitive impairment due to glucocorticoid-potentiated neurotoxicity or neural dysfunction or damage.

From an alternative point of view, the invention provides a method of treatment of a human or animal patient suffering from a condition selected from the group consisting of: hepatic insulin resistance, adipose tissue insulin resistance, muscle insulin resistance, neuronal loss or dysfunction due to glucocorticoid potentiated neurotoxicity, and any combination of the aforementioned conditions, the method comprising the step of administering to said patient a medicament comprising a pharmaceutically active amount of a compound in accordance with the present invention.

Some of the compounds of the present invention may be useful for the treatment of cancer, such as breast cancer, as well as (or in the alternative) non-malignant conditions, such as the prevention of auto-immune diseases, particularly when pharmaceuticals may need to be administered from an early age.

### DETAILED ASPECTS OF THE PRESENT INVENTION

As previously mentioned, in one aspect the present invention provides a compound as defined above. The compound is a compound of formula

R₁—CO—X—Y—Z—R₂

wherein

X and Z are each optional groups independently selected from saturated or unsaturated carbon chains having a length of 1 to 3 carbons

Y is SO, S, SO₂, CH═CH, CH₂CH₂ or O

R₁ is selected from the following groups

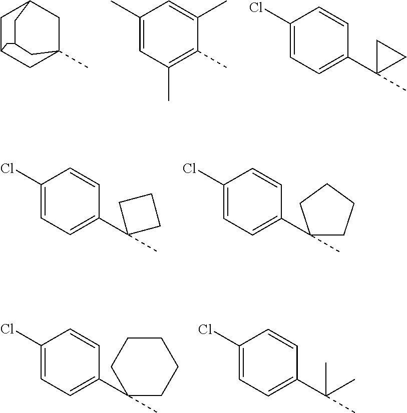

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

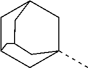

and —CO—X—Y—Z— is CO—CH₂—SO, CO—CH₂—S, or CO—CH₂—SO₂, R₂ is other than

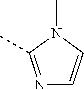

and
(ii) when R₁ is

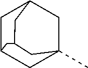

and —CO—X—Y—Z— is —CO—CH₂—O—, R₂ is other than

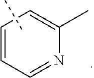

This compound and preferred compounds as defined herein are described as the ‘present compound’.

As previously mentioned, in one aspect the present invention provides a pharmaceutical composition comprising

(i) the present compound
(ii) optionally admixed with a pharmaceutically acceptable carrier, diluent, excipient or adjuvant.

As previously mentioned, in one aspect the present invention provides the present compound for use in medicine.

As previously mentioned, in one aspect the present invention provides a use of the present compound in the manufacture of a medicament for use in the therapy of a condition or disease associated with 11β-HSD.

In one aspect the present invention provides the present compound for use in the therapy of a condition or disease associated with 11β-HSD.

In one aspect the present invention provides a use of the present compound in the manufacture of a medicament for use in the therapy of a condition or disease associated with adverse 11β-HSD levels.

In one aspect the present invention provides the present compound for use in the therapy of a condition or disease associated with adverse 11β-HSD levels.

In one aspect the present invention provides a use of the present compound in the manufacture of a pharmaceutical for modulating 11β-HSD activity.

In one aspect the present invention provides the present compound for modulating 11β-HSD activity.

In one aspect the present invention provides a use of the present compound in the manufacture of a pharmaceutical for inhibiting 11β-HSD activity.

In one aspect the present invention provides the present compound for inhibiting 11β-HSD activity.

In one aspect the present invention provides a use of the present compound in the manufacture of a medicament for use in the therapy of a condition or disease selected from the group consisting of metabolic disorders such as diabetes and obesity; cardiovascular disorders such as hypertension; glaucoma; inflammatory disorders such as arthritis or asthma; immune disorders; bone disorders such as osteoporosis; cancer; intra-uterine growth retardation; apparent mineralocorticoid excess syndrome (AME); polycystic ovary syndrome (PCOS); hirsutism; acne; oligo- or amenorrhea; adrenal cortical adenoma and carcinoma; Cushing's syndrome; pituitary tumours; invasive carcinomas; breast cancer; and endometrial cancer.

In one aspect the present invention provides the present compound for use in the therapy of a condition or disease selected from the group consisting of metabolic disorders such as diabetes and obesity; cardiovascular disorders such as hypertension; glaucoma; inflammatory disorders such as arthritis or asthma; immune disorders; bone disorders such as osteoporosis; cancer; intra-uterine growth retardation; apparent mineralocorticoid excess syndrome (AME); polycystic ovary syndrome (PCOS); hirsutism; acne; oligo- or amenorrhea; adrenal cortical adenoma and carcinoma; Cushing's syndrome; pituitary tumours; invasive carcinomas; breast cancer; and endometrial cancer.

For ease of reference, these and further aspects of the present invention are now discussed under appropriate section headings. However, the teachings under each section are not necessarily limited to each particular section.

### Preferable Aspects

### Compound

As previously mentioned, in one aspect the present invention provides a compound of formula

R₁—CO—X—Y—Z—R₂

wherein

X and Z are each optional groups independently selected from saturated or unsaturated carbon chains having a length of 1 to 3 carbons

Y is SO, S, SO₂, CH═CH, CH₂CH₂ or O

R₁ is selected from the following groups

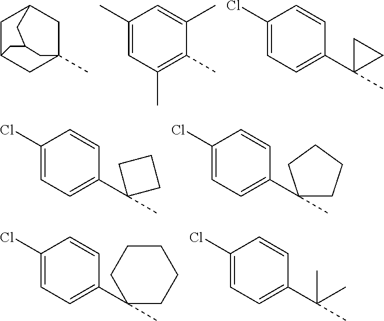

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

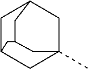

and —CO—X—Y—Z— is CO—CH₂—SO, CO—CH₂—S, or CO—CH₂—SO₂, R₂ is other than

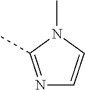

and
(ii) when R₁ is

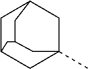

and —CO—X—Y—Z— is —CO—CH₂—O—, R₂ is other than

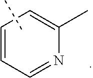

In one aspect the compound of the present invention is of formula

R₁—CO—X—Y—Z—R₂

wherein X and Z are independently selected from saturated or unsaturated carbon chains having a length of 1 to 3 carbons, and

Y is SO, S, SO₂, CH═CH, CH₂CH₂ or O.

In one aspect the compound of the present invention is of formula

R₁—CO—X—Y—R₂

wherein X is selected from saturated or unsaturated carbon chains having a length of 1 to 3 carbons, and

Y is SO, S, SO₂, CH═CH, CH₂CH₂ or O.

In one aspect the compound of the present invention is of formula

R₁—CO—Y—Z—R₂

wherein Z is selected from saturated or unsaturated carbon chains having a length of 1 to 3 carbons, and

Y is SO, S, SO₂, CH═CH, CH₂CH₂ or O.

In one aspect the compound of the present invention is of formula

R₁—CO—Y—R₂

wherein Y is SO, S, SO₂, CH═CH, CH₂CH₂ or O.

### Group R₁

R₁ is a group selected from the following

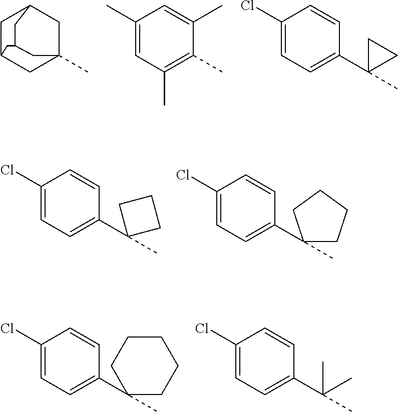

wherein [symbol] denotes the point of attachment.

[symbol]

Thus the present invention provides a compound of the formula

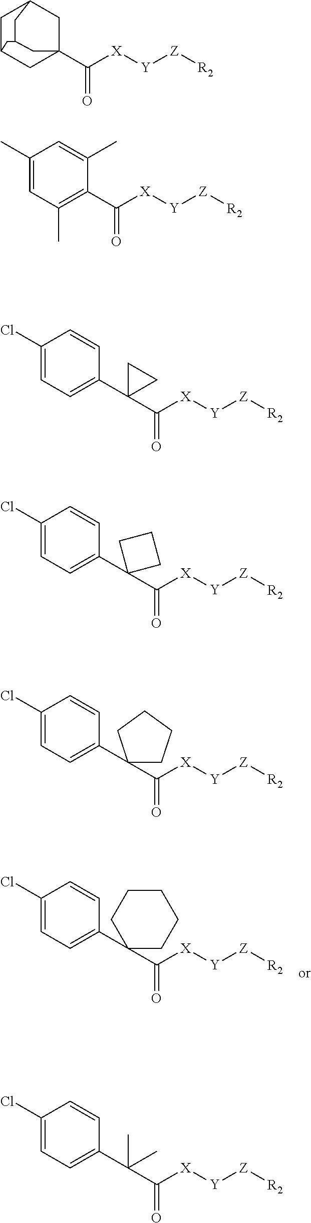

X and Z are each optional groups independently selected from saturated or unsaturated carbon chains having a length of 1 to 3 carbons

Y is SO, S, SO₂, CH═CH, CH₂CH₂ or O.

In one aspect R₁ is

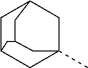

such that the present invention provides a compound of the formula

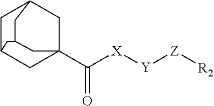

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is

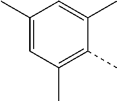

such that the present invention provides a compound of the formula

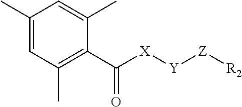

wherein X and Z are each optional groups independently selected from saturated or unsaturated carbon chains having a length of 1 to 3 carbons

Y is SO, S, SO₂, CH═CH, CH₂CH₂ or O.

In one aspect, R₁ is a group selected from the following

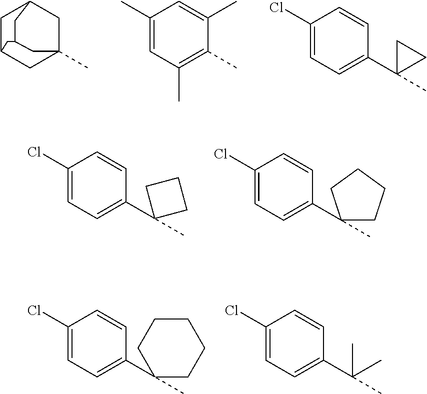

wherein [symbol] denotes the point of attachment.

[symbol]

In one aspect R₁ is selected from the following groups

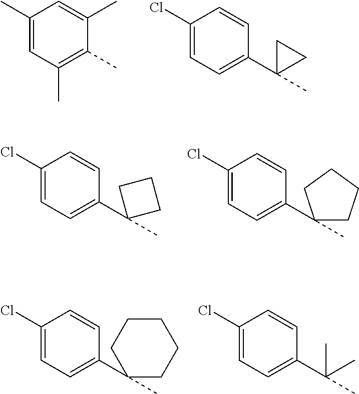

such that the present invention provides a compound of the formula

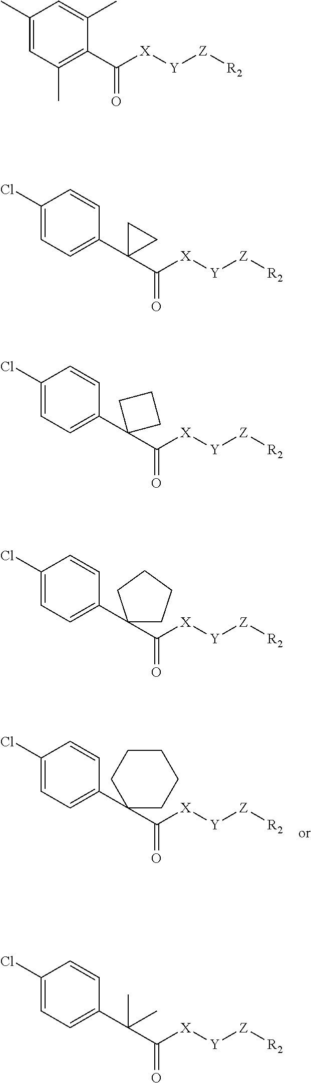

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is selected from the following groups

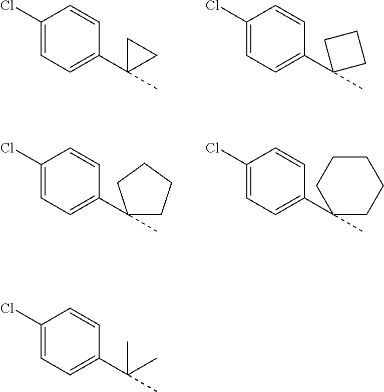

such that the present invention provides a compound of the formula

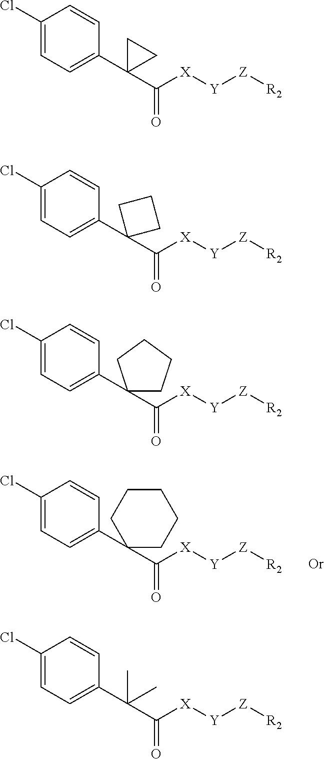

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is

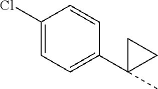

such that the present invention provides a compound of the formula

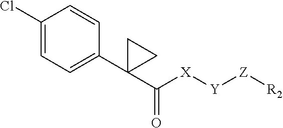

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is

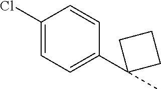

such that the present invention provides a compound of the formula

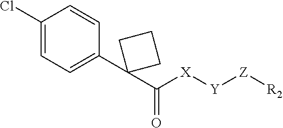

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is

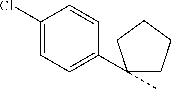

such that the present invention provides a compound of the formula

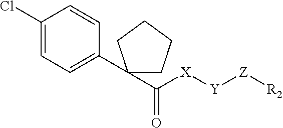

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is

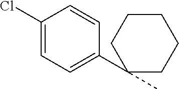

such that the present invention provides a compound of the formula

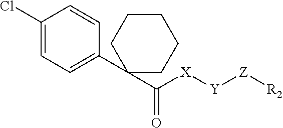

wherein X and Z are each optional groups independently selected from saturated or unsaturated carbon chains having a length of 1 to 3 carbons

Y is SO, S, SO₂, CH═CH, CH₂CH₂ or O.

In one aspect R₁ is

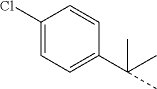

such that the present invention provides a compound of the formula

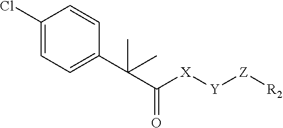

wherein X and Z are each optional groups independently selected from saturated or unsaturated carbon chains having a length of 1 to 3 carbons

Y is SO, S, SO₂, CH═CH, CH₂CH₂ or O.

### Group X

X is an optional group selected from saturated or unsaturated carbon chains having a length of 1 to 3 carbons.

In one aspect X is a group selected from saturated or unsaturated carbon chains having a length of 1 to 3 carbons. In this aspect, group X is not optional.

In a further aspect group X is not present. In this aspect group X represents a bond. Thus in one aspect, X and Z are each groups independently selected from a bond and saturated or unsaturated carbon chains having a length of 1 to 3 carbons.

In a preferred aspect X is selected from or when present is selected from C₁₋₃ alkylene.

In a further preferred aspect X is selected from C₁₋₃ alkylene.

In a preferred aspect X is selected from or when present is selected from CH₂ and C(CH₃)₂.

In a preferred aspect X is selected from CH₂ and C(CH₃)₂.

In one preferred aspect X is CH₂.

### Group Z

Z is an optional group selected from saturated or unsaturated carbon chains having a length of 1 to 3 carbons.

In one aspect Z is a group selected from saturated or unsaturated carbon chains having a length of 1 to 3 carbons. In this aspect, group Z is not optional.

In a further aspect group Z is not present. In this aspect group Z may represent a bond. Thus in one aspect, X and Z are each optional groups independently selected from a bond and saturated or unsaturated carbon chains having a length of 1 to 3 carbons.

In a preferred aspect Z is selected from or when present is selected from C₁₋₃ alkylene.

In a further preferred aspect Z is selected from C₁₋₃ alkylene.

In a preferred aspect Z is selected from or when present is CH₂.

In one preferred aspect Z is CH₂.

### Group Y

Group Y is selected from SO, S, SO₂, CH═CH, CH₂CH₂ or O.

In one aspect group Y is SO.

In one aspect group Y is S.

In one aspect group Y is SO₂.

In one aspect group Y is CH═CH.

In one aspect group Y is CH₂CH₂.

In one aspect group Y is O.

In one aspect group when Y is CH═CH or CH₂CH₂, X and Z are not present i.e. are bonds.

In a further aspect the present invention provides a compound of formula

R₁—CO—X—Y—Z—R₂

wherein
(A) X and Z are each optional groups independently selected from saturated or unsaturated carbon chains having a length of 1 to 3 carbons, and
   Y is SO, S, SO₂, or O, or
(B) X and Z are each groups independently selected from saturated or unsaturated carbon chains having a length of 1 to 3 carbons, and
   Y is CH═CH, or CH₂CH₂

R₁ is selected from the following groups

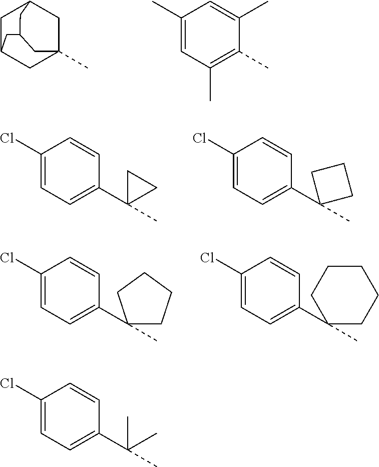

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

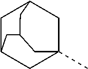

and —CO—X—Y—Z— is CO—CH₂—SO, CO—CH₂—S, or CO—CH₂—SO₂, R₂ is other than

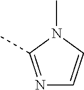

and
(ii) when R₁ is

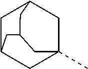

and —CO—X—Y—Z— is —CO—CH₂—O—, R₂ is other than

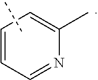

### Group R₂

R₂ is a heteroaryl group comprising an optionally substituted 5 or 6 membered ring, which ring contains only carbon and at least one nitrogen, or contains only carbon, and at least two nitrogens and at least one sulphur.

In one preferred aspect R₂ is a heteroaryl group comprising an optionally substituted 5 or 6 membered ring, which ring which ring contains only carbon and at least one nitrogen.

In one preferred aspect R₂ is a heteroaryl group comprising an optionally substituted 5 or 6 membered ring, which ring or contains only carbon, and at least two nitrogens and at least one sulphur.

Preferably R₂ is a heteroaryl group comprising an optionally substituted 5 membered ring which ring contains only carbon and at least one nitrogen.

Preferably R₂ is a heteroaryl group comprising an optionally substituted 5 membered ring which ring or contains only carbon, and at least two nitrogens and at least one sulphur.

Preferably R₂ is a heteroaryl group comprising an optionally substituted 6 membered ring which ring contains only carbon and at least one nitrogen.

In a preferred aspect R₂ is selected from
a heteroaryl group comprising an optionally substituted 5 membered ring which ring contains only carbon and at least one nitrogen
a heteroaryl group comprising an optionally substituted 5 membered ring which ring or contains only carbon, and at least two nitrogens and at least one sulphur and
a heteroaryl group comprising an optionally substituted 6 membered ring which ring contains only carbon and at least one nitrogen.

In the present specification, by the term heteroaryl group, it is meant an aryl ring containing as ring members at least carbon and one or more of N, S and O. This definition of heteroaryl group is applicable to all usage of the same (not only in respect of the R₂ group) and is subject to the other limitations thereon, such as those above in respect of specific R₂ groups containing only carbon and at least one nitrogen.

R₂ may be substituted or unsubstituted. Preferably R₂ is substituted.

A preferred R₂ group is an optionally substituted 5 or 6 membered heteroaryl ring selected from

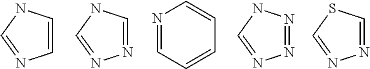

A preferred R₂ group is an optionally substituted 5 or 6 membered heteroaryl ring selected from

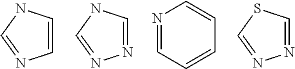

A preferred R₂ group is an optionally substituted 5 or 6 membered heteroaryl ring selected from

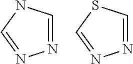

A preferred R₂ group is an optionally substituted 5 or 6 membered heteroaryl ring selected from

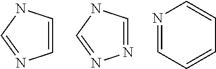

A preferred R₂ group is an optionally substituted

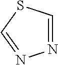

group.

A preferred R₂ group is an optionally substituted 5 or 6 membered heteroaryl ring selected from

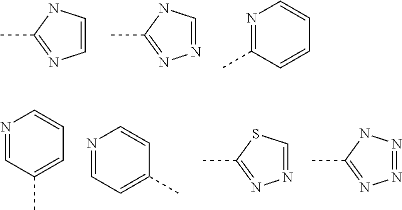

wherein [symbol] denotes the point of attachment.

A preferred R₂ group is an optionally substituted 5 or 6 membered heteroaryl ring selected from

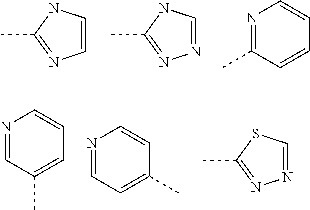

wherein [symbol] denotes the point of attachment.

A preferred R₂ group is an optionally substituted 5 or 6 membered heteroaryl ring selected from

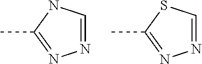

wherein [symbol] denotes the point of attachment.

A preferred R₂ group is an optionally substituted 5 or 6 membered heteroaryl ring selected from

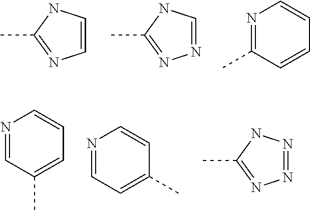

wherein [symbol] denotes the point of attachment.

A preferred R₂ group is an optionally substituted 5 or 6 membered heteroaryl ring selected from

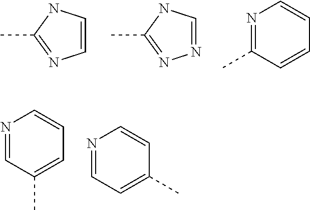

wherein [symbol] denotes the point of attachment.

A preferred R₂ group is an optionally substituted

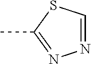

group, wherein [symbol] denotes the point of attachment.

The optional substitutents of the R₂ group are preferably independently selected from hydrocarbyl groups, halogens, hydroxyl, carbonyl, amines, and amides.

The optionally substituents of R₂ may together form a further ring fused to the 5 or 6 membered heteroaryl ring. Preferably the further fused ring is (itself) 5 or 6 membered ring. Preferably the further fused ring is (itself) an aryl ring. Preferably the ring members of the further fused ring are at least carbon and optionally one or hetero atoms selected from N, S and O. Preferably the further fused ring is a carbocyclic ring. Preferably the further fused ring is a 5 or 6 membered carbocyclic ring. Preferably the further fused ring is a 5 or 6 membered aryl ring. Preferably the further fused ring is a 5 or 6 membered carbocyclic aryl ring. Preferably the further fused ring is a phenyl group.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In some aspects of the present invention, one or more hydrocarbyl groups is independently selected from optionally substituted alkyl group, optionally substituted haloalkyl group, aryl group, alkylaryl group, alkylarylakyl group, and an alkene group.

In some aspects of the present invention, one or more hydrocarbyl groups is independently selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

In some aspects of the present invention, one or more hydrocarbyl groups is independently selected from alkene groups. Typical alkene groups include C₁-C₁₀ alkene group, C₁-C₆ alkene group, C₁-C₃ alkene group, such as C₁, C₂, C₃, C₄, C₅, C₆, or C₇ alkene group. In a preferred aspect the alkene group contains 1, 2 or 3 C═C bonds.

In a preferred aspect the alkene group contains 1 C═C bond. In some preferred aspects at least one C═C bond or the only C═C bond is to the terminal C of the alkene chain, that is the bond is at the distal end of the chain to the ring system.

In some aspects of the present invention, one or more hydrocarbyl groups is independently selected from oxyhydrocarbyl groups.

One particular hydrocarbyl group is an oxyhydrocarbyl group. The term “oxyhydrocarbyl” group as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Typically, the oxyhydrocarbyl group is of the formula C₁₋₆O (such as a C₁₋₃O).

In a preferred aspect the or each optional substituent of the R₂ group is independently selected from oxy groups, ether groups, thioether groups, aryl groups, aryl groups substituted with one or alkyl groups (preferably C₁₋₅ alkyl groups) or halogens, alkyl groups, alkoxy groups, halo alkyl groups, halogens, amides and carbonyl groups or together form an aryl group fused to the 5 or 6 membered heteroaryl ring.

In a preferred aspect the or each optional substituent of the R₂ group is independently selected from oxy groups, alkyl groups, alkoxy groups, halo alkyl groups, halogens, amides and carbonyl groups or together form an aryl group fused to the 5 or 6 membered heteroaryl ring.

In a preferred aspect the or each optional substituent of the R₂ group is independently selected from C₁₋₅ alkyl groups, C₃₋₆ cycloalkyl groups, ether groups containing from 1 to 5 carbons, thioether groups containing from 1 to 5 carbons, C₁₋₅ alkoxy groups, C₁₋₅ haloalkyl group, halogens, oxy group, amines, phenyl, furan, thiophene, —(C₁₋₅ alkyl)-phenyl groups substituted by one or more halogens [—(C₁₋₅ alkyl)-phenyl denotes a C₁₋₅ alkyl radical attached to optional group Z and to a phenyl group], amides, alkyl amides, dialkyl amides, acylamides or together form a phenyl group fused to the 5 or 6 membered heteroaryl ring.

In a preferred aspect the or each optional substituent of the R₂ group is independently selected from C₁₋₅ alkyl groups, C₁₋₅ alkoxy groups, C₁₋₅ haloalkyl group, halogens, oxy group, amides, alkyl amides and dialkyl amides or together form a phenyl group fused to the 5 or 6 membered heteroaryl ring.

In a preferred aspect the or each optional substituent of the R₂ group is independently selected from methyl, methoxy, oxy, chloro, CH(CH₃)₂, —S-Me, —CH₂—O-Me, CF₃, NMe₂, COOH, C═ONH₂, C═ONHMe, C═ONMe₂, C═ONHCH₂CH₃, —NH₂, phenyl, furan, thiophene, —NH—C═OMe, —NH—C═O-cyclopropane, cyclopropane, CH₂-4-chlorophenyl, or together form a phenyl group fused to the 5 or 6 membered heteroaryl ring.

In a preferred aspect the or each optional substituent of the R₂ group is independently selected from methyl, methoxy, oxy, chloro, CF₃, COOH, C═ONH₂, C═ONHMe, C═ONMe₂, and C═ONHCH₂CH₃ or together form a phenyl group fused to the 5 or 6 membered heteroaryl ring.

In a highly preferred aspect the R₂ group is selected from

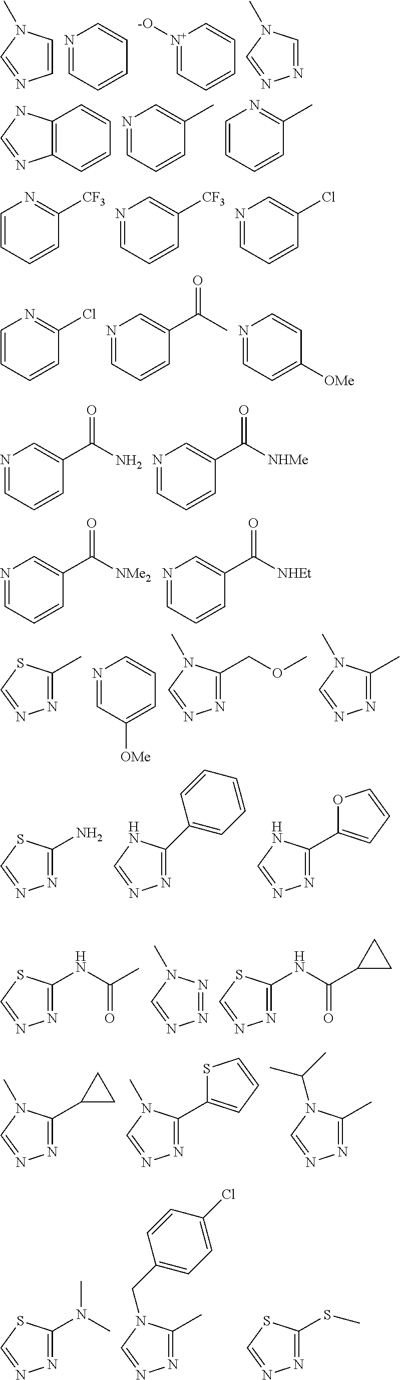

In a highly preferred aspect the R₂ group is selected from

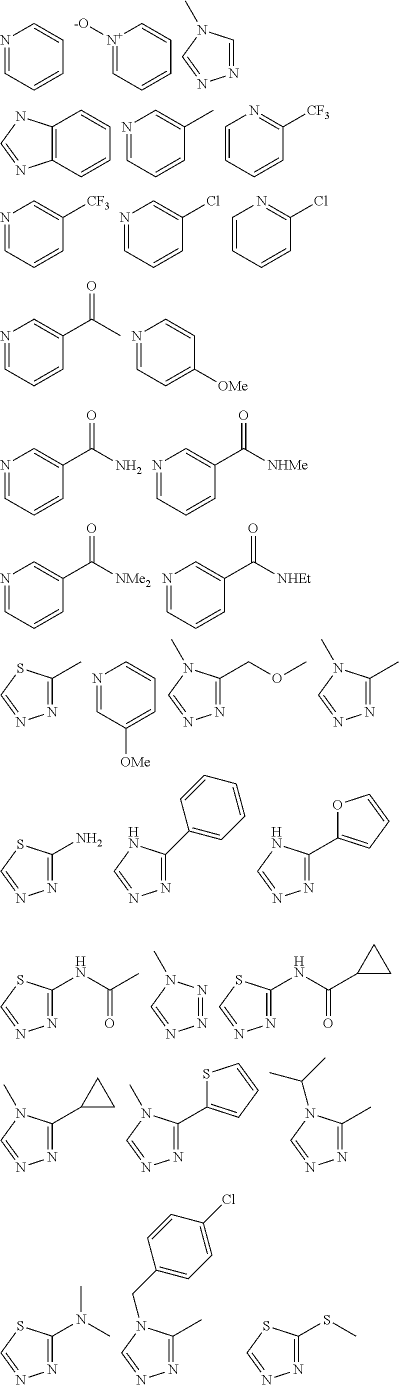

In a highly preferred aspect the R₂ group is selected from

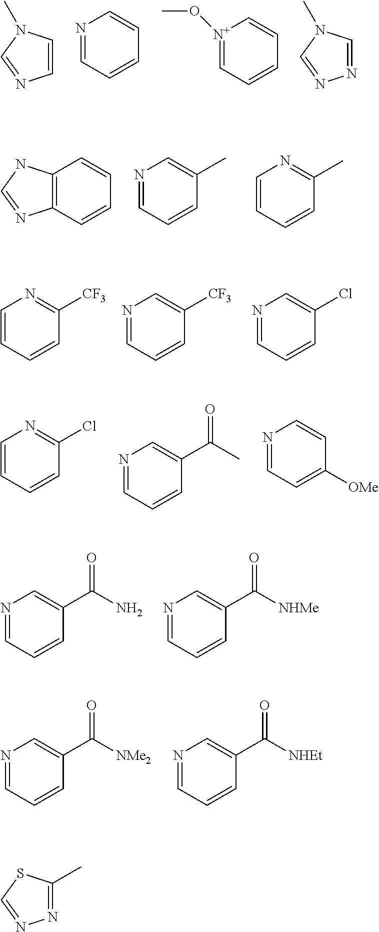

In a highly preferred aspect the R₂ group is selected from

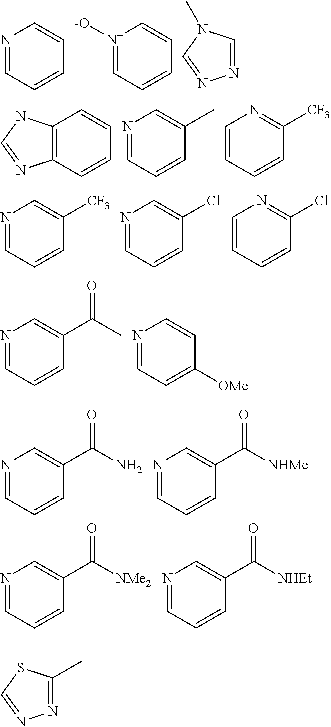

In a highly preferred aspect the R₂ group is selected from

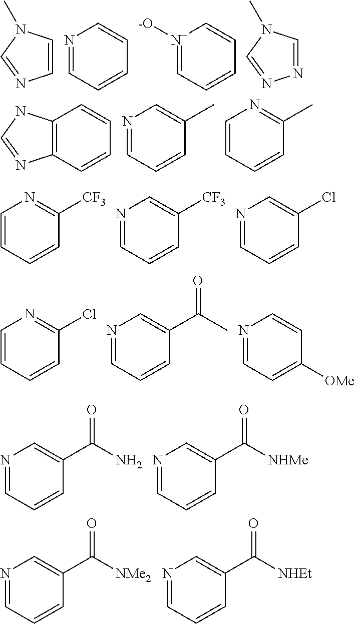

In a further highly preferred aspect the R₂ group is

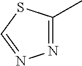

In a further highly preferred aspect the R₂ group is selected from

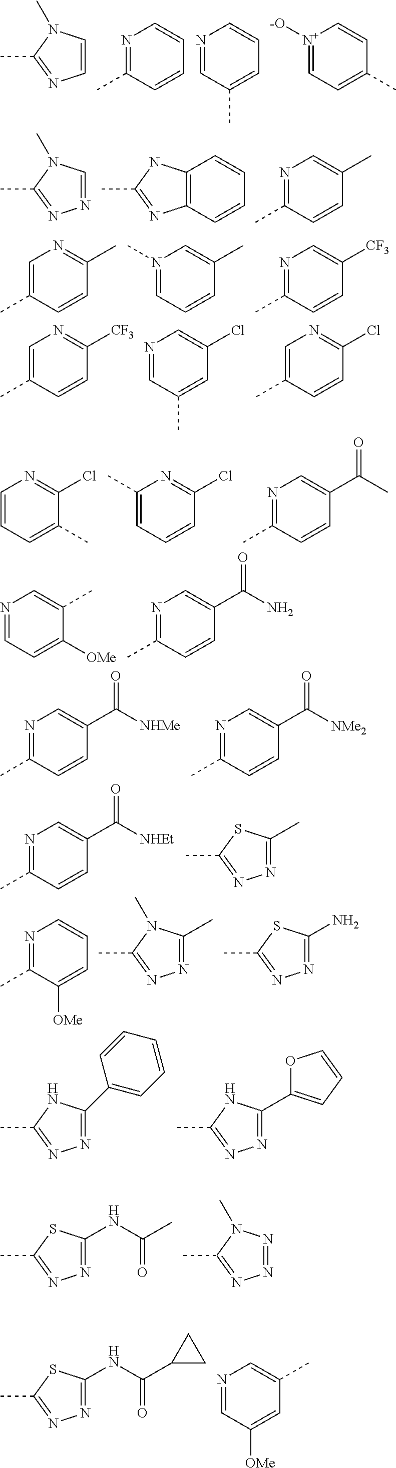

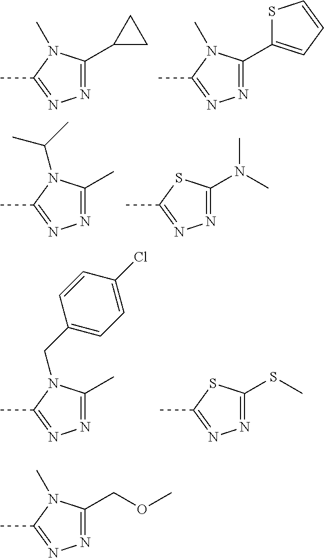

wherein [symbol] denotes the point of attachment.

In a further highly preferred aspect the R₂ group is selected from

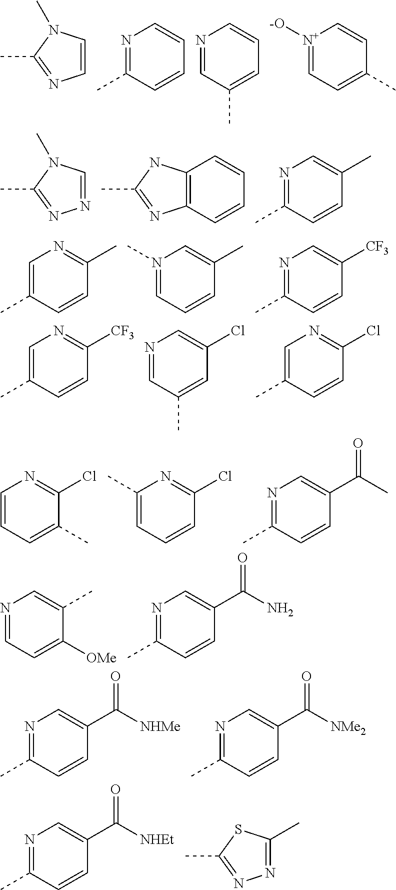

wherein [symbol] denotes the point of attachment.

In a further highly preferred aspect the R₂ group is selected from

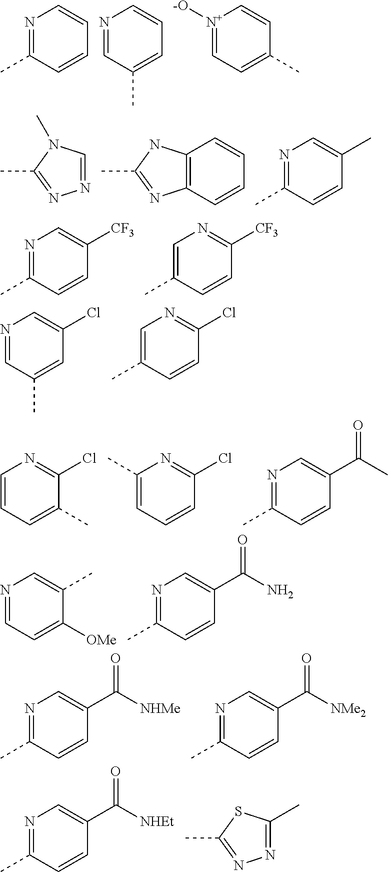

wherein [symbol] denotes the point of attachment.

In a further highly preferred aspect the R₂ group is selected from

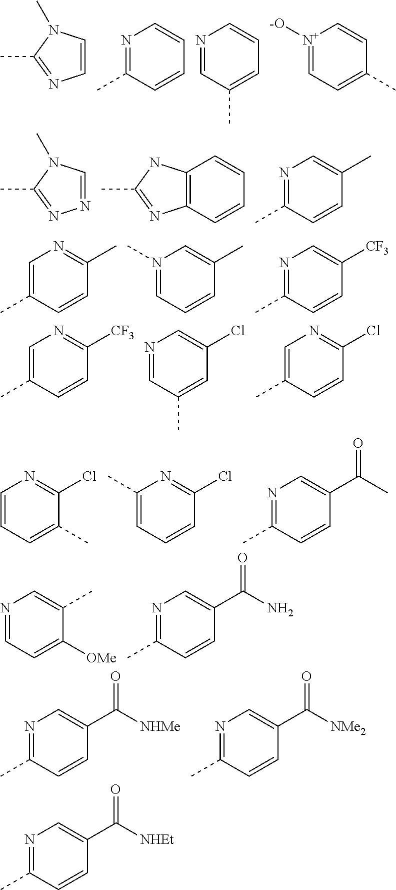

wherein [symbol] denotes the point of attachment.

In a further highly preferred aspect the R₂ group is selected from

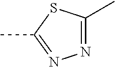

wherein [symbol] denotes the point of attachment.

### Further Aspects

For some applications, preferably the compounds have a reversible action.

For some applications, preferably the compounds have an irreversible action.

In one embodiment, the compounds of the present invention are useful for the treatment of breast cancer.

The compounds of the present invention may be in the form of a salt.

The present invention also covers novel intermediates that are useful to prepare the compounds of the present invention. For example, the present invention covers novel alcohol precursors for the compounds. The present invention also encompasses a process comprising precursors for the synthesis of the compounds of the present invention.

The compound of the present invention may have substituents other than those of the ring systems show herein. Furthermore the ring systems herein are given as general formulae and should be interpreted as such. The absence of any specifically shown substituents on a given ring member indicates that the ring member may substituted with any moiety of which H is only one example. Each ring system may contain one or more degrees of unsaturation, for example is some aspects one or more rings of a ring system is aromatic. Each ring system may be carbocyclic or may contain one or more hetero atoms.

The compound of the invention, in particular the ring systems of the compound of the invention may contain substituents other than those show herein. By way of example, these other substituents may be one or more of: one or more halo groups, one or more O groups, one or more hydroxy groups, one or more amino groups, one or more sulphur containing group(s), one or more hydrocarbyl group(s)—such as an oxyhydrocarbyl group.

In general terms the ring systems of the present compounds may contain a variety of non-interfering substituents. In particular, the ring systems may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

In a highly preferred aspect, the compound is of formula

R₁—CO—X—Y—Z—R₂

wherein

X is selected from C₁₋₃ alkylene;

Z is an optional group selected from C₁₋₃ alkylene; and

Y is SO, S, or SO₂.

More preferably X is selected from CH₂ and C(CH₃)₂ and Z is an optional CH₂ group.

In a highly preferred aspect, the compound is of formula

R₁—CO—X—O—Z—R₂

wherein

X is selected from C₁₋₃ alkylene;

Z is an optional group selected from C₁₋₃ alkylene.

More preferably wherein X is CH₂ and Z is an optional CH₂ group.

In a highly preferred aspect, the compound is of formula

R₁—CO—Y—R₂

wherein

Y is CH═CH or CH₂CH₂.

In a highly preferred aspect, —CO—X—Y—Z— is selected from COCH₂S, COCH₂SO, COCH₂SO₂, COCH₂SCH₂, COCH₂SOCH₂, COCH₂SO₂CH₂, COC(CH₃)₂SO, COCH₂O, COCH₂OCH₂, COCH═CH and COCH₂CH₂.

Thus in a preferred aspect the present invention provides a compound selected from R₁—COCH₂S—R₂, R₁—COCH₂SO—R₂, R₁—COCH₂SO₂—R₂, R₁—COCH₂SCH₂—R₂, R₁—COCH₂SOCH₂—R₂, R₁—COCH₂SO₂CH₂—R₂, R₁—COC(CH₃)₂SO—R₂, R₁—COCH₂O—R₂, R₁—COCH₂OCH₂—R₂, R₁—COCH═CH—R₂ and R₁—COCH₂CH₂—R₂.

wherein

R₁ is selected from the following groups

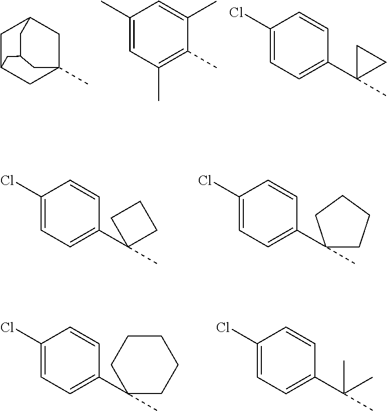

wherein [symbol] denotes the point of attachment

R₂ is a heteroaryl group comprising an optionally substituted 5 or 6 membered ring, which ring contains only carbon and at least one nitrogen, or contains only carbon, and at least two nitrogens and at least one sulphur; and

wherein
(i) when R₁ is

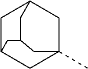

and —CO—X—Y—Z— is CO—CH₂—SO, CO—CH₂—S, or CO—CH₂—SO₂, R₂ is other than

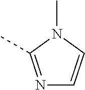

and
(ii) when R₁ is

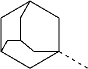

and —CO—X—Y—Z— is —CO—CH₂—O—, R₂ is other than

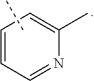

In a highly preferred aspect, the compounds of the present invention or for use in the present invention are selected from compounds of the formulae:

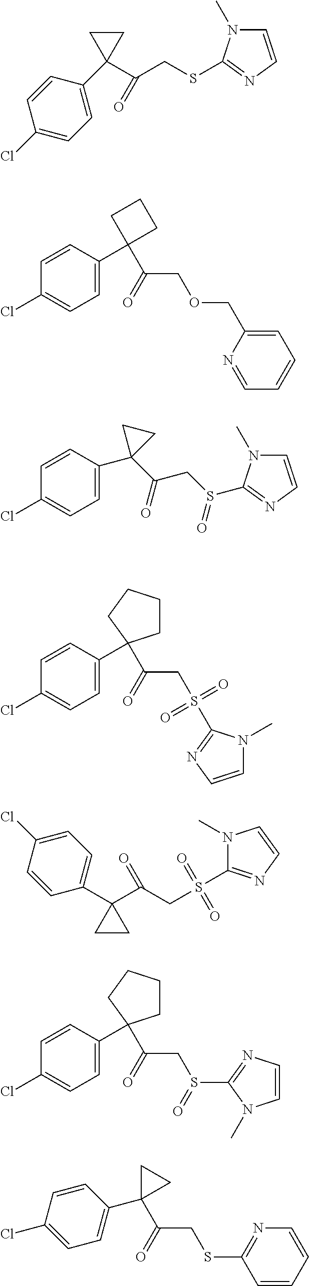

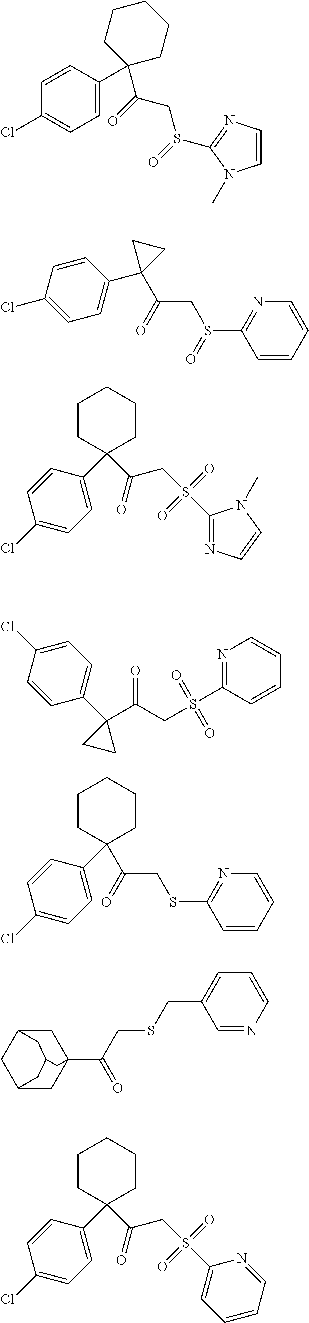

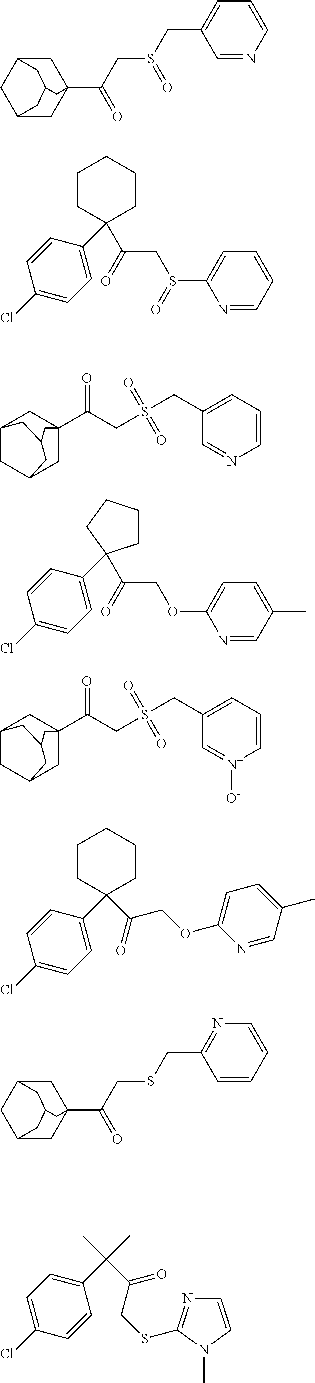

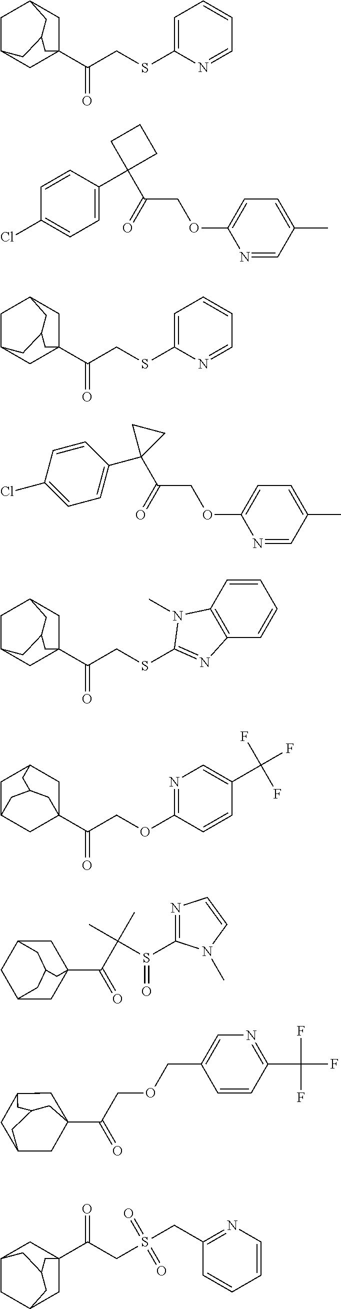

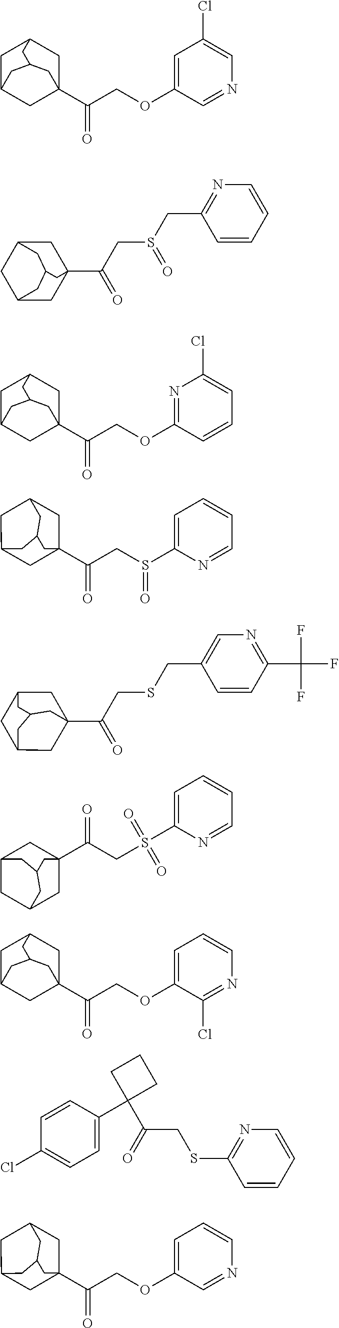

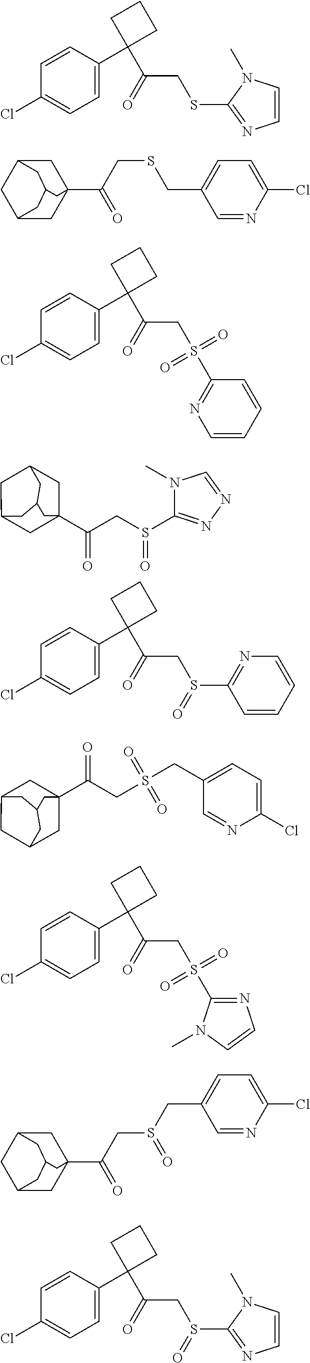

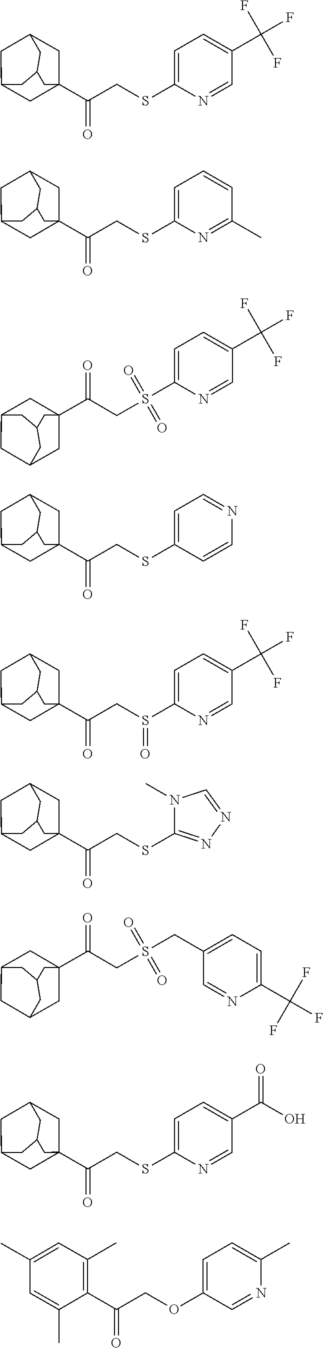

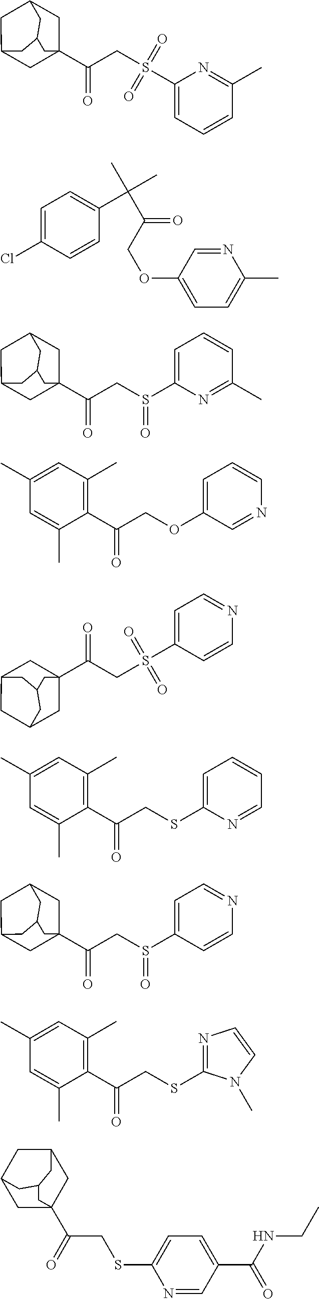

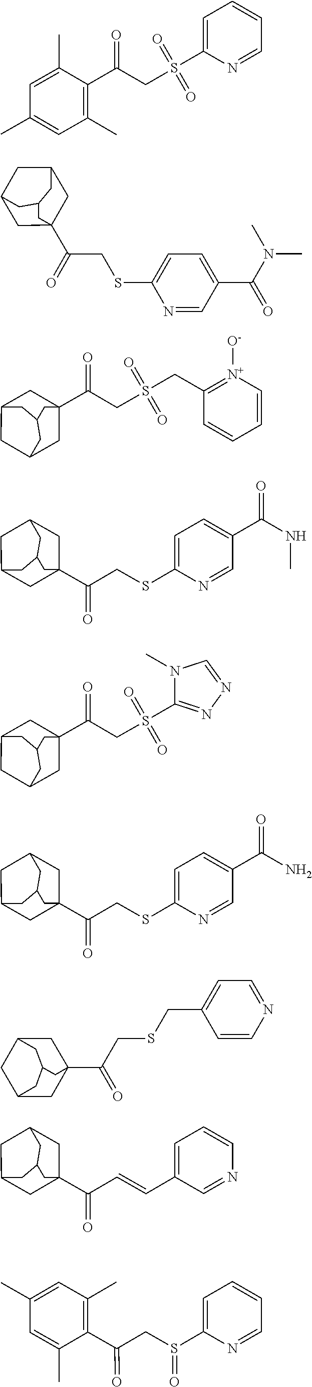

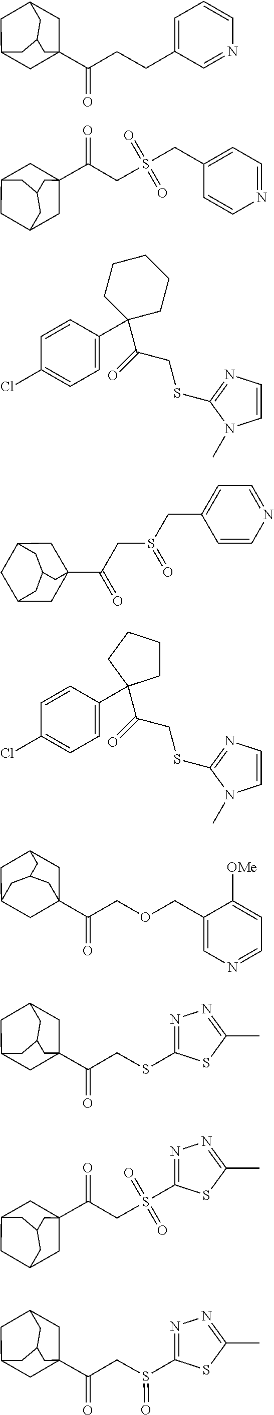

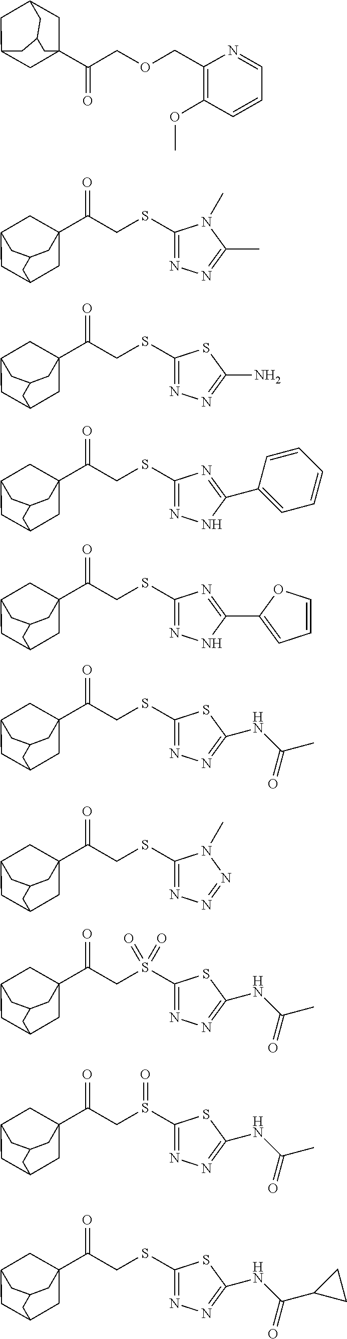

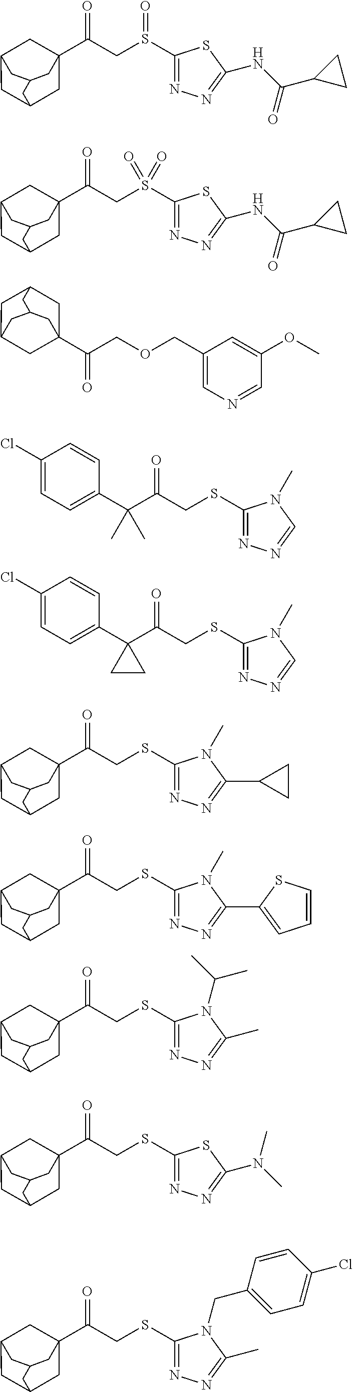

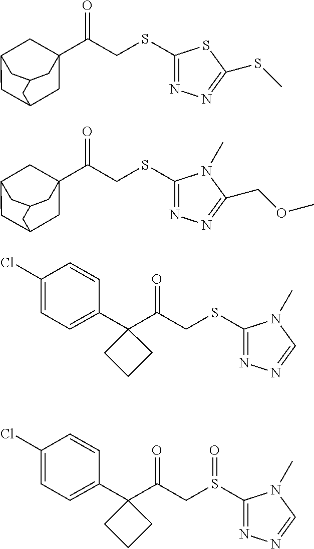

In a highly preferred aspect, the compounds of the present invention or for use in the present invention are selected from compounds of the formulae:

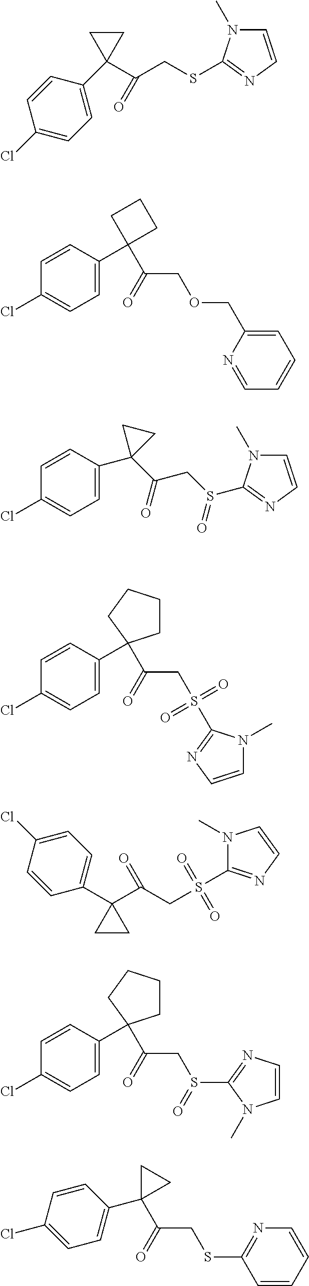

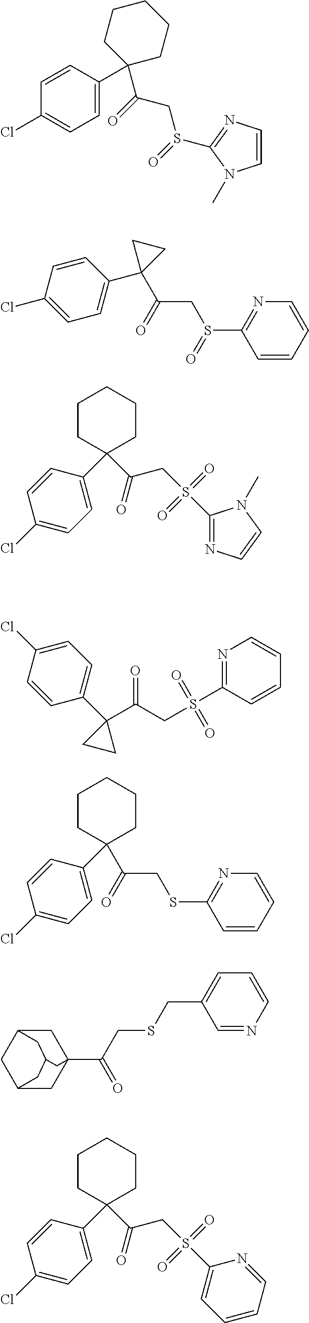

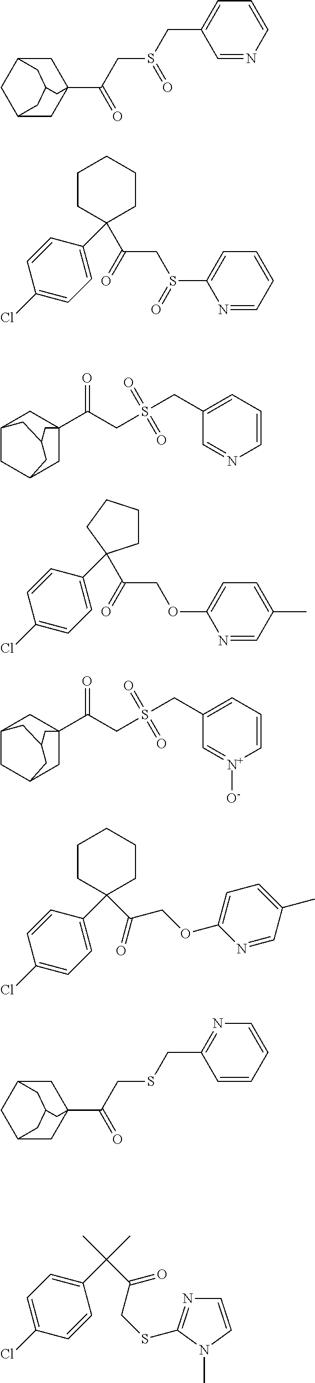

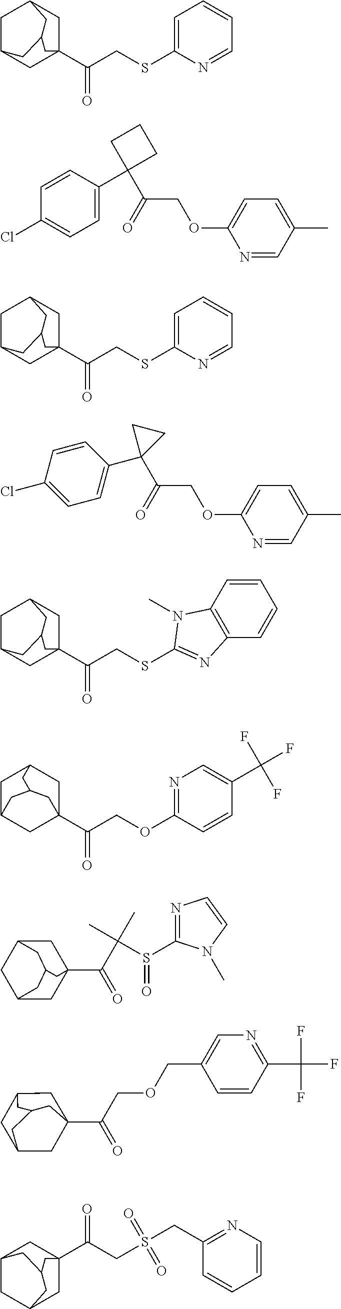

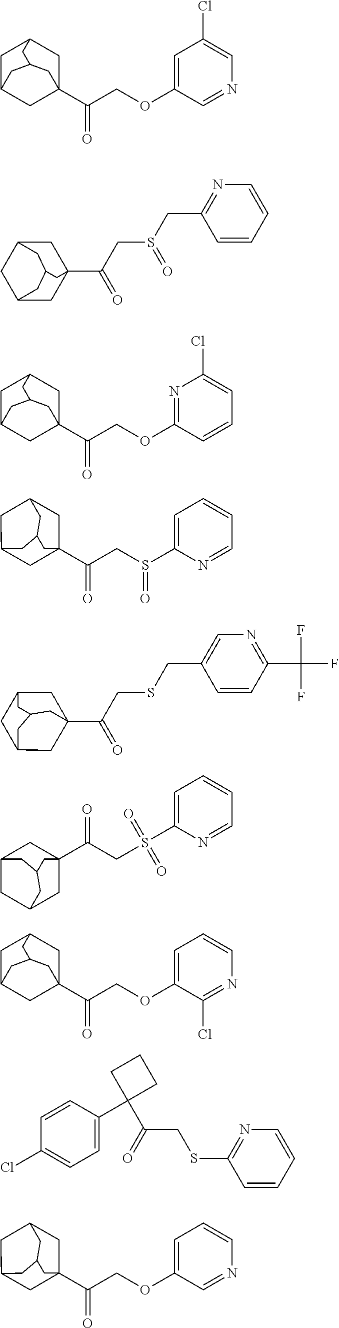

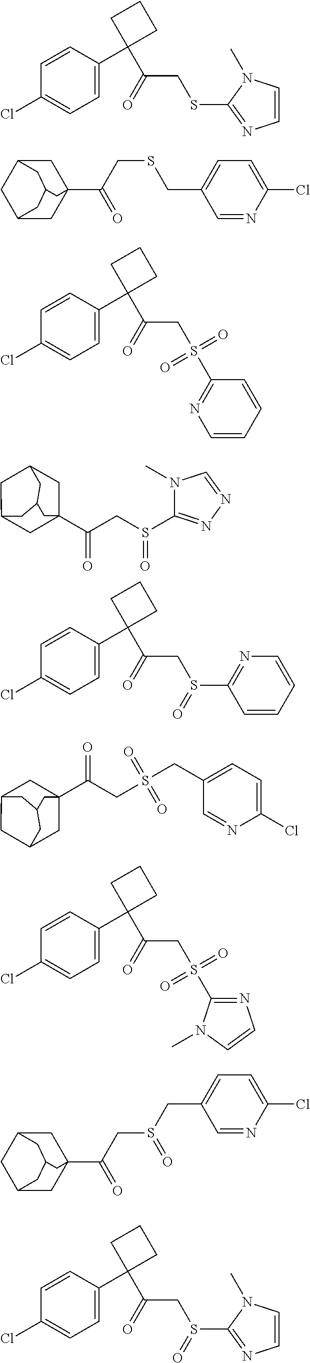

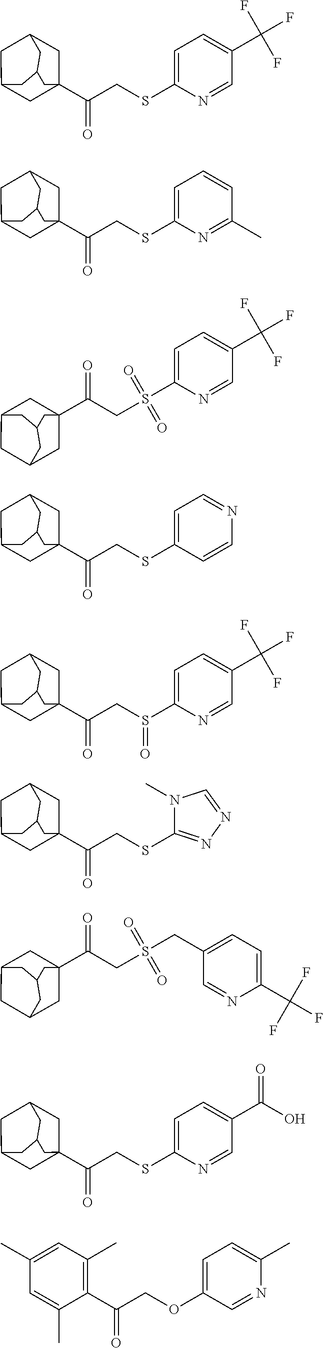

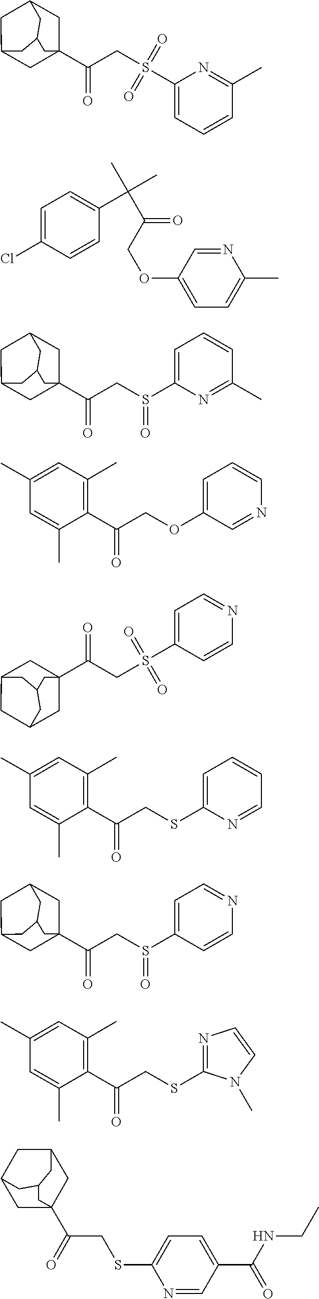

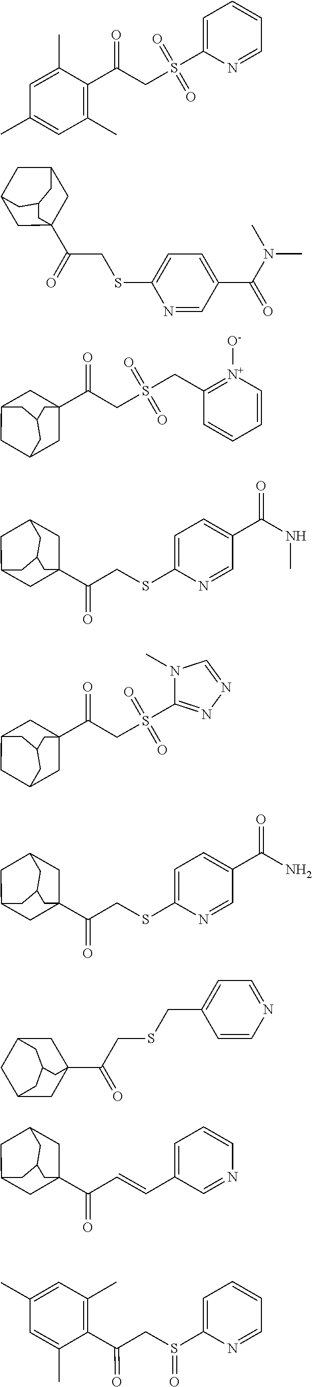

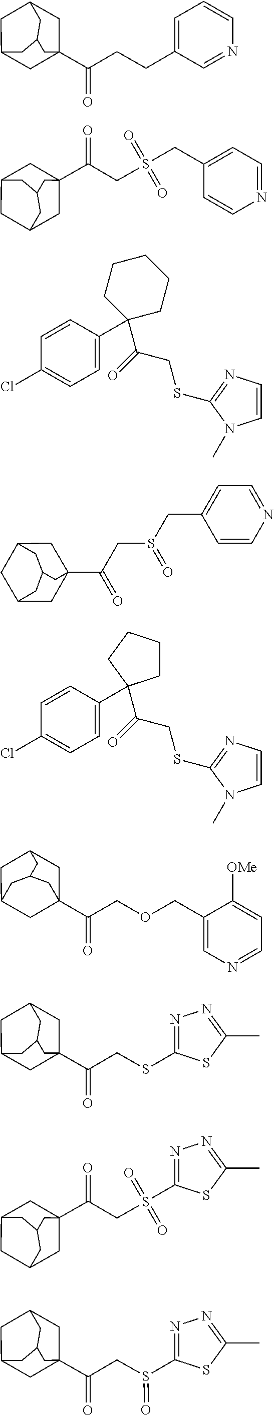

### Hydroxysteroid Dehydrogenase

11β Hydroxysteroid dehydrogenase may be referred to as “11β-HSD” or “HSD” for short.

In some aspects of the invention 11β-HSD is preferably 11β-HSD Type 1 (EC1.1.1.146).

In some aspects of the invention 11 β-HSD is preferably 11β-HSD Type 2 (EC1.1.1.146).

### Hydroxysteroid Dehydrogenase Inhibition

It is believed that some disease conditions associated with HSD activity are due to conversion of a inactive, cortisone to an active, cortisol. In disease conditions associated with HSD activity, it would be desirable to inhibit HSD activity.

Here, the term “inhibit” includes reduce and/or eliminate and/or mask and/or prevent the detrimental action of HSD.

### HSD Inhibitor

In accordance with the present invention, the compound of the present invention is capable of acting as an HSD inhibitor.

Here, the term “inhibitor” as used herein with respect to the compound of the present invention means a compound that can inhibit HSD activity—such as reduce and/or eliminate and/or mask and/or prevent the detrimental action of HSD. The HSD inhibitor may act as an antagonist.

The ability of compounds to inhibit hydroxysteroid dehydrogenase activity can be assessed using the suitable biological assay presented in the Examples section.

It is to be noted that the compound of the present invention may have other beneficial properties in addition to or in the alternative to its ability to inhibit HSD activity.

### Therapy

The compounds of the present invention may be used as therapeutic agents—i.e. in therapy applications.

The term “therapy” includes curative effects, alleviation effects, and prophylactic effects.

The therapy may be on humans or animals, preferably female animals or humans, such as female humans.

### Pharmaceutical Compositions

In one aspect, the present invention provides a pharmaceutical composition, which comprises a compound according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Combination Pharmaceutical

The compound of the present invention may be used in combination with one or more other active agents, such as one or more other pharmaceutically active agents.

By way of example, the compounds of the present invention may be used in combination with other 11β-HSD inhibitors and/or other inhibitors such as an aromatase inhibitor (such as for example, 4hydroxyandrostenedione (4-OHA)), and/or a steroid sulphatase inhibitors such as EMATE and/or steroids—such as the naturally occurring sterneurosteroids dehydroepiandrosterone sulfate (DHEAS) and pregnenolone sulfate (PS) and/or other structurally similar organic compounds.

In addition, or in the alternative, the compound of the present invention may be used in combination with a biological response modifier.

The term biological response modifier (“BRM”) includes cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)— such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the compounds of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

### Cancer

The compounds of the present invention may be useful in the method of treatment of cancer.

Cancer remains a major cause of mortality in most Western countries. Cancer therapies developed so far have included blocking the action or synthesis of hormones to inhibit the growth of hormone-dependent tumours. However, more aggressive chemotherapy is currently employed for the treatment of hormone-independent tumours.

Hence, the development of a pharmaceutical for anti-cancer treatment of hormone dependent and/or hormone independent tumours, yet lacking some or all of the side-effects associated with chemotherapy, would represent a major therapeutic advance.

We believe that the compound of the present invention provides a means for the treatment of cancers and, especially, breast cancer.

In addition or in the alternative the compound of the present invention may be useful in the blocking the growth of cancers including leukaemias and solid tumours such as breast, endometrium, prostate, ovary and pancreatic tumours.

### Other Therapies

As previously mentioned, in one aspect the present invention provides use of a compound as described herein in the manufacture of a medicament for use in the therapy of a condition or disease associated with 11β-HSD.

Conditions and diseases associated with 11β-HSD have been reviewed in Walker, E. A.; Stewart, P. M.; Trends in Endocrinology and Metabolism, 2003, 14 (7), 334-339.

In a preferred aspect, the condition or disease is selected from the group consisting of:
metabolic disorders, such as diabetes and obesity
cardiovascular disorders, such as hypertension
glaucoma
inflammatory disorders, such as arthritis or asthma
immune disorders
bone disorders, such as osteoporosis
cancer
intra-uterine growth retardation
apparent mineralocorticoid excess syndrome (AME)
polycystic ovary syndrome (PCOS)
hirsutism
acne
oligo- or amenorrhea
adrenal cortical adenoma and carcinoma
Cushing's syndrome
pituitary tumours
invasive carcinomas
wound healing
CNS disorders
breast cancer; and
endometrial cancer.

It is also to be understood that the compound/composition of the present invention may have other important medical implications.

For example, the compound or composition of the present invention may be useful in the treatment of the disorders listed in WO-A-98/05635. For ease of reference, part of that list is now provided: diabetes including Type II diabetes, obesity, cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### SUMMARY

In summation, the present invention provides compounds for use as hydroxysteroid dehydrogenase inhibitors, and pharmaceutical compositions for the same.

The present invention will now be described in further detail in the following examples.

### EXAMPLES

### Experimental Section

### General Procedures for the Synthesis of Amides:

To a solution of the acid in DCM are added EDCI (1.2 eq.), DMAP (catalytic amount) and triethylamine (2 eq.) at room temperature. After 30 minutes, the amine (1 eq) is added to the reaction mixture. After completion, the organic layer is washed with a solution of ammonium chloride and a solution of sodium bicarbonate, dried (MgSO₄) and evaporated under reduce pressure. The crude product is purified with flash chromatography to give the amide.

### Example 1

### 1-[1-(4-Chloro-phenyl)-cyclopropyl]-2-(1-methyl-1H-imidazol-2-ylsulfanyl)-ethanone

2-Mercapto-1-methylimidazole (84 mg, 0.73 mmol) was added neat to a solution of 2-bromo-1-[1-(4-chloro-phenyl)-cyclopropyl]-ethanone (200.5 mg, 0.73 mmol) in CH₃CN (5 mL) at room temperature, then Et₃N (0.203 mL, 1.46 mmol) was added neat to the mixture and the reaction was stirred over night. The reaction was then quenched by addition of a small spatula of resin 2-chlorotrityl chloride, stirred for 1H then the mixture was filtered and concentrated under vacuum. The crude mixture was purified by flash chromatography (hexane/EtOAc gradient 0-50%) to afford the title compound (216.5 mg, 97%) as cream-yellow solid. TLC single spot at R_{f} 0.13 (hexane/EtOAc 7:3); Mp=[95.5-97.0° C.]; ¹H NMR (270 MHz, CDCl₃): δ1.19 (q, J=3.7 Hz, 2H), 1.63 (q, J=3.2 Hz, 2H), 3.62 (s, 3H), 3.86 (s, 2H), 6.87 (d, J=1.2 Hz, 1H), 6.98 (d, J=1.2 Hz, 1H), 7.32 (d, J=0.7 Hz, 4H); LC/MS (APCI) m/z 307 (M⁺+H); HPLC tᵣ=1.88 min (100%) in 10% water-acetonitrile.

### Example 2

### 1-[1-(4-Chloro-phenyl)-cyclopropyl]-2-(1-methyl-1H-imidazole-2-sulfinyl)-ethanone

m-CPBA (77 mg, 0.34 mmol, 60-77% purity) was added neat to a solution of 1-[1-(4-Chloro-phenyl)-cyclopropyl]-2-(1-methyl-1H-imidazol-2-ylsulfanyl)-ethanone (95.9 mg, 0.31 mmol) in dry DCM (5 mL) at −10° C. After 15 minutes the TLC showed no more starting material, so the reaction was quenched by use of a saturated solution of NaHCO₃. The aqueous layer was extracted with DCM, the organic layers were washed with water then brine and dried over MgSO₄ before filtration and concentration. The crude mixture was purified by flash chromatography (hexane/EtOAc gradient 0-70%) to afford the title compound (62.5 mg, 62%) as yellow oil that crystallised. TLC single spot at R_{f} 0.09 (hexane/EtOAc 7:3); Mp=[70.9-73.9° C.]; ¹H NMR (270 MHz, CDCl₃): δ1.23 (d, J=4.2 Hz, 2H), 1.44-1.58 (m, 1H), 1.63-1.77 (m, 1H), 3.88 (s, 3H), 4.19 (d, J=16.0 Hz, 1H), 4.78 (d, J=16.0 Hz, 1H), 6.97 (d, J=1.0 Hz, 1H), 7.14 (d, J=1.0 Hz, 1H), 7.35 (s, 4H); LC/MS (APCI) m/z 323 (M⁺); HPLC tᵣ=1.64 min (>99%) in 10% water-acetonitrile.

### Example 3

### 1-[1-(4-Chloro-phenyl)-cyclopropyl]-2-(1-methyl-1H-imidazole-2-sulfonyl)-ethanone

m-CPBA (141 mg, 0.62 mmol) was added neat to a solution of 1-[1-(4-chloro-phenyl)-cyclopropyl]-2-(1-methyl-1H-imidazol-2-ylsulfanyl)-ethanone (96.5 mg, 0.31 mmol) in dry DCM (5 mL) at 0° C. The reaction was stirred at room temperature for 60 h then was quenched by addition of a saturated solution of NaHCO₃. The aqueous layer was extracted with DCM. The organic layers were washed with water then brine and dried over MgSO₄ before filtration and concentration. The crude mixture was purified by flash chromatography (hexane/EtOAc gradient 0-70%) to give the title compounds (42 mg, 40%) as yellow wet solid. TLC single spot at R_{f} 0.13 (hexane/EtOAc 7:3); Mp=[90.5-95.4° C.]; ¹H NMR (270 MHz, CDCl₃): δ1.25 (q, J=4.0 Hz, 2H), 1.61 (q, J=4.0 Hz, 2H), 3.96 (s, 3H), 4.35 (s, 2H), 6.97 (dd, J=0.7 Hz, 1H), 7.12 (d, J=1.0 Hz, 1H), 7.25-7.37 (m, 4H); LC/MS (APCI) m/z 338 (M⁺−H), 339 (M⁺); HPLC tᵣ=1.72 min (>99%) in 10% water-acetonitrile.

### Example 4

### 1-[1-(4-Chloro-phenyl)-cyclopropyl]-2-(pyridin-2-ylsulfanyl)-ethanone

2-Mercaptopyridine (87 mg, 0.78 mmol) was added neat to a solution of 2-bromo-1-[1-(4-chloro-phenyl)-cyclopropyl]-ethanone (213.5 mg, 0.78 mmol) in DCM (5 mL) at room temperature (20° C.), then Et₃N (0.217 mL, 1.56 mmol) was added neat to the mixture and the reaction was stirred over 48 h. The reaction was quenched by addition of a small spatula of resin 2-chlorotrityl chloride, stirred for 1 h and filtered then concentrated under vacuum. The crude mixture was purified by flash chromatography (hexane/EtOAc gradient 0-20%) to give the expected compound (218.4 mg, 92%) as clear oil. TLC single spot at R_{f} 0.44 (hexane/EtOAc 7:3); ¹H NMR (270 MHz, CDCl₃): δ1.20 (q, J=3.5 Hz, 2H), 1.67 (q, J=3.3 Hz, 2H), 3.91 (s, 2H), 6.93 (ddd, J=7.0, 5.0, 1.0 Hz, 1H), 7.14 (dt, J=8.0, 1.0 Hz, 1H), 7.25-7.33 (m, 2H), 7.37-7.46 (m, 3H), 8.31 (ddd, J=5.0, 1.7, 1.0 Hz, 1H); LC/MS (APCI) m/z 304 (M⁺), 326 (M⁺+Na); HPLC tᵣ=2.58 min (100%) in 10% water-acetonitrile.

### Example 5

### 1-[1-(4-Chloro-phenyl)-cyclopropyl]-2-(pyridine-2-sulfinyl)-ethanone

m-CPBA (64 mg, 0.29 mmol) was added neat to a solution of 1-[1-(4-chloro-phenyl)-cyclopropyl]-2-(pyridin-2-ylsulfanyl)-ethanone (79.5 mg, 0.26 mmol) in dry DCM (5 mL) at −10° C. for 10 min. The reaction was quenched with a saturated solution of NaHCO₃. The aqueous layer was extracted with DCM. The organic layers were washed with water then brine and dried over MgSO₄ before filtration and concentration in vacuum. The crude mixture was purified by flash chromatography (hexane/EtOAc gradient 0-70%) to afford the title compound (77.7 mg, 93%) as white solid. TLC single spot at R_{f} 0.08 (hexane/EtOAc 7:3); Mp=[97.8-99.7° C.]; ¹H NMR (270 MHz, CDCl₃): δ 1.18-1.27 (m, 2H), 1.68-1.79 (m, 2H), 3.76 (d, J=15.3 Hz, 1H), 4.10 (d, J=15.3 Hz, 1H), 7.28 (s, 4H), 7.34 (ddd, J=7.0, 4.7, 2.5 Hz, 1H), 7.84-7.94 (m, 1H), 8.54 (dd, J=7.0, 1.2 Hz, 1H); LC/MS (APCI) m/z 320 (M⁺); HPLC tᵣ=1.78 min (100%) in 10% water-acetonitrile.

### Example 6

### 1-[1-(4-Chloro-phenyl)-cyclopropyl]-2-(pyridine-2-sulfonyl)-ethanone

m-CPBA (125 mg, 0.56 mmol) was added neat to a solution of 1-[1-(4-chloro-phenyl)-cyclopropyl]-2-(pyridin-2-ylsulfanyl)-ethanone (77.1 mg, 0.25 mmol) in dry DCM (5 mL) at 0° C. The reaction was stirred at room temperature for 5 h, then was quenched by addition of a saturated solution of NaHCO₃. The aqueous layer was extracted with DCM. The organic layers were washed with water then brine and dried over MgSO₄ before filtration and evaporation. The crude mixture was purified by flash chromatography (hexane/EtOAc gradient 0-70%) to afford the title compound (75.7 mg, 90%) as white crystalline solid. TLC single spot at R_{f} 0.19 (hexane/EtOAc 7:3); Mp=[116.5-117.4° C.]; ¹H NMR (270 MHz, CDCl₃): δ1.21-1.29 (m, 2H), 1.56-1.67 (m, 2H), 4.45 (s, 2H), 7.29-7.38 (m, 4H), 7.53 (ddd, J=7.4, 4.7, 1.2 Hz, 1H), 7.95 (t, J=7.7, 1.7 Hz, 1H), 8.40 (dt, J=7.7, 1.2 Hz, 1H), 8.68 (ddd, J=4.7, 1.5, 1.0 Hz, 1H); LC/MS (APCI) m/z 358 (M⁺+Na); HPLC tᵣ=1.77 min (98.7%) in 10% water-acetonitrile.

### Example 7

### 1-Adamantan-1-yl-2-(pyridin-3-ylmethylsulfanyl)-ethanone

To a solution of pyridin-3-ylmethyl carbamimidothioate dihydrochloride (480 mg, 2.0 mmol) in water (10 mL) was added NaOH (160 mg). The mixture was stirred at 80° C. under nitrogen for 30 min, cooled to room temperature and diluted with CH₃CN-Et₃N (3 mL:2 mL). After adding 1-adamantyl bromomethyl ketone (514 mg, 2.0 mmol), the mixture was stirred at rt for 6 h, partitioned between DCM and water. The organic phase was washed brine, dried over MgSO₄ and concentrated in vacuo. Purification with flash column (EtOAc-hexane gradient elution) gave product (320 mg, 53%) as off-white solid. mp 45-47° C.; TLC single spot at R_{f}: 0.51 (40% EtOAc/hexane); ¹H NMR (270 MHz, CDCl₃) δ 1.62-1.75 (6H, m, 3×CH₂), 1.80 (6H, d, J=2.7 Hz, 3×CH₂), 2.02 (3H, broad, 3×CH), 3.21 (2H, s, CH₂), 3.71 (2H, s, CH₂), 7.24 (1H, dd, J=7.7, 4.7 Hz, ArH), 7.69 (1H, dt, J=7.7, 1.8 Hz, ArH), 8.49 (1H, dd, J=4.7, 1.7 Hz, ArH) and 8.53 (1H, d, J=2.2 Hz, ArH); LC/MS (ESI) m/z 302 (M⁺+H), tᵣ=1.36 min in 5% water-methanol; HRMS (ESI) calcd. for C₁₈H₂₄NOS (M⁺+H) 302.1579. found 302.1583; HPLC tᵣ=2.75 min (99%) in 10% water-acetonitrile.

### Example 8

### 1-Adamantan-1-yl-2-(pyridin-3-ylmethanesulfinyl)-ethanone

To a cold solution of 1-adamantan-1-yl-2-(pyridin-3-ylmethylsulfanyl)-ethanone (260 mg, 0.86 mmol) in DCM (25 mL) was added mCPBA (230 mg, purity 60-77%). The mixture was stirred at −5° C. for 30 min, partitioned between DCM and 5% sodium carbonate solution. The organic phase was washed with brine, dried over MgSO₄ and concentrated in vacuo to give the crude product. Purification with flash column (methanol-DCM; gradient elution) yielded the title compound as white solid (250 mg, 92%). mp 116-119° C.; TLC single spot at R_{f}: 0.25 (40% EtOAc/hexane); ¹H NMR (270 MHz, CDCl₃) δ 1.62-1.81 (12H, m, 6×CH₂), 2.05 (3H, broad, 3×CH), 3.56 (1H, d, J=16 Hz, CH), 3.93 (1H, d, J=16 Hz, CH), 4.02 (1H, d, J=14 Hz, CH), 4.25 (1H, d, J=14 Hz, CH), 7.32 (1H, dd, J=7.9, 4.9 Hz, ArH), 7.69 (1H, dt, J=7.9, 2.2 Hz, ArH), 8.49 (1H, d, J=1.9 Hz, ArH) and 8.61 (1H, dd, J=4.9, 1.7 Hz, ArH); LC/MS (ESI) m/z 316 (M⁺−H); tᵣ=1.00 min in 5% water-methanol; HRMS (ESI) calcd. for C₁₈H₂₄NO₂S (M⁺+H) 318.1528. found 318.1514; HPLC tᵣ=1.78 min (>99%) in 10% water-acetonitrile.

### Example 9

### 1-Adamantan-1-yl-2-(pyridin-3-ylmethanesulfonyl)-ethanone and

### Example 10

### 1-Adamantan-1-yl-2-(1-oxy-pyridin-3-ylmethanesulfonyl)-ethanone

To a solution of 1-adamantan-1-yl-2-(pyridin-3-ylmethanesulfinyl)-ethanone (130 mg, 0.41 mmol) in DCM (5 mL) was added mCPBA (110 mg, purity 60-77%). The mixture was stirred at rt overnight, partitioned between DCM and 5% sodium carbonate solution. The organic phase was washed with brine, dried over MgSO₄ and concentrated in vacuo to give the crude product. Purification with flash column (methanol-DCM; gradient elution) yielded the Example 9 as white solid (46 mg, 34%). mp 150-151° C.; TLC single spot at R_{f}: 0.76 (10% CH₃OH/DCM); ¹H NMR (270 MHz, CDCl₃) δ 1.63-1.73 (6H, m, 3×CH₂), 1.79 (6H, d, J=2.7 Hz, 3×CH₂), 2.08 (3H, broad, 3×CH), 3.89 (2H, s, CH₂), 4.54 (2H, s, CH₂), 7.34 (1H, dd, J=7.9, 5.0 Hz, ArH), 7.85 (1H, dt, J=7.9, 1.7 Hz, ArH), 8.63 (1H, dd, J=5.0, 1.7 Hz, ArH) and 8.68 (1H, d, J=2.0 Hz, ArH); LC/MS (ESI) m/z 334 (M⁺+H); tᵣ=1.05 min in 5% water-methanol; HRMS (ESI) calcd. for C₁₈H₂₄NO₃S (M⁺+H) 334.1477. found 334.1475; HPLC tᵣ=1.93 min (>99%) in 10% water-acetonitrile.

Example 10 was obtained as white solid (80 mg, 56%). mp 182-183.5° C.; TLC single spot at R_{f}: 0.52 (10% CH₃OH/DCM); ¹H NMR (270 MHz, CDCl₃) δ 1.61-1.74 (6H, m, 3×CH₂), 1.80 (6H, d, J=2.7 Hz, 3×CH₂), 2.09 (3H, broad, 3×CH), 3.96 (2H, s, CH₂), 4.47 (2H, s, CH₂), 7.24-7.42 (2H, m, ArH), 8.20 (1H, dt, J=6.4, 1.7 Hz, ArH) and 8.36 (1H, br, ArH); LC/MS (ESI) m/z 350 (M⁺+H); tᵣ=1.01 min in 5% water-methanol; HRMS (FAB+) calcd. for C₁₈H₂₄NO₄S (M⁺+H) 350.1426. found 350.1410; HPLC tᵣ=1.62 min (>99%) in 10% water-acetonitrile.

### Example 11

### 1-Adamantan-1-yl-2-(pyridin-2-ylmethylsulfanyl)-ethanone

To a solution of pyridin-2-ylmethyl carbamimidothioate dihydrochloride (480 mg, 2.0 mmol) in water (10 mL) was added NaOH (160 mg). The mixture was stirred at 80° C. under nitrogen for 45 min, cooled to room temperature and diluted with CH₃CN-Et₃N (3 mL:2 mL). After adding 1-adamantyl bromomethyl ketone (514 mg, 2.0 mmol), the mixture was stirred at rt overnight, partitioned between DCM and water. The organic phase was washed brine, dried over MgSO₄ and concentrated in vacuo. Purification with flash column (EtOAc-hexane gradient elution) gave product (550 mg, 91%) as off-white solid. mp 38-39° C.; TLC single spot at R_{f}: 0.49 (50% EtOAc/hexane); ¹H NMR (270 MHz, CDCl₃) δ 1.61-1.75 (6H, m, 3×CH₂), 1.81 (6H, d, J=2.7 Hz, 3×CH₂), 2.02 (3H, broad, 3×CH), 3.38 (2H, s, CH₂), 3.83 (2H, s, CH₂), 7.15 (1H, ddd, J=7.6, 4.8, 1.0 Hz, ArH), 7.36 (1H, dt, J=7.8; 1.0 Hz, ArH), 7.63 (1H, td, J=7.6, 1.7 Hz, ArH) and 8.54 (1H, dq, J=5.0, 1.0 Hz, ArH); LC/MS (ESI) m/z 302 (M⁺+H), tᵣ=1.31 min in 5% water-methanol; HRMS (ESI) calcd. for C₁₈H₂₄NOS (M⁺+H) 302.1579. found 302.1585; HPLC tᵣ=2.82 min (99%) in 10% water-acetonitrile.

### Example 12

### 1-Adamantan-1-yl-2-(pyridin-2-ylsulfanyl)-ethanone

To a solution of adamantan-1-yl bromomethyl ketone (514 mg, 2.0 mmol) in acetonitrile (8 mL) was added pyridine-2-thiol (244 mg, 2.1 mmol), followed by triethylamine (1 mL). The mixture was stirred at ambient temperature overnight, partitioned between ethyl acetate and saturated sodium carbonate. The organic phase was washed with brine, dried over sodium sulfate and concentrated in vacuo to give the crude product. Purification with flash column (hexane-ethyl acetate; gradient elution) yielded the title compound as white solid (570 mg, 99%). mp 60-61° C.; TLC single spot at R_{f}: 0.69 (20% hexane/DCM); ¹H NMR (270 MHz, CDCl₃) δ 1.68-1.79 (6H, m, 3×CH₂), 1.94 (6H, d, J=2.7 Hz, 3×CH₂), 2.07 (3H, broad, 3×CH), 4.23 (2H, s, CH₂), 6.93 (1H, ddd, J=7.4, 4.9, 1.0 Hz, ArH), 7.21 (1H, dt, J=8.2, 1.0 Hz, ArH), 7.44 (1H, ddd, J=9.2, 7.4, 2.0 Hz, ArH) and 8.32 (1H, dq, J=4.9, 1.0 Hz, ArH); LC/MS (ESI) m/z 288 (M⁺+H); tᵣ=1.47 min in 5% water-methanol; HRMS (ESI) calcd. for C₁₇H₂₂NOS (M⁺+H) 288.1422. found 288.1431; HPLC tᵣ=3.60 min (>99%) in 10% water-acetonitrile.

### Example 13

### 1-Adamantan-1-yl-2-(pyrimidin-2-ylsulfanyl)-ethanone

To a solution of adamantan-1-yl bromomethyl ketone (514 mg, 2.0 mmol) in acetonitrile (8 mL) was added pyrimidine-2-thiol (249 mg, 2.1 mmol), followed by triethylamine (1 mL). The mixture was stirred at ambient temperature overnight, partitioned between ethyl acetate and saturated sodium carbonate. The organic phase was washed with brine, dried over sodium sulfate and concentrated in vacuo to give the crude product. Purification with flash column (hexane-ethyl acetate; gradient elution) yielded the title compound as white solid (510 mg, 88%). mp 100-101° C.; TLC single spot at R_{f}: 0.28 (20% hexane/DCM); ¹H NMR (270 MHz, CDCl₃) δ 1.69-1.82 (6H, m, 3×CH₂), 1.94 (6H, d, J=2.7 Hz, 3×CH₂), 2.07 (3H, broad, 3×CH), 4.19 (2H, s, CH₂), 6.93 (1H, t, J=4.9 Hz, ArH), and 8.45 (2H, d, J=4.9 Hz, ArH);

LC/MS (ESI) m/z 311 (M⁺+Na), tᵣ=1.02 min in 5% water-methanol; HRMS (ESI) calcd. for C₁₆H₂₁N₂OS (M⁺+H) 289.1375. found 289.1360; HPLC tᵣ=2.73 min (>99%) in 10% water-acetonitrile.

### Example 14

### 1-Adamantan-1-yl-2-(1-methyl-1H-benzoimidazol-2-ylsulfanyl)-ethanone

To a solution of adamantan-1-yl bromomethyl ketone (514 mg, 2.0 mmol) in acetonitrile (8 mL) was added 1-methyl-1H-benzo[d]imidazole-2-thiol (345 mg, 2.1 mmol), followed by triethylamine (1 mL). The mixture was stirred at ambient temperature overnight, partitioned between ethyl acetate and saturated sodium carbonate. The organic phase was washed with brine, dried over sodium sulfate and concentrated in vacuo to give the crude product. Purification with flash column (hexane-ethyl acetate; gradient elution) yielded the title compound as white solid (580 mg, 85%). mp 146-147.5° C.; TLC single spot at R_{f}: 0.65 (40% hexane/DCM); ¹H NMR (270 MHz, CDCl₃) δ 1.68-1.79 (6H, m, 3×CH₂), 1.93 (6H, d, J=2.7 Hz, 3×CH₂), 2.07 (3H, broad, 3×CH), 3.72 (3H, s, CH₃), 4.57 (2H, s, CH₂), 7.16-7.26 (3H, m, ArH), and 7.61 (1H, m, ArH); LC/MS (ESI) m/z 341 (M⁺+H), tᵣ=1.26 min in 5% water-methanol; HRMS (ESI) calcd. for C₂₀H₂₅N₂OS (M⁺+H) 341.1688. found 341.1674; HPLC tᵣ=3.16 min (>99%) in 10% water-acetonitrile.

### Example 15

### 1-Adamantan-1-yl-2-methyl-2-(1-methyl-1H-imidazole-2-sulfinyl)-propan-1-one

To a solution 1-adamantan-1-yl-2-(1-methyl-1H-imidazole-2-sulfinyl)-ethanone (109 mg, 0.35 mmol) in THF (5 mL) was added NaH (70 mg, 60% dispersion), followed by CH₃I (0.11 mL, 1.75 mmol) and DMF (0.1 mL). The mixture was stirred at rt overnight, partitioned between EtOAc and water. The organic phase was washed brine, dried over MgSO₄ and concentrated in vacuo. Purification with flash column (CH₃OH-DCM gradient elution) gave product (65 mg, 56%) as off-white solid. mp 98-102. ° C.; TLC single spot at R_{f}: 0.21 (6% CH₃OH/DCM); ¹H NMR (270 MHz, CDCl₃) δ 1.60-1.69 (6H, m, 3×CH₂), 1.73 (3H, s, CH₃), 1.74-1.87 (6H, m, 3×CH₂), 1.98 (3H, broad, 3×CH), 2.02 (3H, s, CH₃), 3.88 (3H, s, CH₃), 6.89 (1H, d, J=1.0 Hz, ArH) and 7.08 (1H, d, J=1.0 Hz, ArH); LC/MS (ESI) m/z 335 (M⁺+H), tᵣ=1.14 min in 5% water-methanol; HRMS (ESI) calcd. for C₁₈H₂₇N₂O₂S (M⁺+H) 335.1793. found 335.1775; HPLC tᵣ=2.51 min (92%) in 10% water-acetonitrile.

### Example 16

### 1-Adamantan-1-yl-2-(pyridin-2-ylmethanesulfonyl)-ethanone and

### Example 17

### 1-Adamantan-1-yl-2-(pyridin-2-ylmethanesulfinyl)-ethanone

To a cold (−5° C.) solution of 1-adamantan-1-yl-2-(pyridin-2-ylmethanesulfinyl)-ethanone (400 mg, 1.33 mmol) in DCM (30 mL) was added mCPBA (357 mg, purity 60-77%). The mixture was stirred at −5° C. for 1 h, partitioned between DCM and 5% sodium carbonate solution. The organic phase was washed with brine, dried over MgSO₄ and concentrated in vacuo to give the crude product. Purification with flash column (methanol-DCM; gradient elution) yielded the Example 16 as white solid 50 mg, 11%). mp 115.5-117° C.; TLC single spot at R_{f}: 0.87 (5% CH₃OH/DCM); ¹H NMR (270 MHz, CDCl₃) δ 1.64-1.78 (6H, m, 3×CH₂), 1.83 (6H, d, J=2.7 Hz, 3×CH₂), 2.07 (3H, broad, 3×CH), 4.26 (2H, s, CH₂), 4.70 (2H, s, CH₂), 7.28 (1H, ddd, J=7.7, 4.9, 1.2 Hz, ArH), 7.42 (1H, d, J=7.7 Hz, ArH), 7.72 (1H, td, J=7.6, 2.0 Hz, ArH) and 8.59 (1H, dq, J=4.9, 0.8 Hz, ArH); LC/MS (ESI) m/z 332 (M⁺−H); tᵣ=1.00 min in 5% water-methanol; HRMS (ESI) calcd. for C₁₈H₂₄NO₃S (M⁺+H) 334.1477. found 334.1470; HPLC tᵣ=2.13 min (>99%) in 10% water-acetonitrile.

Example 17 was obtained as white solid (220 mg, 52%). mp 87-88.5° C.; TLC single spot at R_{f}: 0.52 (10% CH₃OH/DCM); ¹H NMR (270 MHz, CDCl₃) δ 1.69-1.79 (6H, m, 3×CH₂), 1.80 (6H, d, J=2.7 Hz, 3×CH₂), 2.05 (3H, broad, 3×CH), 3.87 (1H, d, J=15.9 Hz, CH), 4.02 (1H, d, J=15.9 Hz, CH), 4.19 (1H, d, J=12.9 Hz, CH), 4.36 (1H, d, J=12.9 Hz, CH), 7.25 (1H, ddd, J=7.6, 4.9, 0.9 Hz, ArH), 7.35 (1H, d, J=7.7 Hz, ArH), 7.70 (1H, td, J=7.6, 1.7 Hz, ArH) and 8.60 (1H, dq, J=5.0, 0.7 Hz, ArH); LC/MS (ESI) m/z 340 (M⁺+Na); tᵣ=1.09 min in 5% water-methanol; HRMS (FAB+) calcd. for C₁₈H₂₄NO₂S (M⁺+H) 318.1528. found 318.1521; HPLC tᵣ=1.93 min (>99%) in 10% water-acetonitrile.

### Example 18

### 1-Adamantan-1-yl-2-(pyridine-2-sulfonyl)-ethanone and

### Example 19

### 1-Adamantan-1-yl-2-(pyridine-2-sulfinyl)-ethanone

To a cold (−5° C.) solution of 1-adamantan-1-yl-2-(pyridin-2-ylsulfanyl)-ethanone (409 mg, 1.43 mmol) in DCM (30 mL) was added mCPBA (394 mg, purity 60-77%). The mixture was stirred at −5° C. for 1 h, partitioned between DCM and 5% sodium carbonate solution. The organic phase was washed with brine, dried over MgSO₄ and concentrated in vacuo to give the crude product. Purification with flash column (EtOAc-DCM; gradient elution) yielded the Example 18 as white solid (24 mg, 5%). mp 129-131° C.; TLC single spot at R_{f}: 0.70 (30% EtOAc/DCM); ¹H NMR (270 MHz, CDCl₃) δ 1.54-1.76 (12H, m, 6×CH₂), 2.04 (3H, broad, 3×CH), 4.67 (2H, s, CH₂), 7.53 (1H, ddd, J=7.8, 4.9, 1.8 Hz, ArH), 7.97 (1H, td, J=7.7, 1.8 Hz, ArH), 8.08 (1H, dt, J=8.0, 1.0 Hz, ArH) and 8.70 (1H, dq, J=5.0, 0.8 Hz, ArH); LC/MS (ESI) m/z 318 (M⁺−H); tᵣ=0.97 min in 5% water-methanol; HRMS (ESI) calcd. for C₁₇H₂₂NO₃S (M⁺+H) 320.1320. found 320.1315; HPLC tᵣ=2.14 min (>99%) in 10% water-acetonitrile.

Example 19 was obtained as white solid (368 mg, 85%). mp 66-68° C.; TLC single spot at R_{f}: 0.60 (30% EtOAc/DCM); ¹H NMR (270 MHz, CDCl₃) δ 1.58-1.76 (6H, m, 3×CH₂), 1.81 (6H, d, J=2.7 Hz, 3×CH₂), 2.04 (3H, broad, 3×CH), 4.05 (1H, d, J=15.6 Hz, CH), 4.32 (1H, d, J=15.6 Hz, CH), 7.38 (1H, m, ArH), 7.90-8.20 (2H, m, ArH) and 8.61 (1H, dq, J=5.1, 0.8 Hz, ArH); LC/MS (ESI) m/z 302 (M⁺−H); tᵣ=1.03 min in 5% water-methanol; HRMS (FAB+) calcd. for C₁₇H₂₂NO₂S (M+H) 304.1371. found 304.1366; HPLC tᵣ=2.17 min (>99%) in 10% water-acetonitrile.

### Example 20

### 1-[1-(4-Chloro-phenyl)-cyclobutyl]-2-(pyridin-2-ylsulfanyl)-ethanone

2-Mercaptopyridine (42 mg, 0.38 mmol) was added neat to a solution of 2-bromo-1-[1-(4-chloro-phenyl)-cyclobutyl]-ethanone (110 mg, 0.38 mmol) in CH₃CN (5 mL) at room temperature, then Et₃N (0.107 mL, 0.76 mmol) was added neat to the mixture and the reaction was stirred overnight. The traces of thiol left were quenched by addition of a small spatula of resin 2-chlorotrityl chloride, stirred for 1 h then the mixture was filtered and concentrated under vacuum. The crude mixture was purified by flash chromatography (hexane/EtOAc gradient 0-30%) to give the expected compound (97 mg, 80.6%) as yellow oil. TLC single spot at R_{f} 0.43 (hexane/EtOAc 7:3); ¹H NMR (270 MHz, CDCl₃): δ1.79-2.04 (m, 2H), 2.38-2.52 (m, 2H), 2.90-3.02 (m, 2H), 3.87 (s, 2H), 6.93 (ddd, J=7.4, 5.0, 1.2 Hz, 1H), 7.14 (dt, J=8.0, 1.0 Hz, 1H), 7.20-7.27 (m, 3H), 7.29-7.36 (m, 2H), 7.42 (ddd, J=8.0, 7.3, 2.0 Hz, 1H), 8.21 (ddd, J=5.0, 2.0, 1.0 Hz, 1H); LC/MS (APCI) m/z 318 (M⁺+H), 340 (M⁺+Na); HPLC tᵣ=3.15 min (>99%) in 10% water-acetonitrile.

### Example 21

### 1-[1-(4-Chloro-phenyl)-cyclobutyl]-2-(1-methyl-1H-imidazol-2-ylsulfanyl)-ethanone

2-Mercapto-1-methylimidazole (83 mg, 0.73 mmol) was added neat to a solution of 2-bromo-1-[1-(4-chloro-phenyl)-cyclobutyl]-ethanone (210 mg, 0.73 mmol) in CH₃CN (7 mL) at room temperature, then Et₃N (0.203 mL, 1.46 mmol) was added neat to the mixture and the reaction was stirred overnight and over the next 4 days. The traces of thiol left were quenched by addition of a small spatula of resin 2-chlorotrityl chloride, stirred for 1 h then the mixture was filtered and concentrated under vacuum. The crude mixture was purified by flash chromatography (hexane/EtOAc gradient 0-50%) to give the expected compound (179 mg, 76%) as yellow oil. TLC single spot at R_{f} 0.18 (hexane/EtOAc 5:5); ¹H NMR (270 MHz, CDCl₃): δ1.74-1.92 (m, 2H), 2.29-2.44 (m, 2H), 2.69-2.84 (m, 2H), 3.59 (s, 3H), 3.84 (s, 2H), 6.85 (d, J=1.2 Hz, 1H), 6.96 (d, J=1.2 Hz, 1H), 7.13 (dt, J=8.7, 2.5 Hz, 2H), 7.29 (dt, J=8.7, 2.5 Hz, 2H); LC/MS (APCI) m/z 321 (M⁺+H); Accurate Mass: Calculated (M+H)⁺ 321.0823. Found 321.0824; HPLC tᵣ=2.15 min (100%) in 10% water-acetonitrile.

### Example 22

### 1-[1-(4-Chloro-phenyl)-cyclobutyl]-2-(thiophen-2-ylmethanesulfonyl)-ethanone

### Example 23

### 1-[1-(4-Chloro-phenyl)-cyclobutyl]-2-(thiophen-2-ylmethanesulfinyl)-ethanone

m-CPBA (85 mg, 0.38 mmol) was added neat to a solution of 1-[1-(4-chloro-phenyl)-cyclobutyl]-2-(pyridin-2-ylsulfanyl)-ethanone (60 mg, 0.19 mmol) in dry DCM (5 mL) at 0° C. over night. The reaction was quenched by addition of a saturated solution of NaHCO₃. The aqueous layer was extracted with DCM, the organic layers were washed with water then brine and dried over MgSO₄ before filtration and concentration in vacuum. The crude mixture was purified by flash chromatography (hexane/EtOAc gradient 0-40%) to give the expected sulfone Example 22 (50.4 mg, 72%) as white solid and the sulfoxide Example 23 (33.1 mg, 49%) as white solid.

### Example 22

TLC single spot at R_{f} 0.18 (hexane/EtOAc 7:3); Mp=[88.4-90.6° C.]; ¹H NMR (270 MHz, CDCl₃): δ 1.78-1.94 (m, 2H), 2.28-2.42 (m, 2H), 2.71-2.85 (m, 2H), 4.37 (s, 2H), 7.08 (dt, J=8.7, 2.7 Hz, 2H), 7.32 (dt, J=8.9, 2.5 Hz, 2H), 7.53 (ddd, J=7.4, 4.7, 1.2 Hz, 1H), 7.96 (td, J=7.5, 1.7 Hz, 1H), 8.07 (dt, J=8.0, 1.0 Hz, 1H), 8.61 (ddd, J=4.7, 1.7, 1.0 Hz, 1H); LC/MS (APCI) 348 (M⁺−H); HPLC tᵣ=2.06 min (100%) in 10% water-acetonitrile.

### Example 23

TLC single spot at R_{f} 0.09 (hexane/EtOAc 7:3); Mp=[121.9-123.3° C.]; ¹H NMR (270 MHz, CDCl₃): δ 1.78-2.04 (m, 2H), 2.24-2.48 (m, 2H), 2.72-2.98 (m, 2H), 3.72 (d, J=15.1 Hz, 1H), 3.98 (d, J=15.3 Hz, 1H), 7.10 (d br, J=8.6 Hz, 2H), 7.25-7.37 (m, 3H), 7.85-7.97 (m, 2H), 8.57 (qd, J=4.7, 0.7 Hz, 1H); LC/MS (APCI) m/z 356 (M⁺+Na); Accurate Mass: Calculated (M⁺+Na) 356.0482. Found 356.0484; HPLC tᵣ=2.14 min (98.6%) in 10% water-acetonitrile.

### Example 24

### 1-[1-(4-Chloro-phenyl)-cyclobutyl]-2-(1-methyl-1H-imidazole-2-sulfonyl)-ethanone

### Example 25

### 1-[1-(4-Chloro-phenyl)-cyclobutyl]-2-(1-methyl-1H-imidazole-2-sulfinyl)-ethanone

m-CPBA (81 mg, 0.36 mmol) was added neat to a solution of 1-[1-(4-chloro-phenyl)-cyclobutyl]-2-(1-methyl-1H-imidazol-2-ylsulfanyl)-ethanone (58.3 mg, 0.18 mmol) in dry DCM (5 mL) at 0° C. over night. The reaction was quenched by addition of a saturated solution of NaHCO₃. The aqueous layer was extracted with DCM, the organic layers were washed with water then brine and dried over MgSO₄ before filtration and concentration in vacuum. The crude mixture was purified by flash chromatography (DCM/MeOH gradient 0-10%) to give the expected sulfone Example 24 (26.5 mg, 42%) as white solid and the sulfoxide Example 25 (33.6 mg, 55%) as white yellow oil.

### Example 24

TLC single spot at R_{f} 0.83 (DCM/MeOH 9:1); Mp=[94.5-96.5° C.]; ¹H NMR (270 MHz, CDCl₃): δ 1.76-1.92 (m, 2H), 2.28-2.44 (m, 2H), 2.68-2.82 (m, 2H), 4.01 (s, 3H), 4.29 (s, 2H), 6.99 (d, J=0.7 Hz, 1H), 7.07 (dt, J=8.6, 2.5 Hz, 2H), 7.12 (d, J=1.0 Hz, 1H), 7.32 (dt, J=8.6, 2.7 Hz, 2H); LC/MS (APCI) m/z 353 (M⁺); HPLC tᵣ=1.95 min (>99%) in 10% water-acetonitrile.

### Example 25

TLC single spot at R_{f} 0.34 (DCM/MeOH 9:1); ¹H NMR (270 MHz, CDCl₃): δ1.76-1.94 (m, 2H), 2.27-2.47 (m, 2H), 2.72-2.89 (m, 2H), 3.88 (s, 3H), 4.17 (d, J=15.8 Hz, 1H), 4.60 (d, J=16.0 Hz, 1H), 6.97 (d, J=1.0 Hz, 1H), 7.09 (d, J=1.0 Hz, 1H), 7.14 (dt, J=8.6, 2.5 Hz, 2H), 7.32 (dt, J=8.9, 2.5 Hz, 2H); LC/MS (APCI) m/z 337 (M⁺); HPLC tᵣ=2.93 min (>91% purity) in 30% water-methanol.

### Example 26

### 1-Adamantan-1-yl-2-(6-methyl-pyridin-2-ylsulfanyl)-ethanone

To a solution of adamantan-1-yl bromomethyl ketone (514 mg, 2.0 mmol) in acetonitrile (10 mL) was added 6-methylpyridine-2-thiol (275 mg, 2.2 mmol), followed by triethylamine (1 mL). The mixture was stirred at ambient temperature overnight, partitioned between ethyl acetate and saturated sodium carbonate. The organic phase was washed with brine, dried over MgSO₄ and concentrated in vacuo to give the crude product. Purification with flash column (hexane-ethyl acetate; gradient elution) yielded the title compound as white solid (538 mg, 89%). mp 115.5-116.5° C.

TLC single spot at R_{f}: 0.75 (25% EtOAc/hexane); ¹H NMR (270 MHz, CDCl₃) δ 1.68-1.82 (6H, m, 3×CH₂), 1.95 (6H, d, J=2.7 Hz, 3×CH₂), 2.07 (3H, broad, 3×CH), 2.43 (3H, s, CH₃), 4.22 (2H, s, CH₂), 6.78 (1H, d, J=7.9 Hz, ArH), 7.03 (1H, d, J=7.9 Hz, ArH) and 7.33 (1H, t, J=7.9 Hz, ArH); LC/MS (ESI) m/z 302 (M⁺+H); tᵣ=1.60 min in 5% water-methanol; HRMS (ESI) calcd. for C₁₈H₂₄NOS (M⁺+H) 302.1579. found 302.1583; HPLC tᵣ=4.45 min (>99%) in 10% water-acetonitrile.

### Example 27

### 1-Adamantan-1-yl-2-(pyridin-4-ylsulfanyl)-ethanone

To a solution of adamantan-1-yl bromomethyl ketone (514 mg, 2.0 mmol) in acetonitrile (10 mL) was added pyridine-4-thiol (244 mg, 2.2 mmol), followed by triethylamine (1 mL). The mixture was stirred at ambient temperature overnight, partitioned between ethyl acetate and saturated sodium carbonate. The organic phase was washed with brine, dried over MgSO₄ and concentrated in vacuo to give the crude product. Purification with flash column (hexane-ethyl acetate; gradient elution) yielded the title compound as white solid (494 mg, 86%). mp 114-115° C.; TLC single spot at R_{f}: 0.32 (25% EtOAc/DCM); ¹H NMR (270 MHz, CDCl₃) δ 1.70-1.82 (6H, m, 3×CH₂), 1.90 (6H, d, J=2.8 Hz, 3×CH₂), 2.08 (3H, broad, 3×CH), 4.01 (2H, s, CH₂), 7.08 (2H, dd, J=4.7, 1.8 Hz, ArH) and 8.38 (2H, dd, J=4.7, 1.8 Hz, ArH); LC/MS (ESI) m/z 288 (M⁺+H); tᵣ=1.12 min in 5% water-methanol; HRMS (ESI) calcd. for C₁₇H₂₂NOS (M⁺+H) 288.1422. found 288.1420; HPLC tᵣ=2.64 min (>99%) in 10% water-acetonitrile.

### Example 28

### 1-Adamantan-1-yl-2-(4-methyl-4H-[1,2,4]triazol-3-ylsulfanyl)-ethanone

To a solution of adamantan-1-yl bromomethyl ketone (514 mg, 2.0 mmol) in acetonitrile (10 mL) was added 4-methyl-4H-1,2,4-triazole-3-thiol (253 mg, 2.2 mmol), followed by triethylamine (1 mL). The mixture was stirred at ambient temperature overnight, partitioned between ethyl acetate and saturated sodium carbonate. The organic phase was washed with brine, dried over MgSO₄ and concentrated in vacuo to give the crude product. Purification with flash column (hexane-ethyl acetate; gradient elution) yielded the title compound as white solid (340 mg, 58%). mp 134.5-135.5° C.; TLC single spot at R_{f}: 0.22 (10% CH₃OH/DCM); ¹H NMR (270 MHz, CDCl₃) δ 1.60-1.80 (6H, m, 3×CH₂), 1.87 (6H, d, J=2.8 Hz, 3×CH₂), 2.05 (3H, broad, 3×CH), 3.63 (3H, s, CH₃), 4.47 (2H, s, CH₂) and 8.09 (1H, s, ArH); LC/MS (ESI) m/z 292 (M⁺+H); tᵣ=1.69 min in 5% water-methanol; HRMS (ESI) calcd. for C₁₅H₂₂N₃OS (M⁺+H) 292.1484. found 292.1484; HPLC tᵣ=1.96 min (>99%) in 10% water-acetonitrile.

### Example 29

### 6-(2-Adamantan-1-yl-2-oxo-ethylsulfanyl)-nicotinic acid

To a solution of adamantan-1-yl bromomethyl ketone (514 mg, 2.0 mmol) in acetonitrile (10 mL) was added 6-mercaptonicotinic acid (326 mg, 2.1 mmol), followed by triethylamine (1 mL). The mixture was stirred at ambient temperature overnight, diluted with water, neutralized with 4N HCl and extracted with DCM. The organic phase was washed with brine, dried over MgSO₄ and concentrated in vacuo to give the crude product. Purification with flash column (CH₃OH-DCM; gradient elution) yielded the title compound as white solid (430 mg, 65%). mp 174-176° C.

TLC single spot at R_{f}: 0.46 (50% EtOAc/DCM); ¹H NMR (270 MHz, CDCl₃) δ 1.65-1.80 (6H, m, 3×CH₂), 1.95 (6H, d, J=2.7 Hz, 3×CH₂), 2.00 (3H, broad, 3×CH), 4.28 (2H, s, CH₂), 7.31 (1H, dd, J=8.6, 0.7 Hz, ArH), 8.06 (1H, dd, J=8.6, 2.2 Hz, ArH) and 8.97 (1H, dd, J=2.2, 0.7 Hz, ArH); LC/MS (ESI) m/z 332 (M⁺+H); tᵣ=1.15 min in 5% water-methanol; HRMS (ESI) calcd. for C₁₈H₂₂NO₃S (M⁺+H) 332.1320. found 332.1302; HPLC tᵣ=1.57 min (95%) in 10% water-acetonitrile.

### Example 30

### 1-(adamantan-1-yl)-2-[(6-methylpyridine-2-)sulfonyl]ethan-1-one and

### Example 31

### 1-(adamantan-1-yl)-2-[(6-methylpyridine-2-)sulfinyl]ethan-1-one

To a cold (−5° C.) solution of 1-adamantan-1-yl-2-(6-methyl-pyridin-2-ylsulfanyl)-ethanone (373 mg, 1.24 mmol) in DCM (30 mL) was added mCPBA (342 mg, purity 60-77%). The mixture was stirred at −5° C. for 1 h, partitioned between DCM and 5% sodium carbonate solution. The organic phase was washed with brine, dried over MgSO₄ and concentrated in vacuo to give the crude product. Purification with flash column (methanol-DCM; gradient elution) yielded the Example 30 as white solid (33 mg, 8%). mp 151-152° C.; TLC single spot at R_{f}: 0.70 (25% EtOAc/DCM); ¹H NMR (270 MHz, CDCl₃) δ 1.56-1.72 (6H, m, 3×CH₂), 1.77 (6H, d, J=2.7 Hz, 3×CH₂), 2.05 (3H, broad, 3×CH), 2.60 (3H, s, CH₃), 4.67 (2H, s, CH₂), 7.35 (1H, d, J=7.5 Hz, ArH), 7.82 (1H, t, J=7.4 Hz, ArH) and 7.89 (1H, d, J=7.6 Hz, ArH); LC/MS (ESI) m/z 334 (M⁺+H); tᵣ=1.06 min in 5% water-methanol; HRMS (ESI) calcd. for C₁₈H₂₄NO₃S (M⁺+H) 334.1477. found 334.1458; HPLC tᵣ=2.45 min (>99%) in 10% water-acetonitrile.

Example 31 was obtained as white solid (270 mg, 69%). mp 75-76° C.; TLC single spot at R_{f}: 0.55 (25% EtOAc/DCM); ¹H NMR (270 MHz, CDCl₃) δ 1.59-1.76 (6H, m, 3×CH₂), 1.81 (6H, d, J=2.7 Hz, 3×CH₂), 2.04 (3H, broad, 3×CH), 2.57 (3H, s, CH₃), 4.03 (1H, d, J=15.4 Hz, CH), 4.26 (1H, d, J=15.4 Hz, CH), 7.21 (1H, t, J=4.4 Hz, ArH) and 7.80 (2H, d, J=4.4 Hz, ArH); LC/MS (ESI) m/z 318 (M⁺+H); tᵣ=1.11 min in 5% water-methanol; HRMS (ESI) calcd. for C₁₈H₂₄NO₃S (M⁺+H) 334.1477. found 334.1458; HPLC tᵣ=2.48 min (>99%) in 10% water-acetonitrile.

### Example 32

### 1-(adamantan-1-yl)-2-(pyridine-4-sulfonyl)ethan-1-one and Example 33: 1-(adamantan-1-yl)-2-(pyridine-4-sulfinyl)ethan-1-one

To a cold (−5° C.) solution of 1-adamantan-1-yl-2-(pyridin-4-ylsulfanyl)-ethanone (370 mg, 1.29 mmol) in DCM (30 mL) was added mCPBA (355 mg, purity 60-77%). The mixture was stirred at −5° C. for 1 h, partitioned between DCM and 5% sodium carbonate solution. The organic phase was washed with brine, dried over MgSO₄ and concentrated in vacuo to give the crude product. Purification with flash column (methanol-DCM; gradient elution) yielded the Example 32 as white solid (25 mg, 6%). mp 143-144° C.; TLC single spot at R_{f}: 0.51 (35% EtOAc/DCM); ¹H NMR (270 MHz, CDCl₃) δ 1.61-1.70 (6H, m, 3×CH₂), 1.74 (6H, d, J=2.7 Hz, 3×CH₂), 2.05 (3H, broad, 3×CH), 4.33 (2H, s, CH₂), 7.79 (2H, dd, J=4.7, 1.4 Hz, ArH) and 8.90 (2H, dd, J=4.7, 1.4 Hz, ArH); LC/MS (ESI) m/z 320 (M⁺+H); tᵣ=1.00 min in 5% water-methanol; HRMS (ESI) calcd. for C₇H₂₂NO₃S (M⁺+H) 320.1320. found 320.1304; HPLC tᵣ=2.14 min (>99%) in 10% water-acetonitrile.

Example 33 was obtained as white solid (300 mg, 77%). mp 136-138° C.; TLC single spot at R_{f}: 0.46 (35% EtOAc/DCM); ¹H NMR (270 MHz, CDCl₃) δ 1.60-1.68 (6H, m, 3×CH₂), 1.74 (6H, d, J=2.7 Hz, 3×CH₂), 2.02 (3H, broad, 3×CH), 3.86 (1H, d, J=15.7 Hz, CH), 4.21 (1H, d, J=15.7 Hz, CH), 7.62 (2H, dd, J=4.7, 1.3 Hz, ArH) and 8.78 (2H, dd, J=4.7, 1.4 Hz, ArH); LC/MS (ESI) m/z 304 (M⁺+H); tᵣ=1.02 min in 5% water-methanol; HRMS (ESI) calcd. for C₁₇H₂₂NO₂S (M⁺+H) 304.1371. found 304.1359; HPLC tᵣ=2.02 min (>99%) in 10% water-acetonitrile.

### Example 34

### 6-{[2-(adamantan-1-yl)-2-oxoethyl]sulfanyl}-N-ethylpyridine-3-carboxamide

The title compound was synthesized with general amide formation method from 6-{[2-(adamantan-1-yl)-2-oxoethyl]sulfanyl}pyridine-3-carboxylic acid (80 mg, 0.24 mmol) and ethylamine (2M THF solution, 0.24 mL, 0.48 mmol). The title compound (70 mg, 81%) was obtained as white solid. mp 135.5-137° C.; TLC single spot at R_{f}: 0.66 (40% EtOAc/DCM); ¹H NMR (270 MHz, CDCl₃): δ 1.22 (3H, t, J=7.2 Hz, CH₃), 1.68-1.79 (6H, m, 3×CH₂), 1.93 (6H, d, J=2.7 Hz, 3×CH₂), 2.07 (3H, broad, 3×CH), 3.46 (2H, m, CH₂), 4.23 (2H, s, CH₂), 6.15 (1H, s, NH), 7.22 (1H, dd, J=8.6, 1.0 Hz, ArH), 7.80 (1H, dd, J=8.2, 2.2 Hz, ArH), and 8.66 (1H, d, J=1.6 Hz, ArH); LC/MS (ESI) m/z 359 (M⁺+H), tᵣ=1.19 min (99%) in 5% water-methanol; HRMS (ESI) calcd. for C₂₀H₂₇N₂O₂S (M⁺+H) 359.1793. found 359.1773; HPLC tᵣ=2.64 min (98%) in 10% water-acetonitrile.

### Example 35

### 6-(2-Adamantan-1-yl-2-oxo-ethylsulfanyl)-N,N-dimethyl-nicotinamide

The title compound was synthesized with general amide formation method from 6-{[2-(adamantan-1-yl)-2-oxoethyl]sulfanyl}pyridine-3-carboxylic acid (80 mg, 0.24 mmol) and dimethylamine (40% water solution, 0.06 mL, 0.48 mmol). The title compound (60 mg, 70%) was obtained as white solid. mp 62.5-64.5° C.; TLC single spot at R_{f}: 0.60 (30% EtOAc/DCM); ¹H NMR (270 MHz, CDCl₃): δ 1.65-1.79 (6H, m, 3×CH₂), 1.94 (6H, d, J=2.7 Hz, 3×CH₂), 2.07 (3H, broad, 3×CH), 3.02 (3H, s, CH₃), 3.07 (3H, s, CH₃), 4.23 (2H, s, CH₂), 7.24 (1H, dd, J=8.3, 0.8 Hz, ArH), 7.53 (1H, dd, J=8.3, 2.2 Hz, ArH), and 8.41 (1H, dd, J=2.2, 0.8 Hz, ArH); LC/MS (ESI) m/z 359 (M⁺+H), tᵣ=1.09 min (99%) in 5% water-methanol; HRMS (ESI) calcd. for C₂₀H₂₇N₂O₂S (M⁺+H) 359.1793. found 359.1778; HPLC tᵣ=2.61 min (99%) in 10% water-acetonitrile.

### Example 36

### 6-(2-Adamantan-1-yl-2-oxo-ethylsulfanyl)-N-methyl-nicotinamide

The title compound was synthesized with general amide formation method from 6-{[2-(adamantan-1-yl)-2-oxoethyl]sulfanyl}pyridine-3-carboxylic acid (80 mg, 0.24 mmol) and methylamine (40% water solution, 0.04 mL, 0.48 mmol). The title compound (38 mg, 46%) was obtained as white solid. mp 184-185.5° C.; TLC single spot at R_{f}: 0.57 (30% EtOAc/DCM); ¹H NMR (270 MHz, CDCl₃): δ 1.63-1.80 (6H, m, 3×CH₂), 1.94 (6H, d, J=2.7 Hz, 3×CH₂), 2.07 (3H, broad, 3×CH), 2.97 (3H, d, J=5.0 Hz, CH₃), 4.23 (2H, s, CH₂), 6.22 (1H, br, NH), 7.22 (1H, d, J=8.7 Hz, ArH), 7.80 (1H, dd, J=8.7, 1.6 Hz, ArH), and 8.66 (1H, d, J=1.6 Hz, ArH); LC/MS (ESI) m/z 345 (M⁺+H), tᵣ=1.11 min (99%) in 5% water-methanol; HRMS (ESI) calcd. for C₁₉H₂₅N₂O₂S (M⁺+H) 345.1637, 345.1623; HPLC tᵣ=2.40 min (99%) in 10% water-acetonitrile.

### Example 37

### 6-(2-Adamantan-1-yl-2-oxo-ethylsulfanyl)-nicotinamide

The title compound was synthesized with general amide formation method from 6-{[2-(adamantan-1-yl)-2-oxoethyl]sulfanyl}pyridine-3-carboxylic acid (130 mg, 0.39 mmol) and ammonia (7 N solution in methanol, 0.14 mL, 0.98 mmol). The title compound (38 mg, 30%) was obtained as white solid. mp 172-1735° C.; TLC single spot at R_{f}: 0.33 (50% EtOAc/DCM); ¹H NMR (270 MHz, CDCl₃): δ 1.69-1.82 (6H, m, 3×CH₂), 1.95 (6H, d, J=2.7 Hz, 3×CH₂), 2.08 (3H, br, 3×CH), 4.24 (2H, s, CH₂), 5.98 (2H, br, NH₂), 7.26 (1H, d, J=8.5 Hz, ArH), 7.86 (1H, dd, J=8.6, 2.5 Hz, ArH), and 8.72 (1H, d, J=2.2 Hz, ArH); LC/MS (ESI) m/z 331 (M⁺+H), tᵣ=1.05 min (99%) in 5% water-methanol; HRMS (ESI) calcd. for C₁₈H₂₃N₂O₂S (M⁺+H) 331.1480. found 331.1464; HPLC tᵣ=2.23 min (99%) in 10% water-acetonitrile.

### Example 38

### 1-Adamantan-1-yl-3-pyridin-3-yl-propenone

To a solution of adamantan-1-yl methyl ketone (256 mg, 2.0 mmol) in methanol (10 mL) was added pyridine-3-carbaldehyde (214 mg, 2.0 mmol), followed by NaOH (200 mg, 5.0 mmol). The mixture was stirred under nitrogen overnight, neutralized with 1N HCl and diluted with water. The solid was collected, washed with water and dried oin vacuo. Purification with flash column (DCM-ethyl acetate; gradient elution) yielded the title compound as yellow solid (330 mg, 62%). mp 107-108.5° C.; TLC single spot at R_{f}: 0.50 (30% EtOAc/DCM); ¹H NMR (270 MHz, CDCl₃) δ 1.69-1.80 (6H, m, 3×CH₂), 1.87 (6H, d, J=2.7 Hz, 3×CH₂), 2.08 (3H, br, 3×CH), 7.24 (1H, d, J=15.7 Hz, CH), 7.31 (1H, dd, J=8.0, 5.0 Hz, ArH), 7.62 (1H, d, J=15.7 Hz, CH), 7.86 (1H, dt, J=8.0, 1.9 Hz, ArH), 8.58 (1H, dd, J=4.6, 1.8 Hz, ArH) and 8.77 (1H, d, J=2.2 Hz, ArH); LC/MS (ESI) m/z 268 (M⁺+H); tᵣ=1.31 min (>99%) in 5% water-methanol; HRMS (ESI) calcd. for C₁₈H₂₂NO (M⁺+H) 268.1701. found 268.1699; HPLC tᵣ=3.17 min (>99%) in 10% water-acetonitrile.

### Example 39

### 1-Adamantan-1-yl-3-pyridin-3-yl-propan-1-one

The solution of 1-adamantan-1-yl-3-pyridin-3-yl-propenone (120 mg, 0.45 mmol) in methanol (20 mL) was hydrogenated over 10% Pd/C (75 mg) at atmosphere pressure for 12 h. After removing the catalyst by filtration through celite, the solution was concentrated to give the crude product. Purification with flash column (DCM-ethyl acetate; gradient elution) yielded the title compound as off-white solid (35 mg, 29%). mp 43-44.5° C.; TLC single spot at R_{f}: 0.55 (40% EtOAc/DCM); ¹H NMR (270 MHz, CDCl₃) δ 1.61-1.73 (6H, m, 3×CH₂), 1.74 (6H, d, J=2.8 Hz, 3×CH₂), 2.01 (3H, broad, 3×CH), 2.72-2.88 (4H, m, 2×CH₂), 7.18 (1H, dd, J=7.7, 4.9 Hz, ArH), 7.50 (1H, dd, J=7.7 Hz, ArH) and 8.41-8.43 (2H, m, ArH); LC/MS (ESI) m/z 270 (M⁺+H); tᵣ=1.23 min in 5% water-methanol; HRMS (ESI) calcd. for C₁₈H₂₄NO (M⁺+Na) 270.1858. found 270.1855; HPLC tᵣ=3.15 min (98%) in 10% water-acetonitrile.

### Example 40

### 1-[1-(4-Chloro-phenyl)-cyclohexyl]-2-(1-methyl-1H-imidazol-2-ylsulfanyl)-ethanone

2-Mercapto-1-methylimidazole (72 mg, 0.63 mmol) was added neat to a solution of 2-bromo-1-[1-(4-chloro-phenyl)-cyclohexyl]-ethanone (200 mg, 0.63 mmol) in CH₃CN (6 mL) at room temperature, then Et₃N (0.177 mL, 1.27 mmol) was added neat to the mixture and the reaction was stirred for 36 h. The traces of thiol left were quenched by addition of a small spatula of resin 2-chlorotrityl chloride, stirred for 1 h then the mixture was filtered and concentrated under vacuum. The crude mixture was purified by flash chromatography (hexane/EtOAc gradient 0-50%) to give the expected compound (20 mg, 95%) as transparent oil. TLC single spot at R_{f} 0.2 (hexane/EtOAc 5:5); ¹H NMR (270 MHz, CDCl₃): δ1.19-1.66 (m, 6H,), 1.73-1.87 (m, 2H), 2.23-2.36 (m, 2H), 3.56 (s, 3H), 3.90 (s, 2H), 6.82 (d, J=1.4 Hz, 1H), 6.91 (d, J=1.4 Hz, 1H), 7.13-7.20 (m, 2H), 7.22-7.29 (m, 2H); LC/MS (APCI) m/z 349 (M⁺); HPLC tᵣ=2.80 min (>99%) in 10% water-acetonitrile.

### Example 41

### 1-[1-(4-Chloro-phenyl)-cyclopentyl]-2-(1-methyl-1H-imidazol-2-ylsulfanyl)-ethanone

2-Mercapto-1-methylimidazole (57 mg, 0.50 mmol) was added neat to a solution of 2-bromo-1-[1-(4-chloro-phenyl)-cyclopentyl]-ethanone (150 mg, 0.50 mmol) in CH₃CN (5 mL) at room temperature, then Et₃N (0.139 mL, 1.00 mmol) was added neat to the mixture and the reaction was stirred overnight and over night. The traces of thiol left were quenched, by addition of a small spatula of resin 2-chlorotrityl chloride, stirred for 30 min. then the mixture was filtered and concentrated under vacuum. The crude mixture was purified by flash chromatography (hexane/EtOAc gradient 0-40%) to give the expected compound (20 mg, 90%) as yellow oil. TLC single spot at R_{f} 0.2 (hexane/EtOAc 6:4); ¹H NMR (270 MHz, CDCl₃): δ1.52-1.74 (m, 4H), 1.80-1.92 (m, 2H), 2.42-2.54 (m, 2H), 3.58 (s, 3H), 3.88 (s, 2H), 6.84 (d, J=1.1 Hz, 1H), 6.94 (d, J=1.0 Hz, 1H), 7.12-7.19 (m, 2H), 7.23-7.30 (m, 2H); LC/MS (APCI) m/z 335 (M⁺); HPLC tᵣ=2.60 min (>99%) in 10% water-acetonitrile.

### Example 42

### 1-[1-(4-Chloro-phenyl)-cyclobutyl]-2-(pyridin-2-ylmethoxy)-ethanone

1-[1-(4-Chloro-phenyl)-cyclobutyl]-2-hydroxy-ethanone (72 mg, 0.32 mmol) was dissolved in dry THF (3 mL) and cooled at 0° C. NaH was added in THF (1+1 mL) to the mixture and stir for 10 min. before 2-pycolylchloride HCl (57.4 mg, 0.35 mmol) was added neat and the reaction was slowly warmed up to room temperature over night. After 22 h the reaction was quenched by addition of water, extracted with ethyl acetate, and the organic phases were washed with brine and dried over MgSO₄. The crude reaction was purified by column chromatography on silica gel (hexanes/EtOAc 0-70% gradient) to afford the title compound (43 mg, 43%) as clear oil. TLC single spot at R_{f} 0.25 (hexane/EtOAc 7:3); ¹H NMR (270 MHz, CDCl₃): δ 1.82-2.08 (m, 2H), 2.38-2.52 (m, 2H), 2.71-2.83 (m, 2H), 4.66 (s, 4H), 7.22-7.26 (m, 1H), 7.46 (d, J=7.7 Hz, 1H), 7.71 (td, J=7.7, 1.9 Hz, 1H), 8.57 (d br, J=4.0 Hz, 1H); HPLC tᵣ=2.35 min (100%) in 20% water-methanol.

### Example 43

### 1-[1-(4-Chloro-phenyl)-cyclopentyl]-2-(1-methyl-1H-imidazole-2-sulfonyl)-ethanone

### Example 44

### 1-[1-(4-Chloro-phenyl)-cyclopentyl]-2-(1-methyl-1H-imidazole-2-sulfinyl)-ethanone

m-CPBA (132 mg, 0.59 mmol) was added neat to a solution of 1-[j-(4-chloro-phenyl)-cyclopentyl]-2-(1-methyl-1H-imidazol-2-ylsulfanyl)-ethanone (100 mg, 0.29 mmol) in dry DCM (5 mL) at 0° C. for 40 min. The reaction was quenched by addition of a saturated solution of NaHCO₃. The aqueous layer was extracted with DCM, the organic layers were washed with water then brine and dried over MgSO₄ before filtration and concentration under vacuum. The crude mixture was purified by flash chromatography (hexane/EtOAc gradient 0-80%) to give the expected sulfone Example 43 (11.6 mg, 11%) as off white solid and the expected sulfoxide Example 44 (66.9 mg, 66%) as transparent oil.

### Example 43

TLC single spot at R_{f} 0.51 (hexane/EtOAc 2:8); Mp=[138.4-141.3° C.]; ¹H NMR (270 MHz, CDCl₃): δ 1.52-1.76 (m, 4H), 1.76-1.92 (m, 2H), 2.34-2.48 (m, 2H), 3.99 (s, 3H), 6.98 (s br, 1H), 7.08-7.15 (m, 3H), 7.28-7.35 (m, 2H); LC/MS (APCI) m/z 365 (M⁺); HPLC tᵣ=2.35 min (100%) in 10% water-acetonitrile.

### Example 44

TLC single spot at R_{f} 0.12 (hexane/EtOAc 2:8); ¹H NMR (270 MHz, CDCl₃): δ1.46-1.76 (m, 4H), 1.76-1.96 (m, 2H), 2.42-2.58 (m, 2H), 3.87 (s, 3H), 4.20 (d, J=16.0 Hz, 1H), 4.66 (d, J=15.9 Hz, 1H), 6.95 (s br, 1H), 7.08 (d br, J=0.8 Hz, 1H), 7.15-7.21 (m, 2H), 7.28-7.34 (m, 2H); LC/MS (APCI) m/z 351 (M⁺, 40); HPLC tᵣ=2.21 min (100%) in 10% water-acetonitrile.

### Example 45

### 1-[1-(4-Chloro-phenyl)-cyclohexyl]-2-(1-methyl-H-imidazole-2-sulfonyl)-ethanone

### Example 46

### 1-[1-(4-Chloro-phenyl)-cyclohexyl]-2-(1-methyl-1H-imidazole-2-sulfinyl)-ethanone

m-CPBA (229 mg, 1.03 mmol) was added neat to a solution of 1-[1-(4-chloro-phenyl)-cyclohexyl]-2-(1-methyl-1H-imidazol-2-ylsulfanyl)-ethanone (179 mg, 0.51 mmol) in dry DCM (9 mL) at 0° C. for 2 hours. The reaction was quenched by addition of a saturated solution of NaHCO₃. The aqueous layer was extracted with DCM and then the organic layers were washed with water then brine and dried over MgSO₄ before filtration and concentration in vacuum. The crude mixture was purified by flash chromatography (hexane/EtOAc gradient 0-80%) to afford the expected sulfone Example 45 (84.1 mg, 43%) as off-white solid and the expected sulfoxide Example 46 (77.9 mg, 42%) as yellow oil.

### Example 45

TLC single spot at R_{f} 0.48 (hexane/EtOAc 3:7); Mp=[109.1-113.2° C.]; ¹H NMR (270 MHz, CDCl₃): δ 1.52-1.62 (m, 4H), 1.73-1.89 (m, 2H), 2.08-2.22 (m, 2H), 4.01 (s, 3H), 4.30 (s, 2H), 6.98 (s, 1H), 7.08-7.16 (m, 3H), 7.26-7.34 (m, 2H); LC/MS (APCI) m/z 403 (M⁺+Na); HPLC (01177a−1) tᵣ=2.44 min (98.8%) in 10% water-acetonitrile.

### Example 46

TLC single spot at R_{f} 0.10 (hexane/EtOAc 3:7); ¹H NMR (270 MHz, CDCl₃): δ1.20-1.66 (m, 6H), 1.73-1.92 (m, 2H), 2.15-2.32 (m, 2H), 3.59 (s, 3H), 4.19 (d, J=16.5 Hz, 1H), 4.67 (d, J=16.2 Hz, 1H), 6.94 (d, J=1.1 Hz, 1H), 7.06 (d, J=1.1 Hz, 1H), 7.14-7.21 (m, 2H), 7.26-7.33 (m, 2H); LC/MS (APCI) m/z 387.12 (M⁺+Na); HPLC tᵣ=2.29 min (98.9%) in 10% water-acetonitrile.

### Example 47

### 1-[1-(4-Chloro-phenyl)-cyclohexyl]-2-(pyridin-2-ylsulfanyl)-ethanone

2-Mercaptopyridine (66 mg, 0.59 mmol) was added neat to a solution of 2-bromo-1-[1-(4-chloro-phenyl)-cyclohexyl]-ethanone (186.9 mg, 0.59 mmol) in CH₃CN (6 mL) at room temperature, then Et₃N (0.165 mL, 1.18 mmol) was added neat to the mixture and the reaction was stirred overnight. The reaction was concentrated under vacuum. The crude mixture was purified by flash chromatography (hexane/EtOAc gradient 0-20%) to give the expected compound (158.7 mg, 77%) as yellow oil. TLC single spot at R_{f} 0.32 (hexane/EtOAc 8:2); ¹H NMR (270 MHz, CDCl₃): δ1.20-1.41 (m, 1H), 1.48-1.70 (m, 5H), 1.82-1.96 (m, 2H), 2.34-2.48 (m, 2H), 3.99 (s, 2H), 6.90 (dd br, J=7.4, 4.9 Hz, 1H), 7.11 (d br, J=8.3 Hz, 1H), 7.26-7.34 (m, 4H), 7.40 (td, J=7.4, 1.4 Hz, 1H), 8.16 (dt br, J=4.9 Hz, 1H); LC/MS (APCI) m/z 368 (M⁺+Na); Accurate mass (calculated MH⁺)=346.1027; (found)=346.1029; HPLC tᵣ=4.34 min (91.5%) in 10% water-acetonitrile.

### Example 48

### 1-[1-(4-Chloro-phenyl)-cyclohexyl]-2-(pyridine-2-sulfonyl)-ethanone

### Example 49

### 1-[1-(4-Chloro-phenyl)-cyclohexyl]-2-(pyridine-2-sulfinyl)-ethanone

m-CPBA (175 mg, 0.78 mmol) was added neat to a solution of 1-[1-(4-chloro-phenyl)-cyclohexyl]-2-(pyridin-2-ylsulfanyl)-ethanone (135 mg, 0.39 mmol) in dry DCM (7 mL) at 0° C. for 2 h30. The reaction was quenched by addition of a saturated solution of NaHCO₃. The aqueous layer was extracted with DCM then the organic layers were washed with water then brine and dried over MgSO₄ before filtration and concentration. The crude mixture was purified by flash chromatography (hexane/EtOAc gradient 0-40%) to give the expected sulfone Example 48 (114.9 mg, 78%) as white solid and the expected sulfoxide Example 49 (28.1 mg, 19%) as white solid.

### Example 48

TLC single spot at R_{f} 0.16 (hexane/EtOAc 7:3); Mp=[95.0-98.5° C.]; ¹H NMR (270 MHz, CDCl₃): δ 1.19-1.36 (m, 2H), 1.36-1.62 (m, 4H), 1.72-1.90 (m, 2H), 2.06-2.24 (m, 2H), 4.37 (s, 2H), 7.09-7.16 (m, 2H), 7.26-7.31 (m, 2H), 7.52 (ddd, J=7.4, 4.7, 1.1 Hz, 1H), 7.95 (td, J=7.7, 1.6 Hz, 1H), 8.05 (d br, J=7.7 Hz, 1H), 8.56 (d br, J=4.7 Hz, 1H); Accurate mass (calculated)=378.0925; (found)=378.0919; HPLC tᵣ=2.65 min (98.4%) in 10% water-acetonitrile.

### Example 49

TLC single spot at R_{f} 0.10 (hexane/EtOAc 3:7); Mp=[133.5-141.5° C.]; ¹H NMR (270 MHz, CDCl₃): δ1.15-1.40 (m, 1H), 1.40-1.70 (m, 5H), 1.75-1.95 (m, 2H), 2.17-2.36 (m, 2H), 3.75 (d, J=15.6 Hz, 1H), 3.99 (d, J=15.6 Hz, 1H), 7.14-7.37 (m, 5H), 7.83-7.94 (m, 2H), 8.50 (dt, J=4.7, 1.4 Hz, 1H); Accurate mass (calculated)=362.0976. (found)=362.0982; HPLC tᵣ=2.63 min (100%) in 10% water-acetonitrile.

### Example 50

### 1-[1-(4-Chloro-phenyl)-cyclopentyl]-2-(6-methyl-pyridin-3-yloxy)-ethanone

5-Hydroxy-2-methylpyridine (14 mg, 0.12 mmol) then K₂CO₃ (33 mg, 0.24 mmol) were added to a solution of 2-bromo-1-[1-(4-chloro-phenyl)-cyclopentyl]-ethanone (37.4 mg, 0.12 mmol) in acetone (3 mL) at room temperature. The reaction was stirred 21 h at room temperature then was quenched by addition of water. The extraction was conducted with EtOAc (×2) then the organic phase was washed with brine and dried over MgSO₄. The crude residue was the purified by flash chromatography (hex/EtOAc 0-40% gradient) to give the expected title compound (33.5 mg, 84%) as clear oil. TLC single spot at R_{f} 0.11 (hexane/EtOAc 6:4); ¹H NMR (270 MHz, CDCl₃): δ1.90-2.02 (m, 2H), 2.43 (s, 3H), 2.45-2.60 (m, 2H), 4.54 (s, 2H), 6.76 (dd, J=8.5, 3.0 Hz, 1H), 6.93 (d, J=8.5 Hz, 1H), 7.20-7.27 (m, 2H), 7.28-7.35 (m, 2H), 7.95 (d, J=3.0 Hz, 1H); LC/MS (APCI) m/z 330 (M⁺); HPLC tᵣ=2.85 min (100%) in 10% water-acetonitrile.

### Example 51

### 1-[1-(4-Chloro-phenyl)-cyclohexyl]-2-(6-methyl-pyridin-3-yloxy)-ethanone

5-Hydroxy-2-methylpyridine (43 mg, 0.39 mmol) then K₂CO₃ (108 mg, 0.78 mmol) were added to a solution of 2-bromo-1-[1-(4-chloro-phenyl)-cyclohexyl]-ethanone (124.2 mg, 0.39 mmol) in acetone (5 mL) at room temperature. The reaction was stirred over night (20 h) at room temperature then was quenched by addition of water. The extraction was conducted with EtOAc (×2) then the organic phase was washed with brine and dried over MgSO₄. The crude residue was the purified by flash chromatography (hex/EtOAc 0-40% gradient) to afford the title compound (120 mg, 89%) as clear oil. TLC single spot at R_{f} 0.15 (hexane/EtOAc 6:4); ¹H NMR (270 MHz, CDCl₃): δ 1.26-1.70 (m, 2H), 1.78-1.94 (m, 2H), 2.28-2.42 (m, 2H), 2.42 (s, 3H), 4.56 (s, 2H), 6.68 (dd, J=8.5, 3.0 Hz, 1H), 6.91 (d, J=8.5 Hz, 1H), 7.22-7.36 (m, 4H), 7.95 (d, J=3.0 Hz, 1H); LC/MS (APCI) m/z 344 (M⁺); HPLC tᵣ=3.22 min (100%) in 10% water-acetonitrile.

### Example 52

### 3-(4-Chloro-phenyl)-3-methyl-1-(1-methyl-1H-imidazol-2-ylsulfanyl)-butan-2-one

2-Mercapto-1-methylimidazol (40.8 mg, 0.35 mmol) was neat to a solution of 1-bromo-3-(4-chloro-phenyl)-3-methyl-butan-2-one (98.5 mg, 0.35 mmol) in CH₃CN (5 mL) at room temperature, then Et₃N (0.098 mL, 0.70 mmol) was added neat to the mixture and the reaction was stirred overnight. Thiol residues were trapped by addition of a small spatula of resin 2-chlorotrityl chloride in 30 min then the mixture was filtered and concentrated under vacuum. The crude mixture was purified by flash chromatography (hexane/EtOAc gradient 0-40%) to give the expected compound (108.5 mg, 100%) as brown solid. TLC single spot at R_{f} 0.47 (MeOH/DCM 1:9); Mp=[43.7-47.3° C.]; ¹H NMR (270 MHz, CDCl₃): δ 1.46 (s, 6H), 3.59 (s, 3H), 3.86 (s, 2H), 6.84 (d, J=1.1 Hz, 1H), 6.94 (d, J=1.4 Hz, 1H), 7.07-7.15 (m, 2H), 7.23-7.30 (m, 2H); Accurate Mass: calculated (M⁺+H) 309.0823. found 309.0811; HPLC tᵣ=2.06 min (100%) in 10% water-acetonitrile.

### Example 53

### 1-[1-(4-Chloro-phenyl)-cyclobutyl]-2-(6-methyl-pyridin-3-yloxy)-ethanone

5-Hydroxy-2-methylpyridine (39 mg, 0.36 mmol) then K₂CO₃ (99.5 mg, 0.72 mmol) were added to a solution of 2-bromo-1-[1-(4-chloro-phenyl)-cyclobutyl]-ethanone (104 mg, 0.36 mmol) in acetone (5 mL) at room temperature. The reaction was stirred 24 h at room temperature then was quenched by addition of water. The extraction was conducted with EtOAc (×2) then the organic phase was washed with brine and dried over MgSO₄. The crude residue was the purified by flash chromatography (hexane/EtOAc 0-50% gradient) to afford the title compound (116.2 mg, >99%) as orange solid. TLC single spot at R_{f} 0.13 (hexane/EtOAc 6:4); Mp=[48.5-50.3° C.]; ¹H NMR (270 MHz, CDCl₃): δ1.84-2.05 (m, 2H), 2.44 (s, 3H), 2.38-2.51 (m, 2H), 2.77-2.90 (m, 2H), 4.50 (s, 2H), 6.83 (dd, J=8.5, 3.0 Hz, 1H), 6.96 (d, J=8.3 Hz, 1H), 7.16-7.24 (m, 2H), 7.29-7.37 (m, 2H), 7.98 (1H, d, J=3.0 Hz); LC/MS (APCI) m/z 316 (M⁺); HPLC tᵣ=2.29 min (>99%) in 10% water-acetonitrile.

### Example 54

### 1-[1-(4-Chloro-phenyl)-cyclopropyl]-2-(6-methyl-pyridin-3-yloxy)-ethanone

5-Hydroxy-2-methylpyridine (60 mg, 0.55 mmol) then K₂CO₃ (152 mg, 1.10 mmol) were added to a solution of 2-bromo-1-[1-(4-chloro-phenyl)-cyclopropyl]-ethanone (150 mg, 0.55 mmol, impure compound) in acetone (7 mL) at room temperature. The reaction was stirred 20 h at room temperature then was quenched by addition of water. The extraction was conducted with EtOAc (×2) then the organic phase was washed with brine and dried over MgSO₄. The crude residue was the purified by flash chromatography (hexane/EtOAc 0-50% gradient) to afford the title compound (29 mg, 17%) as orange solid. TLC single spot at R_{f} 0.14 (hexane/EtOAc 6:4); Mp=[75.3-81.4° C.]; ¹H NMR (270 MHz, CDCl₃): δ 1.19-1.29 (m, 2H), 1.69-1.75 (m, 2H), 2.44 (s, 3H), 4.55 (s, 2H), 6.94 (dd, J=8.5, 3.0 Hz, 1H), 7.00 (d, J=8.5 Hz, 1H), 7.36 (s, 4H), 7.99 (d, J=2.7 Hz, 1H); LC/MS (APCI) m/z 302 (M⁺); HPLC tᵣ=2.03 min (97%) in 10% water-acetonitrile.

### Example 55

### 1-Adamantan-1-yl-2-(5-trifluoromethyl-pyridin-2-yloxy)-ethanone

K₂CO₃ (107 mg, 0.78 mmol) was added to a solution of 1-adamantyl bromomethyl ketone (100 mg, 0.39 mmol) and 5-tri(fluoromethyl)-2-pyridinol (64 mg, 0.39 mmol) in acetone (5 mL) at room temperature. The reaction was stirred for 20 h at room temperature then was quenched by addition of water. The extraction was conducted with EtOAc (×2) then the organic phase was washed with brine and dried over MgSO₄. The crude residue was the purified by flash chromatography (hexane/EtOAc 0-20% gradient) to afford the title compound (118.6 mg, 90%) as white solid. TLC single spot at R_{f} 0.22 (hexane/EtOAc 7:3); Mp=[101.7-103.5° C.]; ¹H NMR (270 MHz, CDCl₃): δ2.65-2.83 (m, 2H), 1.93 (d, J=2.7 Hz, 6H), 2.08 (br s, 3H), 4.85 (s, 2H), 6.58 (d br, J=10.4 Hz, 1H), 7.48-7.40 (m, 2H); LC/MS (APCI) m/z 338 (M⁺−H), 339 (M⁺); Accurate Mass: calculated (M⁺+H) 340.1519. found 340.1521; HPLC tᵣ=2.20 min (100%) in 10% water-acetonitrile.

### Example 56

### 1-Adamantan-1-yl-2-(6-trifluoromethyl-pyridin-3-ylmethoxy)-ethanone

NaH (23 mg, 1.56 mmol) was added neat at 0° C. to a solution of 6-(trifluoromethyl)pyridin-3-methanol (0.098 mL, 0.78 mmol) in dry THF (5 mL) and stirred 30 min. Then 1-adamantyl bromomethyl ketone (200 mg, 0.78 mmol) was added via a cannula to the suspension and the reaction was stirred for 24 h. The reaction was quenched by addition of water. The extraction was conducted with EtOAc (×2) then the organic phase was washed with brine and dried over MgSO₄. The crude residue was the purified by flash chromatography (hexane/EtOAc 0-20% gradient) to afford the title compound (76.6 mg, 28%) as white solid. TLC single spot at R_{f} 0.31 (hexane/EtOAc 7:3); Mp=[105.7-109.8° C.]; ¹H NMR (270 MHz, CDCl₃): δ1.58-1.80 (m, 6H), 1.80 (d, J=2.7 Hz, 6H), 2.01 (s br, 3H), 4.38 (s, 2H), 4.63 (s, 2H), 7.65 (d, J=7.9 Hz, 1H), 7.93 (dd, J=7.9, 1.4 Hz, 1H), 8.65 (s br, 1H); LC/MS (APCI) m/z 376 (M⁺+Na); Accurate Mass: calculated (M⁺+H) 354.1675. found 354.1664; HPLC tᵣ=3.22 min (96.6%) in 5% water-methanol.

### Example 57

### 1-Adamantan-1-yl-2-(5-chloro-pyridin-3-yloxy)-ethanone

K₂CO₃ (108 mg, 0.78 mmol) was added to a solution of 1-adamantyl bromomethyl ketone (100 mg, 0.39 mmol) and 5-chloro-3-pyridinol (50 mg, 0.39 mmol) in acetone (5 mL) at room temperature. The reaction was stirred for 20 h then was quenched by addition of water. The extraction was conducted with EtOAc (×2) then the organic phase was washed with brine and dried over MgSO₄. The crude residue was the purified by flash chromatography (hexane/EtOAc 0-30% gradient) to afford the title compound (89.9 mg, 75.5%) as pearl white solid. TLC single spot at R_{f} 0.34 (hexane/EtOAc 7:3); Mp=[126.5-128.0° C.]; ¹H NMR (270 MHz, CDCl₃): δ1.63-1.82 (m, 6H), 1.88 (d, J=2.5 Hz, 6H), 2.07 (s br, 3H), 4.89 (s, 2H), 7.06-7.12 (m, 1H), 8.12 (d, J=2.2 Hz, 1H), 8.17 (d, J=1.4 Hz, 1H); LC/MS (APCI) m/z 328 (M⁺+Na); Accurate Mass: calculated (M⁺+H) 306.1255. found 306.1244; HPLC tᵣ=2.82 min (100%) in 10% water-acetonitrile.

### Example 58

### 1-Adamantan-1-yl-2-(6-chloro-pyridin-2-yloxy)-ethanone

K₂CO₃ (108 mg, 0.78 mmol) was added to a solution of 1-adamantyl bromomethyl ketone (100 mg, 0.39 mmol) and 6-chloro-2-pyridinol (50.5 mg, 0.39 mmol) in acetone (5 mL) at room temperature. The reaction was stirred for 20 h at room temperature then was quenched by addition of water. The extraction was conducted with EtOAc (×2) then the organic phase was washed with brine and dried over MgSO₄. The crude residue did not require any purification and the expected ether (119 mg, >99%) was isolated as white solid. TLC single spot at R_{f} 0.40 (hexane/EtOAc 7:3); Mp=[89.5-93.2° C.]; ¹H NMR (270 MHz, CDCl₃): δ1.65-1.82 (m, 6H), 1.93 (d, J=2.5 Hz, 6H), 2.06 (s br, 3H), 5.10 (s, 2H), 6.76 (d, J=8.3 Hz, 1H), 6.85 (d, J=7.5 Hz, 1H), 7.51 (t, J=8.3 Hz, 1H); LC/MS (APCI) m/z 328 (M⁺+Na); Accurate Mass: calculated (M⁺+H) 306.1255. found 306.1261; HPLC tᵣ=4.04 min (100%) in 10% water-acetonitrile.

### Example 59

### 1-Adamantan-1-yl-2-(6-trifluoromethyl-pyridin-3-ylmethylsulfanyl)-ethanone

NaOH (1M in water, 1.08 mL, 1.08 mmol) was added to a solution of thioacetic acid S-(2-adamantan-1-yl-2-oxo-ethyl) ester (272 mg, 1.08 mmol) in acetone (5 mL) at room temperature (T=16° C.). When the staring material was consumed, a solution of 5-chloromethyl-2-trifluoromethyl-pyridine (211 mg, 1.08 mmol) and Et₃N (150 μL, 1.08 mmol) in CH₃CN (5 mL) was added via a cannula and stirred over night. The reaction was quenched with water, extracted with EtOAc (×2) then the organic phase was washed with brine and dried over MgSO₄. The crude was purified by flash chromatography (hexane/EtOAc 0-10% gradient) to give the expected compound (78.6 mg, 20%) as yellow solid. TLC single spot at R_{f} 0.50 (hexane/EtOAc 7:3); Mp=[79.7-82.2° C.]; ¹H NMR (270 MHz, CDCl₃): δ1.58-1.80 (m, 6H), 1.80 (d, J=2.7 Hz, 6H), 2.02 (s br, 3H), 3.18 (s, 2H), 3.76 (s, 2H), 7.62 (d, J=8.0 Hz, 1H), 7.88 (dd, J=8.0, 1.4 Hz, 1H), 8.65 (s br, 1H); LC/MS (APCI) m/z 368 (M⁺−1H); Accurate Mass: calculated (M+H)⁺ 370.1447. found 370.1436; HPLC tᵣ=3.44 min (97.5%) in 10% water-acetonitrile.

### Example 60

### 1-Adamantan-1-yl-2-(6-chloro-pyridin-2-yloxy)-ethanone

K₂CO₃ (108 mg, 0.78 mmol) was added to a solution of 1-adamantyl bromomethyl ketone (100 mg, 0.39 mmol) and 2-chloro-3-pyridinol (50.5 mg, 0.39 mmol) in acetone (5 mL) at room temperature. The reaction was stirred for 20 h at room temperature then was quenched by addition of water. The extraction was conducted with EtOAc (×2) then the organic phase was washed with brine and dried over MgSO₄. The crude residue did not require any purification and the expected compound (117.4 mg, 98%) was isolated as off-white solid. TLC single spot at R_{f} 0.26 (hexane/EtOAc 7:3); Mp=[124.2-128.9° C.]; ¹H NMR (270 MHz, CDCl₃): δ1.63-1.82 (m, 6H), 1.88 (d, J=2.8 Hz, 6H), 2.05 (s br, 3H), 4.94 (s, 2H), 6.92 (dd, J=8.0, 1.4 Hz, 1H), 7.10 (dd, J=8.0, 4.7 Hz, 1H), 7.96 (dd, J=4.7, 1.4 Hz, 1H); LC/MS (APCI) m/z 328.04 (M⁺+Na); Accurate Mass: calculated (M⁺+H) 306.1255. found 306.1246; HPLC tᵣ=2.38 min (99%) in 10% water-acetonitrile.

### Example 61

### 1-Adamantan-1-yl-2-(pyridin-3-yloxy)-ethanone

To a solution of 3-hydroxypyridine (95 mg, 1.0 mmol) in methanol (2 mL) was added CH₃ONa (60 mg, 1.1 mmol). The mixture was stirred under nitrogen at rt for 30 min, evaporated to dryness and added to a solution of adamantan-1-yl bromomethyl ketone (257 mg, 1.0 mmol) in DMF (3 mL). The mixture was stirred under nitrogen overnight, diluted with water and extracted with DCM. The organic phase was washed with brine, dried over MgSO₄ and concentrated in vacuo to give the crude product. Purification with flash column (DCM-ethyl acetate; gradient elution) yielded the title compound as off-white solid (50 mg, 18%). mp 73-75° C.; TLC single spot at R_{f}: 0.57 (50% EtOAc/DCM); ¹H NMR (270 MHz, CDCl₃): δ 1.75-1.82 (6H, m, 3×CH₂), 1.92 (6H, d, J=2.7 Hz, 3×CH₂), 2.08 (3H, br, 3×CH), 4.91 (2H, s, CH₂), 7.10-7.21 (2H, m, ArH), 8.22 (1H, dd, J=4.6, 1.3 Hz, ArH), and 8.26 (1H, d, J=2.7 Hz, ArH); LC/MS (ESI) m/z 270 (M⁺−H), tᵣ=1.09 min (99%) in 5% water-methanol; HRMS (ESI) calcd. for C₁₇H₂₂NO₂ (M⁺+H) 272.1651. found 272.1672; HPLC tᵣ=2.39 min (96%) in 10% water-acetonitrile.

### Example 62

### 1-Adamantan-1-yl-2-(6-chloro-pyridin-3-ylmethylsulfanyl)-ethanone

To a solution of 1-(adamantan-1-yl)-2-(acetylsulfanyl)ethan-1-one (504 mg, 2.0 mmol) in acetone (4 mL) was added 1N NaOH (2.0 mL, 2.0 mmol)). The mixture was stirred at rt under nitrogen for 1 h, added to a solution of 2-chloro-5-(chloromethyl)pyridine (324 mg, 2.0 mmol) in CH₃CN-Et₃N (4 mL-2 mL). The mixture was stirred at rt for 24 h, partitioned between EtOAc and water. The organic phase was washed brine, dried over MgSO₄ and concentrated in vacuo. Purification with flash column (EtOAc-hexane gradient elution) gave product (220 mg, 33%) as off-white solid. mp 88-90° C.; TLC single spot at R_{f}: 0.66 (30% EtOAc/hexane); ¹H NMR (270 MHz, CDCl₃) δ 1.63-1.81 (6H, m, 3×CH₂), 2.03 (6H, d, J=2.7 Hz, 3×CH₂), 2.04 (3H, br, 3×CH), 3.19 (2H, s, CH₂), 3.68 (2H, s, CH₂), 7.26 (1H, d, J=8.3 Hz, ArH), 7.68 (1H, dd, J=8.3, 2.5 Hz, ArH) and 8.31 (1H, d, J=2.5 Hz, ArH); LC/MS (ESI) m/z 334 (M⁺−H), tᵣ=1.30 min in 5% water-methanol; HRMS (ESI) calcd. for C₁₈H₂₃ClNOS (M⁺+H) 336.1189. found 336.1171; HPLC tᵣ=4.10 min (95%) in 30% water-acetonitrile.

### Example 63

### 1-Adamantan-1-yl-2-(4-methyl-4H-[1,2,4]triazole-3-sulfinyl)-ethanone

To a cold solution of 1-Adamantan-1-yl-2-(4-methyl-4H-[1,2,4]triazol-3-ylsulfanyl)-ethanone (200 mg, 0.69 mmol) in DCM (20 mL) was added mCPBA (188 mg, purity 60-77%). The mixture was stirred at −10° C. for 50 min, partitioned between DCM and 5% sodium carbonate solution. The organic phase was washed with brine, dried over MgSO₄ and concentrated in vacuo. Purification with flash column (methanol-DCM; gradient elution) yielded the title compound as white solid (180 mg, 85%). mp 124.5-127° C.; TLC single spot at R_{f}: 0.49 (10% CH₃OH/DCM); ¹H NMR (270 MHz, CDCl₃) δ 1.62-1.84 (12H, m, 6×CH₂), 2.07 (3H, br, 3×CH), 3.97 (3H, s, CH₃), 4.62 (1H, d, J=15 Hz, CH), 5.09 (1H, d, J=15 Hz, CH) and 8.20 (1H, s, ArH); LC/MS (ESI) m/z 306 (M⁺−H); tᵣ=0.93 min in 5% water-methanol; HRMS (ESI) calcd. for C₁₅H₂₁N₃O₂SNa (M⁺+Na) 330.1252. found 330.1223; HPLC tᵣ=2.10 min (94%) in 10% water-acetonitrile.

### Example 64

### 1-Adamantan-1-yl-2-(6-chloro-pyridin-3-ylmethanesulfonyl)-ethanone and Example 65: 1-Adamantan-1-yl-2-(6-chloro-pyridin-3-ylmethanesulfinyl)-ethanone

To a cold solution of 1-adamantan-1-yl-2-(6-chloro-pyridin-3-ylmethylsulfanyl)-ethanone (180 mg, 0.54 mmol) in DCM (20 mL) was added mCPBA (147 mg, purity 60-77%).

The mixture was stirred at −10° C. for 45 min, partitioned between DCM and 5% sodium carbonate solution. The organic phase was washed with brine, dried over MgSO₄ and concentrated in vacuo. Purification with flash column (EtOAc-DCM; gradient elution) yielded Example 64 as white solid (28 mg, 14%). mp 159-161° C.; TLC single spot at R_{f}: 0.69 (20% EtOAc/DCM); ¹H NMR (270 MHz, CDCl₃) δ 1.62-1.78 (6H, m, 3×CH₂), 1.79 (6H, d, J=2.7 Hz, 3×CH₂), 2.08 (3H, br, 3×CH), 3.90 (2H, s, CH₂), 4.52 (2H, s, CH₂), 7.38 (1H, d, J=8.3 Hz, ArH), 7.82 (1H, dd, J=8.3, 2.5 Hz, ArH) and 8.47 (1H, d, J=2.5 Hz, ArH); LC/MS (ESI) m/z 366 (M⁺−H); tᵣ=1.02 min in 5% water-methanol; HRMS (ESI) calcd. for C₁₈H₂₂ClNO₃SNa (M⁺+Na) 390.0907. found 390.0886; HPLC tᵣ=1.02 min (96%) in 10% water-acetonitrile.

Example 65 was obtained as white solid (92 mg, 48%). mp 153-154° C.; TLC single spot at R_{f}: 0.27 (20% EtOAc/DCM); ¹H NMR (270 MHz, CDCl₃) δ 1.63-1.77 (12H, m, 6×CH₂), 2.07 (3H, br, 3×CH), 3.55 (1H, d, J=16 Hz, CH), 3.96 (1H, d, J=16 Hz, CH), 4.00 (1H, d, J=14 Hz, CH), 4.24 (1H, d, J=14 Hz, CH), 7.37 (1H, d, J=8.2 Hz, ArH), 7.68 (1H, dd, J=8.2, 2.5 Hz, ArH) and 8.27 (1H, d, J=2.4 Hz, ArH); LC/MS (ESI) m/z 350 (M⁺−H); tᵣ=1.00 min in 5% water-methanol; HRMS (ESI) calcd. for C₁₈H₂₂ClNO₂SNa (M⁺+Na) 374.0957. found 374.0946; HPLC tᵣ=1.45 min (>99%) in 10% water-acetonitrile.

### Example 66

### 1-Adamantan-1-yl-2-(5-trifluoromethyl-pyridin-2-ylsulfanyl)-ethanone

To a solution of adamantan-1-yl bromomethyl ketone (514 mg, 2.0 mmol) in acetonitrile (15 mL) was added 6-(trifluoromethyl)pyridine-2-thiol (360 mg, 2.2 mmol), followed by triethylamine (0.5 mL). The mixture was stirred at ambient temperature overnight, partitioned between ethyl acetate and saturated sodium bicarbonate. The organic phase was washed with brine, dried over MgSO₄ and concentrated in vacuo to give the title compound as white solid (610 mg, 86%). mp 141-142° C.; TLC single spot at R_{f}: 0.72 (20% EtOAc/hexane); ¹H NMR (270 MHz, CDCl₃) δ 1.69-1.80 (6H, m, 3×CH₂), 1.94 (6H, d, J=2.7 Hz, 3×CH₂), 2.08 (3H, br, 3×CH), 4.26 (2H, s, CH₂), 7.30 (1H, t, J=8.6 Hz, ArH), 7.64 (1H, dd, J=8.5, 2.5 Hz, ArH) and 8.55 (1H, s, ArH); LC/MS (ESI) m/z 354 (M⁺−H); tᵣ=1.50 min in 5% water-methanol; HRMS (ESI) calcd. for C₁₈H₂₀F₃NOSNa (M⁺+Na) 378.1115. found 378.109; HPLC tᵣ=6.63 min (>99%) in 10% water-acetonitrile.

### Example 67

### 1-Adamantan-1-yl-2-(5-trifluoromethyl-pyridine-2-sulfonyl)-ethanone and Example 68: 1-Adamantan-1-yl-2-(5-trifluoromethyl-pyridine-2-sulfinyl)-ethanone

To a cold (−5° C.) solution of 1-adamantan-1-yl-2-(5-trifluoromethyl-pyridin-2-ylsulfanyl)-ethanone (470 mg, 1.32 mmol) in DCM (40 mL) was added mCPBA (362 mg, purity 60-77%). The mixture was stirred at −5° C. for 45 min, partitioned between DCM and 5% sodium carbonate solution. The organic phase was washed with brine, dried over MgSO₄ and concentrated in vacuo. Purification with flash column (EtOAc-hexane; gradient elution) yielded the Example 67 as white solid (157 mg, 31%). mp 138-139° C.; TLC single spot at R_{f}: 0.42 (40% EtOAc/hexane); ¹H NMR (270 MHz, CDCl₃) δ 1.54-1.77 (12H, m, 6×CH₂), 2.06 (3H, br, 3×CH), 4.72 (2H, s, CH₂), 8.25 (2H, s, ArH) and 8.93 (1H, s, ArH); LC/MS (ESI) m/z 386 (M⁺−H); tᵣ=2.59 min in 20% water-methanol; HRMS (ESI) calcd. for C₁₈H₂₀F₃NO₃SNa (M⁺+Na) 410.1014. found 410.0985; HPLC tᵣ=2.36 min (97%) in 10% water-acetonitrile.

Example 68 was obtained as white solid (118 mg, 24%). TLC single spot at R_{f}: 0.36 (40% EtOAc/hexane); ¹H NMR (270 MHz, CDCl₃) δ 1.67-1.81 (12H, m, 6×CH₂), 2.05 (3H, br, 3×CH), 4.08 (1H, d, J=15 Hz, CH), 4.35 (1H, d, J=15 Hz, CH), 4.38 (2H, s, CH₂), 8.18 (2H, s, ArH) and 8.86 (1H, s, ArH); LC/MS (ESI) m/z 370 (M⁺−H); tᵣ=2.92 min in 20% water-methanol; HRMS (ESI) calcd. for C₁₈H₂₀F₃NO₂SNa (M⁺+Na) 394.1065. found 394.1042; HPLC tᵣ=2.41 min (98%) in 10% water-acetonitrile.

### Example 69

### 1-Adamantan-1-yl-2-(6-trifluoromethyl-pyridin-3-ylmethanesulfonyl)-ethanone

m-CPBA (76 mg, 0.33 mmol) was added neat to a solution of 1-adamantan-1-yl-2-(6-trifluoromethyl-pyridin-3-ylmethylsulfanyl)-ethanone (60.8 mg, 0.16 mmol) in dry DCM (4 mL) at 0° C. for 1 hour. The reaction was quenched by addition of a saturated solution of NaHCO₃, extracted with DCM, washed with water then brine, dried over MgSO₄ before filtration and concentration under reduced pressure. The crude mixture was purified by flash chromatography on silica gel using a gradient of 0-40% EtOAc in hexane to give the expected sulfone (18.3 mg, 28%) as white solid. TLC single spot at R_{f} 0.14 (hexane/EtOAc 8:2); Mp=[145.3-149.1° C.]; ¹H NMR (270 MHz, CDCl₃): δ 1.60-1.76 (m, 6H), 1.79 (d, J=2.7 Hz, 6H), 2.08 (br s, 3H), 3.93 (s, 2H), 4.07 (s, 2H), 7.72 (d, J=8.2 Hz, 1H), 8.04 (dd, J=7.7, 1.7 Hz, 1H), 8.80 (s, 1H); LC/MS (APCI) m/z 400 (M⁺−H); HPLC tᵣ=2.12 min (97%) in 10% water-methanol.

### Example 70

### 2-(6-Methyl-pyridin-3-yloxy)-1-(2,4,6-trimethyl-phenyl)-ethanone

K₂CO₃ (113 mg, 0.82 mmol) was added to a solution of 2-bromo-1-(2,4,6-trimethyl-phenyl)-ethanone (100 mg, 0.41 mmol) and 5-hydroxy-2-methylpyridine (45 mg, 0.41 mmol) in acetone (5 mL) at room temperature. The reaction was stirred for 20 h at room temperature then was quenched by addition of water. The extraction was conducted with EtOAc (×2) then the organic phase was washed with brine and dried over MgSO₄. The crude residue was the purified by flash chromatography (DCM/MeOH 0-5% gradient) to give the expected compound (62.7 mg, 57%) as yellow oil. TLC single spot at R_{f} 0.46 (DCM/MeOH 9:1); ¹H NMR (270 MHz, CDCl₃): δ2.22 (6H, s), 2.27 (3H, s), 2.46 (3H, s), 4.86 (2H, s), 6.84 (2H, s), 7.02 (1H, d, J=8.3 Hz), 7.11 (1H, dd, J=2.8, 8.5 Hz), 8.16 (1H, d, J=2.8 Hz); LC/MS (APCI) m/z 270 (M⁺+H); HPLC tᵣ=1.84 min (100%) in 10% water-acetonitrile.

### Example 71

### 3-(4-Chloro-phenyl)-3-methyl-1-(6-methyl-pyridin-3-yloxy)-butan-2-one

5-Hydroxy-2-methylpyridine (46 mg, 0.42 mmol) and K₂CO₃ (116 mg, 0.84 mmol) were added to a solution of 1-bromo-3-(4-chloro-phenyl)-3-methyl-butan-2-one (116 mg, 0.42 mmol) in acetone (5 mL) at room temperature. The reaction was stirred overnight at RT then quenched with water. The extraction was done with EtOAc (2×), then the organic layer was washed with brine and dried over MgSO₄. The crude residue was the purified by flash chromatography (hexane/EtOAc 0-40% gradient) to afford the expected compound (67 mg, 53%) as off white solid. TLC single spot at R_{f} 0.9 (hexane/EtOAc 7:3); Mp=[68.5-70.9° C.]; ¹H NMR (270 MHz, CDCl₃): δ1.54 (s, 6H), 2.42 (s, 3H), 4.55 (s, 2H), 6.82 (dd, J=3.0, 8.5 Hz, 1H), 6.94 (d, J=8.5 Hz, 1H), 7.18-7.26 (m, 2H), 7.27-7.36 (m, 2H), 7.96 (d, J=3.0 Hz, 1H); LC/MS (APCI) m/z 304 (M⁺); HPLC tᵣ=1.98 min (99%) in 10% water-acetonitrile.

### Example 72

### 2-(Pyridin-3-yloxy)-1-(2,4,6-trimethyl-phenyl)-ethanone

K₂CO₃ (113 mg, 0.82 mmol) was added to a solution of 2-bromo-1-(2,4,6-trimethyl-phenyl)-ethanone (100 mg, 0.41 mmol) and 3-hydroxypyridine (39 mg, 0.41 mmol) in acetone (5 mL) at room temperature. The reaction was stirred overnight at room temperature then was quenched with of water. The extraction was conducted with EtOAc (×2) then the organic phase was washed with brine and dried over MgSO₄. The crude residue was the purified by flash chromatography (hexane/EtOAc 0-40% gradient) to give the expected compound (17.3 mg, 17%) as brown oil. TLC single spot at R_{f} 0.9 (hexane/EtOAc 7:3); ¹H NMR (270 MHz, CDCl₃): δ2.24 (s, 6H), 2.28 (s, 3H), 4.90 (s, 2H), 6.86 (s, 2H), 7.21 (t, J=2.7 Hz, 2H), 8.25 (t, J=3.0 Hz, 1H), 8.31 (s br, 1H); LC/MS (APCI) m/z 256 (M⁺+H); HPLC tᵣ=1.85 min (98.8%) in 10% water-acetonitrile.

### Example 73

### 2-(Pyridin-2-ylsulfanyl)-1-(2,4,6-trimethyl-phenyl)-ethanone

Et₃N (0.136 mL, 0.98 mmol) was added to a solution of 2-bromo-1-(2,4,6-trimethyl-phenyl)-ethanone (119 mg, 0.49 mmol) and 2-mercaptopyridine (54 mg, 0.49 mmol) in CH₃CN (5 mL) at room temperature. The reaction was stirred overnight at room temperature then was quenched with of a saturated solution of NH₄Cl. The extraction was conducted with DCM (×2) then the organic phase was washed with brine and dried over MgSO₄. The crude residue was the purified by flash chromatography (hexane/EtOAc 0-30% gradient) to give the expected compound (137 mg, >99%) as yellow oil. TLC single spot at R_{f} 0.6 (hexane/EtOAc 6:4); ¹H NMR (270 MHz, CDCl₃): δ2.26 (s, 9H), 4.46 (s, 2H), 6.82 (s, 2H), 6.96 (dd, J=7.4, 5.0 Hz, 1H), 7.23-7.26 (m, 1H), 7.47 (td, J=6.0, 1.7 Hz, 1H), 8.32 (d br, J=5.0 Hz, 1H); LC/MS (APCI) m/z 272 (M⁺+H); HPLC tᵣ=2.56 min (98%) in 10% water-acetonitrile.

### Example 74

### 2-(1-Methyl-1H-imidazol-2-ylsulfanyl)-1-(2,4,6-trimethyl-phenyl)-ethanone

Et₃N (0.239 mL, 0.98 mmol) was added to a solution of 2-bromo-1-(2,4,6-trimethyl-phenyl)-ethanone (208 mg, 0.86 mmol) and 2-mercapto-1-methylimidazole (98 mg, 0.86 mmol) in CH₃CN (8 mL) at room temperature. The reaction was stirred overnight at room temperature then was quenched with of a saturated solution of NH₄Cl. The extraction was conducted with DCM (×2) then the organic phase was washed with brine and dried over MgSO₄. The crude residue was the purified by flash chromatography (hexane/EtOAc 0-40% gradient) to afford the expected compound (229 mg, 97%) as yellow oil. TLC single spot at R_{f} 0.23 (hexane/EtOAc 6:4); ¹H NMR (270 MHz, CDCl₃): δ2.10 (s, 6H), 2.25 (s, 3H), 4.32 (s, 2H), 6.79 (s, 2H), 6.87 (s br, 1H), 7.00 (s br, 1H); LC/MS (APCI) m/z 275 (M⁺+H); HPLC tᵣ=1.99 min (99.5%) in 10% water-acetonitrile.

### Example 75

### 2-(Pyridine-2-sulfonyl)-1-(2,4,6-trimethyl-phenyl)-ethanone

m-CPBA (125 mg, 0.56 mmol) was added neat to a solution of 1-mesityl-2-(pyridin-2-ylthio)ethanone (53 mg, 0.19 mmol) in DCM (10 mL) at room temperature and was stirred over night. The reaction was quenched by addition of a saturated solution of NaHCO₃ (10 mL), extracted with DCM, washed with brine, dried over MgSO₄ before filtration and concentration under reduced pressure. The crude mixture was purified by flash chromatography on silica gel using a gradient of 0-30% EtOAc in petroleum ether to give the expected compound (47.5 mg, 62%) as white solid. TLC single spot at R_{f} 0.31 (PE/EtOAc 7:3); Mp=[95.7-101.8° C.]; ¹H NMR (270 MHz, CDCl₃): δ 2.19 (s, 6H), 2.23 (s, 3H), 4.89 (s, 1H), 6.77 (s, 2H), 7.50-7.57 (m, 1H), 7.95 (td, J=7.7, 1.4 Hz, 1H), 8.04-8.10 (m, 1H), 8.69 (dt, J=4.7, 0.8 Hz, 1H); LC/MS (APCI) m/z 304 (M⁺+H); HPLC tᵣ=1.90 min (93%) in 10% water-acetonitrile.

### Example 76

### 1-Adamantan-1-yl-2-(1-oxy-pyridin-2-ylmethanesulfonyl)-ethanone

To a solution of 1-adamantan-1-yl-2-(pyridin-2-ylmethanesulfinyl)-ethanone (1.7 g, 5.65 mmol) in DCM (100 mL) was added mCPBA (2.44 g, purity 60-77%). The mixture was stirred at rt for 12 h, partitioned between DCM and 5% sodium carbonate solution. The organic phase was washed with brine, dried over MgSO₄ and concentrated in vacuo. Purification with flash column (methanol-DCM; gradient elution) yielded a white solid (700 mg, 35%). mp 225-227.5° C.; TLC single spot at R_{f}: 0.46 (5% CH₃OH/DCM); ¹H NMR (270 MHz, CDCl₃) δ 1.60-1.77 (6H, m, 3×CH₂), 1.82 (6H, d, J=2.7 Hz, 3×CH₂), 2.05 (3H, broad, 3×CH), 4.60 (2H, s, CH₂), 4.93 (2H, s, CH₂), 7.27-7.33 (2H, m, ArH), 7.46 (1H, dd, J=5.5, 4.4 Hz, ArH) and 8.21 (1H, dd, J=4.4, 3.0 Hz, ArH); LC/MS (ESI) m/z 350 (M⁺+H); tᵣ=1.65 min in 10% water-methanol; HRMS (FAB+) calcd. for C₁₈H₂₄NO₄S (M⁺+H) 350.1426. found 350.1414; HPLC tᵣ=1.80 min (>99%) in 10% water-acetonitrile.

### Example 77

### 1-Adamantan-1-yl-2-(4-methyl-4H-[1,2,4]triazole-3-sulfonyl)-ethanone

To a solution of 1-adamantan-1-yl-2-(4-methyl-4H-[1,2,4]triazole-3-sulfanyl)-ethanone (250 mg, 0.86 mmol) in DCM (20 mL) was added mCPBA (400 mg, purity 60-77%). The mixture was stirred at rt for 12 h, partitioned between DCM and 5% sodium carbonate solution. The organic phase was washed with brine, dried over MgSO₄ and concentrated in vacuo. Purification with flash column (EtOAc-DCM; gradient elution) yielded a white solid (85 mg, 31%). mp 149-150° C.; TLC single spot at R_{f}: 0.51 (30% EtOAc/DCM); ¹H NMR (270 MHz, CDCl₃) δ 1.60-1.80 (12H, m, 6×CH₂), 2.05 (3H, br, 3×CH), 4.02 (3H, s, CH₃), 4.71 (2H, s, CH₂) and 8.17 (1H, s, ArH); LC/MS (ESI) m/z 322 (M⁺−H); tᵣ=1.61 min in 10% water-methanol; HPLC tᵣ=1.65 min (97%) in 10% water-acetonitrile.

### Example 78

### 1-Adamantan-1-yl-2-(5-methyl-[1,3,4]thiadiazol-2-ylsulfanyl)-ethanone

To a solution of adamantan-1-yl bromomethyl ketone (643 mg, 2.5 mmol) in acetonitrile (20 mL) was added 5-methyl-1,3,4-thiadiazole-2-thiol (331 mg, 2.5 mmol), followed by triethylamine (1 mL). The mixture was stirred at ambient temperature overnight, partitioned between ethyl acetate and saturated sodium carbonate. The organic phase was washed with brine, dried over MgSO₄ and concentrated in vacuo. Purification with flash column (DCM-ethyl acetate; gradient elution) yielded the title compound as white solid (670 mg, 87%). mp 102-105° C.; TLC single spot at R_{f}: 0.37 (8% EtOAc/DCM); ¹H NMR (270 MHz, CDCl₃) δ 1.65-1.80 (6H, m, 3×CH₂), 1.90 (6H, d, J=2.8 Hz, 3×CH₂), 2.06 (3H, br, 3×CH), 2.69 (3H, s, CH₃) and 4.48 (2H, s, CH₂); LC/MS (ESI) m/z 309 (M⁺+H); tᵣ=2.58 min in 10% water-methanol; HRMS (ESI) calcd. for C₁₅H₂₀N₂OS₂Na (M⁺+Na) 331.0915. found 331.0873; HPLC tᵣ=2.67 min (>99%) in 10% water-acetonitrile.

### Example 79

### 1-Adamantan-1-yl-2-(5-methyl-[1,3,4]thiadiazole-2-sulfonyl)-ethanone

### Example 80

### 1-Adamantan-1-yl-2-(5-methyl-[1,3,4]thiadiazole-2-sulfinyl)-ethanone

To a cold (−5° C.) solution of 1-adamantan-1-yl-2-(5-methyl-[1,3,4]thiadiazol-2-ylsulfanyl)-ethanone (532 mg, 1.72 mmol) in DCM (20 mL) was added mCPBA (517 mg, purity 60-77%). The mixture was stirred at −5° C. for 1.5 h, partitioned between DCM and 5% sodium carbonate solution. The organic phase was washed with brine, dried over MgSO₄ and concentrated in vacuo. Purification with flash column (EtOAc-DCM; gradient elution) yielded Example 79 as white solid (60 mg, 10%). mp 111-113° C.; TLC single spot at R_{f}: 0.69 (12% EtOAc/DCM); ¹H NMR (270 MHz, CDCl₃) δ 1.65-1.75 (6H, m, 3×CH₂), 1.76 (6H, d, J=2.7 Hz, 3×CH₂), 2.06 (3H, br, 3×CH), 2.89 (3H, s, CH₃) and 4.75 (2H, s, CH₂); LC/MS (ESI) m/z 341 (M⁺+H); tᵣ=1.95 min in 10% water-methanol; HPLC tᵣ=2.02 min (95%) in 10% water-acetonitrile.

Example 80 was obtained as white solid (310 mg, 56%). TLC single spot at R_{f}: 0.37 (12% EtOAc/DCM); ¹H NMR (270 MHz, CDCl₃) δ 1.66-1.79 (6H, m, 3×CH₂), 1.82 (6H, d, J=2.7 Hz, 3×CH₂), 2.06 (3H, br, 3×CH), 2.85 (3H, s, CH₃) and 4.44 (2H, q, J=15 Hz, CH₂); LC/MS (ESI) m/z 325 (M⁺+H); tᵣ=2.00 min in 20% water-methanol; HPLC tᵣ=2.00 min (99%) in 10% water-acetonitrile.

### Example 81

### 1-Adamantan-1-yl-2-(pyridin-4-ylmethylsulfanyl)-ethanone

To a solution of 1-(adamantan-1-yl)-2-(acetylsulfanyl)ethan-1-one (920 mg, 3.6 mmol) in acetone (10 mL) was added 1N NaOH (4.0 mL, 4.0 mmol)). The mixture was stirred at rt under nitrogen for 1 h, added to a solution of 4-picolyl chloride (591 mg, 5.7 mmol) in CH₃CN-Et₃N (10 mL-4 mL). The mixture was stirred at rt for 24 h, partitioned between EtOAc and brine. The organic phase was washed brine, dried over MgSO₄ and concentrated in vacuo. Purification with flash column (EtOAc-DCM, gradient elution) gave product (205 mg, 19%) as a yellow oil. TLC single spot at R_{f}: 0.33 (20% EtOAc/DCM); ¹H NMR (270 MHz, CDCl₃) δ 1.62-1.75 (6H, m, 3×CH₂), 1.78 (6H, d, J=2.7 Hz, 3×CH₂), 2.01 (3H, br, 3×CH), 3.17 (2H, s, CH₂), 3.66 (2H, s, CH₂), 7.24 (2H, d, J=5.1 Hz, ArH) and 8.53 (2H, d, J=5.1 Hz, ArH); LC/MS (ESI) m/z 302 (M⁺+H), tᵣ=2.90 min in 10% water-methanol; HPLC tᵣ=3.06 min (95%) in 10% water-acetonitrile.

### Example 82

### 1-mesityl-2-(pyridin-2-ylsulfinyl)ethanone

To a solution of 1-mesityl-2-(pyridin-2-ylthio)ethanone (105 mg, 0.39 mmol) in DCM (10 mL) was added m-CPBA (95.6 mg, 0.43 mmol) at −10° C. The mixture was stirred for 10 min at −10° C. before being quenched with a saturated solution of NaHCO₃, extracted with DCM (×2), washed with water and brine, dried over MgSO₄ and concentrated in vacuum. The crude was purified by flash chromatography on silica gel using a gradient of 0-40% ethyl acetate in petroleum ether to give the expected compound (91 mg, 82%) as yellow oil. TLC single spot at R_{f} 0.19 (EtOAc/PE 4:6); ¹H NMR (CDCl₃, 270 MHz) δ2.25 (s, 9H), 4.20 (d, J=15.7 Hz, 1H), 4.52 (d, J=15.8 Hz, 1H), 6.81 (s, 2H), 7.34-7.39 (m, 1H), 7.92 (td, J=7.4, 1.6 Hz, 1H), 8.01 (d, J=8.0 Hz, 1H), 8.58 (d, J=4.7 Hz, 1H); LC/MS (APCI) m/z 288 (M⁺+H); Accurate Mass: calculated (M⁺+H) 288.1053. found 288.1052; HPLC tᵣ=1.84 min (96%) in 10% water-acetonitrile.

### Example 83

### 1-Adamantan-1-yl-2-(pyridin-4-ylmethanesulfonyl)-ethanone

### Example 84

### 1-Adamantan-1-yl-2-(pyridin-4-ylmethanesulfinyl)-ethanone

To a cold (−5° C.) solution of 1-adamantan-1-yl-2-(pyridin-4-ylmethylsulfanyl)-ethanone (120 mg, 0.4 mmol) in DCM (12 mL) was added mCPBA (120 mg, purity 60-77%). The mixture was stirred at −5° C. for 1 h, partitioned between DCM and 5% sodium carbonate solution. The organic phase was washed with brine, dried over MgSO₄ and concentrated in vacuo. Purification with flash column (CH₃OH-DCM; gradient elution) yielded Example 83 as pale grey solid (35 mg, 26%). mp 119-123° C.; TLC single spot at R_{f}: 0.67 (10% CH₃OH/DCM); ¹H NMR (270 MHz, CDCl₃) δ 1.62-1.78 (12H, m, 6×CH₂), 2.07 (3H, br, 3×CH), 3.88 (2H, s, CH₂), 4.51 (2H, s, CH₂), 7.42 (2H, dd, J=4.4, 1.3 Hz, ArH) and 8.64 (2H, dd, J=4.4, 1.3 Hz, ArH); LC/MS (ESI) m/z 334 (M⁺+H); tᵣ=1.85 min in 10% water-methanol

HRMS (ESI) calcd. for C₁₈H₂₄NO₃S (M⁺+H) 334.1477. found 334.1443; HPLC tᵣ=1.95 min (98%) in 10% water-acetonitrile.

Example 84 was obtained as a white solid (62 mg, 49%). TLC single spot at R_{f}: 0.51 (10% CH₃OH/DCM); ¹H NMR (270 MHz, CDCl₃) δ 1.62-1.76 (12H, m, 6×CH₂); 2.05 (3H, br, 3×CH), 3.57 (1H, d, J=16 Hz, CH), 3.96 (1H, d, J=16 Hz, CH), 4.00 (1H, d, J=14 Hz, CH), 4.24 (1H, d, J=13 Hz, CH), 7.24 (2H, dd, J=4.5, 1.6 Hz, ArH) and 8.63 (2H, dd, J=4.4, 1.6 Hz, ArH); LC/MS (ESI) m/z 318 (M⁺+H); tᵣ=1.80 min in 10% water-methanol; HRMS (ESI) calcd. for C₁₈H₂₄NO₂S (M⁺+H) 318.1528. found 318.1510; HPLC tᵣ=1.92 min (99%) in 10% water-acetonitrile.

### Example 85

### 2-((4-methoxypyridin-3-yl)methoxy)-1-adamantylethanone

A mixture of (4-methoxypyridin-3-yl)methanol (178 mg, 1.28 mmol), 1-adamantyl bromomethylketone (275 mg, 1.07 mmol) and potassium carbonate (888 mg, 6.43 mmol) in acetone (8 mL) was stirred at room temperature over 72 h. The mixture quenched with water, extracted with EtOAc (×2), washed with water (×2) and brine, dried over MgSO₄ and concentrated under vacuum. The crude was purified by flash chromatography on silica gel using a gradient of 0-5% methanol in DCM) to give the expected product (96.5 mg, 29%) as off-white slurry solid. TLC single spot at R_{f} 0.49 (MeOH/DCM 5:95); ¹H NMR (CDCl₃, 270 MHz) δ: 1.59-1.75 (m, 6H), 1.77 (d, J=2.8 Hz, 6H), 1.99 (s br, 3H), 3.89 (s, 3H), 4.26 (s, 2H), 4.46 (s, 2H), 6.71 (d, J=8.5 Hz, 1H), 7.63 (dd, J=8.5, 2.5 Hz, 1H), 8.06 (d, J=2.5 Hz, 1H); LC/MS (APCI) m/z 316 (M⁺+H); Accurate Mass: calculated (M⁺+H) 316.1907. found 316.1892; HPLC tᵣ=2.89 min (94%) in 10% water-acetonitrile.

### Example 86

### 2-((3-Methoxypyridin-2-yl)methoxy)-1-adamantylethanone

A mixture of (3-methoxypyridin-2-yl)methanol (128 mg, 0.92 mmol), 1-adamantyl bromomethylketone (198 mg, 0.77 mmol) and caesium carbonate (500 mg, 1.53 mmol) in acetone (10 mL) was stirred at room temperature for 15 h. The mixture was filtered, concentrated under vacuum and purified by flash chromatography (0-25%-50% Petrol ether/EtOAc) to give the expected product (161 mg, 66.2% yield) as clear yellow oil. Single TLC spot at R_{f}0.6 (Pet. ether/EtOAc 5:5); ¹H NMR (CDCl₃, 270 MHz): δ1.63-1.77 (m, 6H), 1.83 (br s, 6H), 2.03 (br s, 3H), 3.88-3.91 (m, 3H), 4.42-4.46 (m, 2H), 4.57 (d, J=3.0 Hz, 2H), 6.62 (dd, J=8.4, 2.9 Hz, 1H), 7.00-7.06 (m, 1H), 7.57-7.60 (m, 1H); LC/MS (ESI) t=2.20 min m/z 316 (M⁺+H); HPLC t=3.05 min (91.83%) in 10% water-acetonitrile.

### Example 87

### 1-(Adamantan-1-yl)-2-[(4,5-dimethyl-1,2,4-triazol-3-yl)sulfanyl]ethanone

To a solution of adamantan-1-yl bromomethyl ketone (298 mg, 1.16 mmol) in acetonitrile (8 mL) was added 4,5-dimethyl-4H-1,2,4-triazole-3-thiol (150 mg, 1.16 mmol), followed by triethylamine (1 mL). The mixture was stirred at ambient temperature overnight, partitioned between ethyl acetate and saturated sodium carbonate. The organic phase was washed with brine, dried over MgSO₄ and concentrated in vacuo to give the crude product. Purification with flash column (DCM-methanol; gradient elution) yielded the title compound as a white solid (190 mg, 54%). mp 113-114° C.; TLC single spot at R_{f}: 0.20 (8% CH₃OH/DCM); ¹H NMR (270 MHz, CDCl₃) δ 1.62-1.80 (m, 6H, 3×CH₂), 1.85 (d, J=2.8 Hz, 6H, 3×CH₂), 2.15 (broad, 3H, 3×CH), 2.39 (s, 3H, CH₃), 3.49 (s, 3H, CH₃) and 4.39 (s, 2H, CH₂); LC/MS (ESI) m/z 306 (M+H)⁺; tᵣ=1.58 min in 10% water-methanol; HRMS (ESI) calcd. for C₁₆H₂₄N₃OS (M+H)⁺ 306.1640. found 306.1627; HPLC tᵣ=1.82 min (>99%) in 10% water-acetonitrile.

### Example 88

### 1-(Adamantan-1-yl)-2-[(5-amino-1,3,4-thiadiazol-2-yl)sulfanyl]ethanone

To a solution of adamantan-1-yl bromomethyl ketone (463 mg, 1.8 mmol) in acetonitrile (20 mL) was added 5-amino-1,3,4-thiadiazole-2-thiol (266 mg, 2.0 mmol), followed by triethylamine (2 mL). The mixture was stirred at ambient temperature overnight, partitioned between DCM and brine. The organic phase was washed with brine, dried over MgSO₄ and concentrated in vacuo to give the crude product. Purification with flash column (DCM-ethyl acetate; gradient elution) yielded the title compound as a white solid (504 mg, 91%). mp 198-199° C.; TLC single spot at R_{f}: 0.29 (20% EtOAc/DCM); ¹H NMR (270 MHz, CDCl₃) δ 1.62-1.80 (m, 6H, 3×CH₂), 1.85 (d, J=2.8 Hz, 6H, 3×CH₂), 2.15 (broad, 3H, 3×CH), 2.39 (s, 3H, CH₃), 3.49 (s, 3H, CH₃) and 4.39 (s, 2H, CH₂); LC/MS (ESI) m/z 310 (M+H)⁺; tᵣ=1.83 min in 5% water-methanol; HRMS (ESI) calcd. for C₁₄H₂₀N₃OS₂ (M+H)⁺ 310.1048. found 310.1037; HPLC tᵣ=1.96 min (>99%) in 10% water-acetonitrile.

### Example 89

### 1-(Adamantan-1-yl)-2-[(5-phenyl-1H-1,2,4-triazol-3-yl)sulfanyl]ethanone

To a solution of adamantan-1-yl bromomethyl ketone (386 mg, 1.5 mmol) in acetonitrile (20 mL) 5-phenyl-1H-1,2,4-triazole-3-thiol (284 mg, 1.6 mmol) was added, followed by triethylamine (1.5 mL). The mixture was stirred at ambient temperature overnight, partitioned between DCM and brine. The organic phase was washed with brine, dried over MgSO₄ and concentrated in vacuo to give the crude product. Purification with flash column (DCM-methanol; gradient elution) yielded the title compound as a white solid (430 mg, 81%). mp 187-188° C.; TLC single spot at R_{f}: 0.52 (8% CH₃OH/DCM); ¹H NMR (270 MHz, CDCl₃) δ 1.62-1.82 (6H, m, 3×CH₂), 1.90 (6H, d, J=2.7 Hz, 3×CH₂), 2.08 (3H, br, 3×CH), 4.19 (2H, s, CH₂), 7.43 (3H, m, ArH), 8.00 (2H, m, ArH) and 11.5 (1H, br, NH); HRMS (ESI) calcd. for C₂₀H₂₄N₃OS (M+H)⁺ 354.1640. found 354.1627; LC/MS (ESI) m/z 354 (M+H)⁺; tᵣ=2.54 min in 10% water-methanol; HPLC tᵣ=2.36 min (99%) in 10% water-acetonitrile.

### Example 90

### 1-(Adamantan-1-yl)-2-{[5-(furan-2-yl)-1H-1,2,4-triazol-3-yl]sulfanyl}ethanone

To a solution of adamantan-1-yl bromomethyl ketone (386 mg, 1.5 mmol) in acetonitrile (20 mL) 5-(furan-2-yl)-1H-1,2,4-triazole-3-thiol (268 mg, 1.6 mmol) was added, followed by triethylamine (1.5 mL). The mixture was stirred at ambient temperature overnight, partitioned between DCM and brine. The organic phase was washed with brine, dried over MgSO₄ and concentrated in vacuo to give the crude product. Purification with flash column (DCM-methanol; gradient elution) yielded the title compound as a white solid (470 mg, 91%). mp 177-178° C.; TLC single spot at R_{f}: 0.49 (8% CH₃OH/DCM); ¹H NMR (270 MHz, CDCl₃) δ 1.61-1.82 (6H, m, 3×CH₂), 1.89 (6H, d, J=2.8 Hz, 3×CH₂), 2.01 (3H, br, 3×CH), 4.21 (2H, s, CH₂), 6.50 (1H, dd, J=3.6, 1.9 Hz, ArH), 6.98 (1H, d, J=3.6 Hz, ArH), and 7.49 (1H, d, J=1.9 Hz, ArH); HRMS (ESI) calcd. for C₁₈H₂₂N₃O₂S (M+H)⁺ 344.1432. found 344.1421; LC/MS (ESI) m/z 344 (M+H)⁺; tᵣ=2.15 min in 10% water-methanol; HPLC tᵣ=1.99 min (97%) in 10% water-acetonitrile.

### Example 91

### N-(5-{[2-(Adamantan-1-yl)-2-oxoethyl]sulfanyl}-1,3,4-thiadiazol-2-yl)acetamide

To a solution of 1-(adamantan-1-yl)-2-[(5-amino-1,3,4-thiadiazol-2-yl)sulfanyl]ethanone (225 mg, 0.73 mmol) in AcOH (2 mL) acetic anhydride (0.1 mL) was added. After stirring at ambient temperature for 16 h, the reaction was quenched with water and the precipitate was collected, washed with water and dried in vacuo. The title compound was obtained as a white solid (228 mg, 89%). mp 208-210° C.; TLC single spot at R_{f}: 0.66 (8% CH₃OH/DCM); ¹H NMR (300 MHz, CDCl₃) δ 1.66-1.82 (6H, m, 3×CH₂), 1.81 (6H, d, J=2.7 Hz, 3×CH₂), 2.12 (3H, br, 3×CH), 2.41 (3H, s, CH₃) and 4.38 (2H, s, CH₂); HRMS (ESI) calcd. for C₁₆H₂₂N₃O₂S₂ (M+H)⁺ 352.1153. found 352.1131; LC/MS (ESI) m/z 352 (M+H)⁺; tᵣ=1.98 min in 10% water-methanol; HPLC tᵣ=1.94 min (>99%) in 10% water-acetonitrile.

### Example 92

### 1-(Adamantan-1-yl)-2-[(1-methyl-1,2,3,4-tetrazol-5-yl)sulfanyl]ethanone

To a solution of adamantan-1-yl bromomethyl ketone (463 mg, 1.8 mmol) in acetonitrile (20 mL) 1-methyl-1H-tetrazole-5-thiol (232 mg, 2.0 mmol) was added, followed by triethylamine (1 mL). The mixture was stirred at ambient temperature overnight, partitioned between ethyl acetate and saturated sodium carbonate. The organic phase was washed with brine, dried over MgSO₄ and concentrated in vacuo to give the crude product. Purification with flash column (DCM-ethyl acetate; gradient elution) yielded the title compound as a white solid (395 mg, 75%). mp 112-113° C.; TLC single spot at R_{f}: 0.69 (10% EtOAc/DCM); ¹H NMR (270 MHz, CDCl₃) δ 1.62-1.80 (6H, m, 3×CH₂), 1.88 (6H, d, J=2.8 Hz, 3×CH₂), 2.07 (3H, broad, 3×CH), 3.97 (3H, s, CH₃) and 4.53 (2H, s, CH₂); LC/MS (ESI) m/z 293 (M+H)⁺; tᵣ=1.76 min in 10% water-methanol; HRMS (ESI) calcd. for C₁₄H₂₁N₄OS (M+H)⁺ 293.1436. found 293.1417; HPLC tᵣ=2.01 min (>99%) in 10% water-acetonitrile.

### Example 93

### N-(5-{[2-(Adamantan-1-yl)-2-oxoethane]sulfonyl}-1,3,4-thiadiazol-2-yl)acetamide

### Example 94

### N-(5-{[2-(Adamantan-1-yl)-2-oxoethane]sulfinyl}-1,3,4-thiadiazol-2-yl)acetamide

To a cold (−5° C.) solution of N-(5-{[2-(adamantan-1-yl)-2-oxoethyl]sulfanyl}-1,3,4-thiadiazol-2-yl)acetamide (170 mg, 0.484 mmol) in DCM (15 mL) mCPBA (150 mg was added, purity 60-77%). The mixture was stirred at −5° C. for 1.5 h, partitioned between DCM and 5% sodium carbonate solution. The organic phase was washed with brine, dried over MgSO₄ and concentrated in vacuo. Purification with flash column (EtOAc-DCM; gradient elution) yielded the title compound as a white solid (20 mg, 11%). mp 216-218° C.; TLC single spot at R_{f}: 0.52 (70% EtOAc/DCM); ¹H NMR (270 MHz, CDCl₃) δ 1.62-1.80 (m, 12H, 6×CH₂), 2.07 (br, 3H, 3×CH), 2.47 (s, 3H, CH₃) and 4.67 (s, 2H, CH₂); LC/MS (ESI) m/z 384 (M+H)⁺; tᵣ=1.51 min in 10% water-methanol; HRMS (ESI) calcd. for C₁₆H₂₂N₃O₄S₂ (M+H)⁺ 384.1051. found 384.1087; HPLC tᵣ=1.68 min (>99%) in 10% water-acetonitrile. Ex. 94 was obtained as white solid (89 mg, 50%). TLC single spot at R_{f}: 0.43 (70% EtOAc/DCM); ¹H NMR (270 MHz, CDCl₃) δ 1.65-1.85 (m, 12H, 6×CH₂), 2.06 (br, 3H, 3×CH), 2.45 (s, 3H, CH₃), 4.36 (d, J=16 Hz, 1H, CH) and 4.48 (d, J=16 Hz, 1H, CH); LC/MS (ESI) m/z 368 (M+H)⁺; tᵣ=1.65 min in 10% water-methanol; HRMS (ESI) calcd. for C₁₆H₂₂N₃O₃S₂ (M+H)⁺ 368.1102. found 368.1087; HPLC tᵣ=1.63 min (98%) in 10% water-acetonitrile.

### Example 95

### N-(5-{[2-(Adamantan-1-yl)-2-oxoethyl]sulfanyl}-1,3,4-thiadiazol-2-yl)cyclopropanecarboxamide

To a solution of 1-(adamantan-1-yl)-2-[(5-amino-1,3,4-thiadiazol-2-yl)sulfanyl]ethanone (440 mg, 1.43 mmol) in DCM (20 mL) pyridine (0.4 mL) was added, followed by cyclopropanecarbonyl chloride (0.14 mL, 1.5 mmol). After stirred at ambient temperature for 24 h, the reaction was quenched with water and extracted with DCM. The organic phase was washed with brine, dried over MgSO₄ and concentrated in vacuo. Purification with flash column (EtOAc-DCM; gradient elution) yielded the title compound as a white solid (430 mg, 79%). mp 218-219° C.; TLC single spot at R_{f}: 0.38 (10% EtOAc/DCM); ¹H NMR (300 MHz, CDCl₃) δ 1.00 (m, 2H, CH₂), 1.22 (m, 2H, CH₂), 1.65-1.80 (m, 6H, 3×CH₂), 1.85 (d, J=2.7 Hz, 6H, 3×CH₂), 2.39 (m, 4H, 4×CH) and 4.35 (s, 2H, CH₂); LC/MS (ESI) m/z 378 (M+H)⁺; tᵣ=2.51 min in 10% water-methanol; HRMS (ESI) calcd. for C₁₈H₂₃N₃O₂S₂ (M+H)⁺ 378.1310. found 378.1290; HPLC tᵣ=2.22 min (98%) in 10% water-acetonitrile.

### Example 96

### N-(5-{[2-(Adamantan-1-yl)-2-oxoethane]sulfinyl}-1,3,4-thiadiazol-2-yl)cyclopropanecarboxamide

To a cold (−5° C.) solution of N-(5-{[2-(adamantan-1-yl)-2-oxoethyl]sulfanyl}-1,3,4-thiadiazol-2-yl)cyclopropanecarboxamide (330 mg, 0.88 mmol) in DCM (30 mL) mCPBA (273 mg, purity 60-77%) was added. The mixture was stirred at −5° C. for 45 min, partitioned between DCM and 5% sodium carbonate solution. The organic phase was washed with brine, dried over MgSO₄ and concentrated in vacuo. Purification with flash column (EtOAc-DCM; gradient elution) yielded the title compound as a white solid (190 mg, 55%). mp 172.5-174° C.; TLC single spot at R_{f}: 0.33 (15% EtOAc/DCM); ¹H NMR (270 MHz, CDCl₃) δ 1.12 (m, 2H, CH₂), 1.25 (m, 2H, CH₂), 1.62-1.82 (m, 12H, 6×CH₂), 2.05 (br, 4H, 4×CH), 4.36 (d, J=16 Hz, 1H, CH) and 4.45 (d, J=16 Hz, 1H, CH); LC/MS (ESI) m/z 394 (M+H)⁺; tᵣ=1.85 min in 10% water-methanol; HRMS (ESI) calcd. for C₁₈H₂₄N₃O₃S₂(M+H)⁺ 394.1259. found 394.1247; HPLC tᵣ=1.74 min (>99%) in 10% water-acetonitrile.

### Example 97

### N-(5-{[2-(Adamantan-1-yl)-2-oxoethane]sulfonyl}-1,3,4-thiadiazol-2-yl)cyclopropanecarboxamide

To a solution of N-(5-{[2-(adamantan-1-yl)-2-oxoethyl]sulfanyl}-1,3,4-thiadiazol-2-yl)cyclopropanecarboxamide (110 mg, 0.29 mmol) in DCM (12 mL) mCPBA (89 mg, purity 60-77%) was added. The mixture was stirred at rt for 10 h, partitioned between DCM and 5% sodium carbonate solution. The organic phase was washed with brine, dried over MgSO₄ and concentrated in vacuo. Purification with flash column (EtOAc-DCM; gradient elution) yielded the title compound as a white solid (53 mg, 45%). mp 209-211° C.; TLC single spot at R_{f}: 0.58 (25% EtOAc/DCM);

¹H NMR (270 MHz, CDCl₃) δ 1.12 (m, 2H, CH₂), 1.25 (m, 2H, CH₂), 1.60-1.80 (m, 12H, 6×CH₂), 2.08 (br, 4H, 4×CH) and 4.64 (s, 2H, CH₂); LC/MS (ESI) m/z 410 (M+H)⁺; tᵣ=1.83 min in 10% water-methanol; HRMS (ESI) calcd. for C₁₈H₂₄N₃O₄S₂ (M+H)⁺410.1208. found 410.1202; HPLC tᵣ=1.82 min (>99%) in 10% water-acetonitrile.

### Example 98

### 1-Adamantan-1-yl-2-(5-methoxy-pyridin-3-ylmethoxy)-ethanone

(5-Methoxypyridin-3-yl)methanol (69 mg, 0.49 mmol) in THF (2 mL) was added via cannula to a suspension of NaH (18 mg, 60% in mineral oil, 0.74 mmol) in THF (1.5 mL) at 0° C. The mixture was stirred at 0° C. for 30 min then 1-adamantyl bromomethylketone (126 mg, 0.49 mmol) was added via cannula in THF (2 mL). The reaction was allowed to warm up slowly to room temperature then was quenched by addition of water, extracted with EtOAc, washed with brine, dried over MgSO₄, concentrated under vacuum and purified by flash chromatography (DCM/MeOH 0-5%) to give the expected compound (43 mg, 28%) as a light yellow oil. Single TLC spot at R_{f}0.54 (DCM/MeOH 9:1); ¹H NMR (CDCl₃, 270 MHz): δ1.56-1.81 (m, 6H), 1.81 (d, J=2.7 Hz, 6H), 2.02 (br s, 3H), 3.85 (s, 3H), 4.33 (s, 2H), 4.56 (s, 2H), 7.29 (br s, 1H), 8.14 (br s, 1H), 8.24 (d, J=3.0 Hz, 1H); LC/MS (ESI) t=2.05 min, m/z 316 (M⁺+H); HPLC t=2.18 min (98.78%) in 10% water-acetonitrile.

### Example 99

### 3-(4-Chlorophenyl)-3-methyl-1-(4-methyl-4H-1,2,4-triazol-3-ylthio)butan-2-one

Triethylamine (0.739 mL, 5.30 mmol) was added to a solution of 1-bromo-3-(4-chlorophenyl)-3-methylbutan-2-one (733 mg, 2.65 mmol) and 4-methyl-4H-1,2,4-triazol-3-thiol (306 mg, 2.65 mmol) in CH₃CN (15 mL) at room temperature and the reaction was stirred for 24 h. The mixture was diluted with DCM (25 mL), washed with water, NaHCO₃ and brine then the organic phase was dried over MgSO₄, filtered and concentrated under vacuum. The crude was purified by flash chromatography (DCM/EtOAc 0-70%) to give the title compound (323 mg, 39% yield) as a yellow oil. Single TLC spot at R_{f}0.15 (DCM/EtOAC 5:5); ¹H NMR (CDCl₃, 270 MHz): δ1.53 (s, 6H), 3.59 (s, 3H), 4.13 (s, 2H), 7.13-7.22 (m, 2H), 7.27-7.35 (m, 2H), 8.06 (s, 1H); LC/MS (ESI) t=1.59 min m/z 310 (M+H)⁺; HPLC t=1.59 min (100%) in 10% water-acetonitrile.

### Example 100

### 1-(1-(4-Chlorophenyl)cyclopropyl)-2-(4-methyl-4H-1,2,4-triazol-3-ylthio)ethanone

Triethylamine (0.203 mL, 1.46 mmol) was added to a solution of 2-bromo-1-(1-(4-chlorophenyl)cyclopropyl)ethanone (200 mg, 0.73 mmol) and 4-methyl-4H-1,2,4-triazol-3-thiol (84 mg, 0.73 mmol) in CH₃CN (5 mL) at room temperature and the reaction was stirred for 15 min. The mixture was diluted with DCM (25 mL), washed with water, NaHCO₃ and brine then the organic phase was dried over MgSO₄, filtered and concentrated under vacuum. The crude was purified by flash chromatography (DCM/EtOAc 0-70%) to give the title compound (64 mg, 28% yield) as a clear oil. Single TLC spot at R_{f}0.10 (DCM/EtOAC 7:3); ¹H NMR (CDCl₃, 270 MHz): δ1.21 (q, J=3.3 Hz, 2H), 1.64 (q, J=3.6 Hz, 2H), 3.56 (s, 3H), 4.08 (s, 2H), 7.26-7.38 (m, 4H), 8.04 (s, 1H); LC/MS (ESI) t=1.54 min m/z 308 (M+H)⁺; HPLC t=1.54 min (98.92%) in 10% water-acetonitrile.

### Example 101

### 1-(Adamantan-1-yl)-2-[(5-cyclopropyl-4-methyl-4H-1,2,4-triazol-3-yl)sulfanyl]ethan-1-one

2-(Cyclopropanecarbonyl)-N-methylhydrazinecarbothioamide (700 mg, 4.05 mmol) was added to NaOH solution (2N, 5 mL). The mixture was refluxed under nitrogen for 5 h, cooled to room temperature and concentrated in vacuo. The residue was dissolved in acetonitrile (5 mL), adamantan-1-yl bromomethyl ketone (771 mg, 3.0 mmol) and then was added. The mixture was stirred at ambient temperature overnight, partitioned between DCM and water. The organic phase was washed with brine, dried over MgSO₄ and concentrated in vacuo to give the crude product. Purification with flash column (DCM-ethyl acetate; gradient elution) yielded the title compound as a white solid (390 mg, 39%). mp 96-97.5° C.; TLC single spot at R_{f}: 0.32 (30% EtOAc/DCM); ¹H NMR (270 MHz, CDCl₃) δ 1.00 (m, 4H, 2×CH₂), 1.60-1.80 (m, 7H, 3×CH₂ and CH), 1.83 (d, J=2.8 Hz, 6H, 3×CH₂), 2.01 (broad, 3H, 3×CH), 3.57 (s, 3H, CH₃) and 4.35 (s, 2H, CH₂); LC/MS (ESI) m/z 232 (M+H)⁺; tᵣ=1.98 min in 10% water-methanol; HRMS (ESI) calcd. for C₁₈H₂₆N₃O_{S} (M+H)⁺ 332.1796. found 332.1796; HPLC tᵣ=1.89 min (>99%) in 10% water-acetonitrile.

### Example 102

### 1-(Adamantan-1-yl)-2-{[4-methyl-5-(thiophen-2-yl)-4H-1,2,4-triazol-3-yl]sulfanyl}ethan-1-one

To a solution of adamantan-1-yl bromomethyl ketone (514 mg, 2.0 mmol) in acetonitrile (20 mL) 4-methyl-5-(thiophen-2-yl)-4H-1,2,4-triazole-3-thiol (395 mg, 2.0 mmol) was added, followed by triethylamine (1 mL). The mixture was stirred at ambient temperature overnight, partitioned between DCM and brine. The organic phase was washed with brine, dried over MgSO₄ and concentrated in vacuo to give the crude product. Purification with flash column (DCM-ethyl acetate; gradient elution) yielded the title compound as a white solid (395 mg, 53%). mp 156-157° C.; TLC single spot at R_{f}: 0.75 (18% EtOAc/DCM); ¹H NMR (270 MHz, CDCl₃) δ 1.66-1.82 (m, 6H, 3×CH₂), 1.88 (d, J=2.8 Hz, 6H, 3×CH₂), 2.05 (broad, 3H, 3×CH), 3.73 (s, 3H, CH₃), 4.46 (s, 2H, CH₂), 7.14 (t, J=4.9 Hz, ArH), 7.42 (d, J=4.8 Hz, ArH) and 7.47 (d, J=4.9 Hz, ArH); LC/MS (ESI) m/z 374 (M+H)⁺; tᵣ=2.13 min in 10% water-methanol; HRMS (ESI) calcd. for C₁₉H₂₄N₃OS₂ (M+H)⁺ 374.1361. found 374.1362; HPLC tᵣ=2.13 min (>99%) in 10% water-acetonitrile.

### Example 103

### 1-(Adamantan-1-yl)-2-{[5-methyl-4-(propan-2-yl)-4H-1,2,4-triazol-3-yl]sulfanyl}ethan-1-one

2-Acetyl-N-isopropylhydrazinecarbothioamide (780 mg, 4.46 mmol) was added to a NaOH solution (2N, 5 mL). The mixture was refluxed under nitrogen for 6 h, cooled to room temperature and concentrated in vacuo. The residue was dissolved in acetonitrile (15 mL), adamantan-1-yl bromomethyl ketone (900 mg, 3.5 mmol) was then added. The mixture was stirred at ambient temperature overnight, partitioned between DCM and water. The organic phase was washed with brine, dried over MgSO₄ and concentrated in vacuo to give the crude product. Purification with flash column (DCM-ethyl acetate; gradient elution) yielded the title compound as a white solid (510 mg, 44%). mp 119-120.5° C.; TLC single spot at R_{f}: 0.33 (30% EtOAc/DCM); ¹H NMR (270 MHz, CDCl₃) δ 1.49 (d, J=6.8 Hz, 6H, 2×CH₃), 1.65-1.85 (m, 6H, 3×CH₂), 1.87 (d, J=2.5 Hz, 6H, 3×CH₂), 2.03 (br s, 3H, 3×CH), 2.45 (s, 3H, CH₃) and 4.45 (m, 3H, CH and CH₂); LC/MS (ESI) m/z 334 (M+H)⁺; tᵣ=1.91 min in 10% water-methanol; HRMS (ESI) calcd. for C₁₈H₂₈N₃OS (M+H)⁺ 334.1953. found 334.1953; HPLC tᵣ=2.05 min (>99%) in 10% water-acetonitrile.

### Example 104

### 1-(Adamantan-1-yl)-2-{[5-(dimethylamino)-1,3,4-thiadiazol-2-yl]sulfanyl}ethan-1-one

To a solution of N,N-dimethylhydrazinecarbothioamide (477 mg, 4 mmol) in DMF (6 mL) triethylamine (1 mL) was added, followed by the dropwise addition of CS₂ (0.4 mL). The mixture was stirred at rt overnight and then at 60° C. for 5 h, cooled to room temperature. Adamantan-1-yl bromomethyl ketone (514 mg, 2.0 mmol) was added. The mixture was stirred at ambient temperature overnight, partitioned between ethyl acetate and brine. The organic phase was washed with brine, dried over MgSO₄ and concentrated in vacuo to give the crude product. Purification with flash column (DCM-ethyl acetate; gradient elution) yielded the title compound as an off-white solid (365 mg, 54%). mp 150-151° C.; TLC single spot at R_{f}: 0.31 (25% EtOAc/DCM);

¹H NMR (270 MHz, CDCl₃) δ 1.65-1.82 (m, 6H, 3×CH₂), 1.85 (d, J=2.8 Hz, 6H, 3×CH₂), 2.03 (br, 3H, 3×CH), 3.11 (s, 6H, 2×CH₃) and 4.40 (s, 2H, CH₂); LC/MS (ESI) m/z 338 (M+H)⁺; tᵣ=2.23 min in 10% water-methanol; HRMS (ESI) calcd. for C₁₆H₂₄N₃OS₂ (M+H)⁺ 338.1361. found 338.1353; HPLC tᵣ=2.43 min (99%) in 10% water-acetonitrile.

### Example 105

### 1-(Adamantan-1-yl)-2-({4-[(4-chlorophenyl)methyl]-5-methyl-4H-1,2,4-triazol-3-yl}sulfanyl)ethan-1-one

To a solution of adamantan-1-yl bromomethyl ketone (161 mg, 0.63 mmol) in acetonitrile (5 mL) 4-(4-chlorobenzyl)-5-methyl-4H-1,2,4-triazole-3-thiol (150 mg, 0.63 mmol) was added, followed by triethylamine (0.5 mL). The mixture was stirred at ambient temperature overnight, partitioned between DCM and brine. The organic phase was washed with brine, dried over MgSO₄ and concentrated in vacuo to give the crude product. Purification with flash column (DCM-ethyl acetate; gradient elution) yielded the title compound as a white solid (195 mg, 74%). mp 158-159° C.; TLC single spot at R_{f}: 0.32 (8% CH₃OH/DCM); ¹H NMR (270 MHz, CDCl₃) δ 1.65-1.82 (m, 6H, 3×CH₂), 1.83 (d, J=2.6 Hz, 6H, 3×CH₂), 2.03 (broad, 3H, 3×CH), 2.33 (s, 3H, CH₃), 4.39 (s, 2H, CH₂), 5.06 (s, 2H, CH₂), 7.02 (d, J=8.4 Hz, ArH) and 7.31 (d, J=8.4 Hz, ArH); LC/MS (ESI) m/z 416 (M+H)⁺; tᵣ=2.32 min in 10% water-methanol; HRMS (ESI) calcd. for C₂₂H₂₇ClN₃OS (M+H)⁺ 416.1563. found 416.1547; HPLC tᵣ=2.44 min (>99%) in 10% water-acetonitrile.

### Example 106

### 1-(Adamantan-1-yl)-2-{[5-(methylsulfanyl)-1,3,4-thiadiazol-2-yl]sulfanyl}ethan-1-one

To a solution of adamantan-1-yl bromomethyl ketone (257 mg, 1.0 mmol) in acetonitrile (10 mL) 5-(methylthio)-1,3,4-thiadiazole-2-thiol (164 mg, 1.0 mmol) was added, followed by triethylamine (0.5 mL). The mixture was stirred at ambient temperature overnight, partitioned between DCM and brine. The organic phase was washed with brine, dried over MgSO₄ and concentrated in vacuo to give the crude product. Purification with flash column (DCM-ethyl acetate; gradient elution) yielded the title compound as a white solid (310 mg, 91%). mp 133.5-135° C.; TLC single spot at R_{f}: 0.87 (40% EtOAc/DCM); ¹H NMR (270 MHz, CDCl₃) δ 1.65-1.83 (m, 6H, 3×CH₂), 1.89 (d, J=2.7 Hz, 6H, 3×CH₂), 2.06 (broad, 3H, 3×CH), 2.72 (s, 3H, CH₃) and 4.47 (s, 2H, CH₂); LC/MS (ESI) m/z 341 (M+H)⁺; tᵣ=2.67 min in 10% water-methanol; HRMS (ESI) calcd. for C₁₅H₂₁N₂OS₃ (M+H)⁺ 341.0816. found 341.0807; HPLC tᵣ=3.05 min (98%) in 10% water-acetonitrile.

### Example 107

### 1-(Adamantan-1-yl)-2-{[5-(methoxymethyl)-4-methyl-4H-1,2,4-triazol-3-yl]sulfanyl}ethan-1-one

To a solution of 4-methyl-3-thiosemicarbazide (526 mg, 5.0 mmol) in DCM (10 mL) pyridine (1.2 mL) was added, followed by metholoxyacetyl chloride (0.46 mL, 5 mmol) added dropwise at 0° C. The mixture was stirred at rt overnight, concentrated in vacuo; a 2N NaOH solution (6 mL) was added. The mixture was refluxed under nitrogen for 6 h, cooled to room temperature; adamantan-1-yl bromomethyl ketone (643 mg, 2.5 mmol) was added. The mixture was stirred at ambient temperature overnight, partitioned between DCM and brine. The organic phase was washed with brine, dried over MgSO₄ and concentrated in vacuo to give the crude product. Purification with flash column (DCM-CH₃OH; gradient elution) yielded the title compound as a white solid (550 mg, 66%). mp 109-111° C.; TLC single spot at R_{f}: 0.70 (10% CH₃OH/DCM); ¹H NMR (270 MHz, CDCl₃) δ 1.60-1.82 (m, 6H, 3×CH₂), 1.86 (d, J=2.8 Hz, 6H, 3×CH₂), 2.04 (br, 4H, 4×CH), 3.34 (s, 3H, CH₃), 3.59 (s, 3H, CH₃), 4.44 (s, 2H, CH₂) and 4.58 (s, 2H, CH₂); LC/MS (ESI) m/z 336 (M+H)⁺; tᵣ=1.91 min in 10% water-methanol; HRMS (ESI) calcd. for C₁₇H₂₆N₃O₂S (M+H)⁺ 336.1745. found 336.1730; HPLC tᵣ=1.82 min (99%) in 10% water-acetonitrile.

### Example 108

### 1-(1-(4-Chlorophenyl)cyclobutyl)-2-(4-methyl-4H-1,2,4-triazol-3-ylthio)ethanone

FPC03002, STX3555, BN 115283-OO/1

C₁₅H₁₆ClN₃OS, MW 321.83

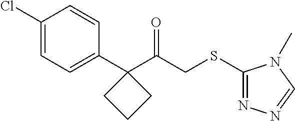

4-Methyl-4H-1,2,4-triazole-3-thiol (171 mg, 1.49 mmol) was added neat to a solution of 2-bromo-1-(1-(4-chlorophenyl)cyclobutyl)ethanone (428 mg, 1.49 mmol) in CH₃CN (10 mL) at room temperature, then Et₃N (0.42 mL, 2.98 mmol) was added neat to the mixture and was stirred 24 h. The reaction was quenched by addition of a saturated solution of NaHCO₃ then was extracted with DCM, washed with brine and dried over MgSO₄, filtered and concentrated under vacuum. The crude mixture was purified by flash chromatography (silica, DCM/EtOAc gradient 0-80%). The major product was isolated (378 mg, 79%) as a light yellow solid. R_{f} 0.13 (DCM/EtOAc 7:3); Mp=[97-101° C.]; ¹H NMR (270 MHz, CDCl₃): δ1.78-1.98 (m, 2H), 2.34-2.49 (m, 2H), 2.77-2.90 (m, 2H), 3.58 (s, 3H), 4.06 (s, 2H), 7.13-7.21 (m, 2H), 7.27-7.36 (m, 2H), 8.06 (s, 1H); LC/MS (ESI) m/z 322 (M⁺); HPLC tᵣ=1.67 min (100%) in 10% water-acetonitrile.

### Example 109

### 1-(1-(4-Chlorophenyl)cyclobutyl)-2-(4-methyl-4H-1,2,4-triazol-3-ylsulfinyl)ethanone

FPC03007B, STX3556, BN115294-OO/1

C₁₅H₁₆ClN₃O₂S, MW 337.82

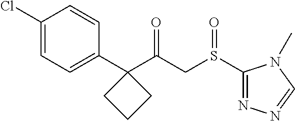

m-CPBA (96 mg, 0.43 mmol) was added neat to a solution of 1-(1-(4-chlorophenyl)cyclobutyl)-2-(4-methyl-4H-1,2,4-triazol-3-ylthio)ethanone (125 mg, 0.39 mmol) in dry DCM (5 mL) at −10° C. overnight (temperature stayed between −15 and +10° C.). The reaction was quenched by the addition of a saturated solution of NaHCO₃, extracted with DCM, washed with brine and dried over MgSO₄ filtered and reduced in vacuo. The crude mixture was purified by flash chromatography (silica, DCM/MeOH 0-10%) to give the titled sulfoxide (114 mg, 87%) as yellow oil.

R_{f}0.54 (DCM/MeOH 9:1); ¹H NMR (270 MHz, CDCl₃): δ1.80-1.97 (m, 2H), 2.30-2.50 (m, 2H), 2.73-2.94 (m, 2H), 3.91 (s, 3H), 4.49 (dd, J=117, 16 Hz, 2H), 7.13-7.19 (m, 2H), 7.31-7.38 (m, 2H), 8.18 (s, 1H); LC/MS (ESI) m/z 338 (M⁺+H); HPLC tᵣ=3.26 min (99.30%) in 10% water-acetonitrile.

### Biological Assay

### 11β-HSD1 HEK Assay Protocol (Cell Based Assay)

### Introduction

11β-HSD1 activity is measured in whole HEK 293 cells stably transfected with the HSD11B1 gene. Cells are incubated in 96-well microplates in the presence of tritiated substrate. Enzyme activity is determined by measuring the amount of tritated product by scintillation proximity assay (SPA). Assay plates contain internal high and low controls to allow calculation of percentage inhibition.

### Method

1. Each well of a 96-well culture plate is seeded with HEK 293/HSD11B1 cells in 100 μl medium.
2. When the cells are 80% confluent the medium is removed from each well. 100 μl fresh, serum-free, medium containing ³H-cortisone and test compound in 1% DMSO is added to each well*. Control wells are also dispensed. High control wells do not contain compound, while low controls do not contain cells.
3. The plate is incubated at 37° C. for the required time period.
4. 50 μl of media is removed from each well and transferred to a microplate containing 100 μl of a pre-incubated mixture of anti-cortisol antibody and SPA bead. The mixture is incubated with gentle shaking until equilibrium is reached, before transferring to a scintillation counter to establish the enzyme activity in each sample.

### Preparation of Samples

10 μl of compound is dispensed into each well of a 96-well microplate in 10% DMSO at 100 μM concentration. 90 μl media containing ³H-cortisone is added to each well. The compound/media/substrate mixture is then transferred to the assay plate containing the cells. The final concentration of compound and DMSO is 10 μM and 1% respectively.

### Inhibition Data

The structures of the above synthesised compounds and the data obtained are given in the table below

Compounds showing >60% inhibition of 11β-HSD1 at 1 μM have been designated (a).

Compounds showing from 20 to 60% inhibition of 11β-HSD1 at 1 μM have been designated (b).

Compounds showing <20% inhibition of 11β-HSD1 at 1 μM have been designated (c).

| Example | | |
|---|---|---|
| No | Activity | STRUCTURE |
| 1 | a | 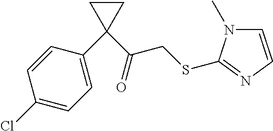 |
| 2 | c | 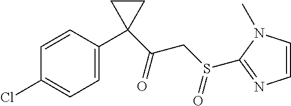 |
| 3 | c | 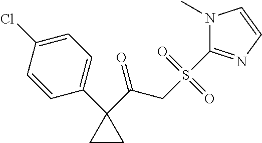 |
| 4 | c | 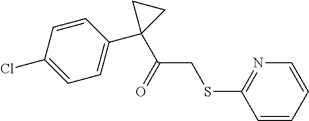 |
| 5 | b | 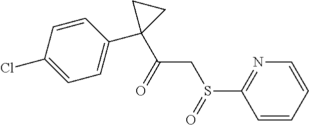 |
| 6 | c | 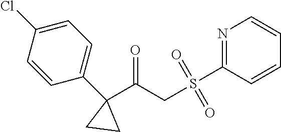 |
| 7 | a | 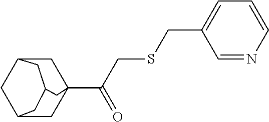 |
| 8 | a | 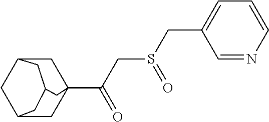 |
| 9 | a | 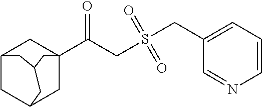 |
| 10 | a | 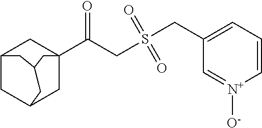 |
| 11 | a | 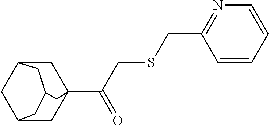 |
| 12 | a | 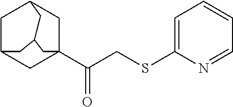 |
| 13 | b | 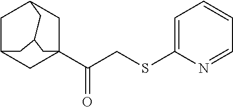 |
| 14 | c | 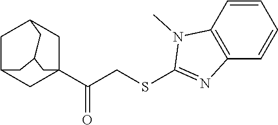 |
| 15 | c | 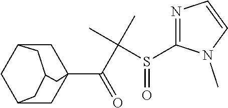 |
| 16 | a | 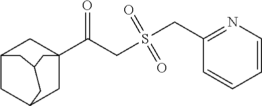 |
| 17 | a | 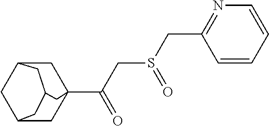 |
| 18 | a | 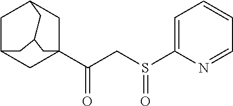 |
| 19 | a | 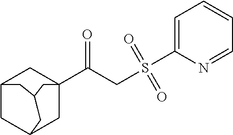 |
| 20 | b | 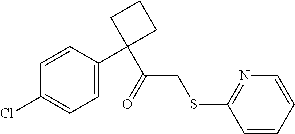 |
| 21 | b | 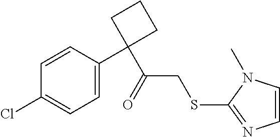 |
| 22 | c | 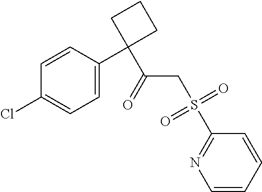 |
| 23 | c | 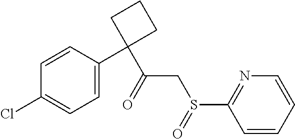 |
| 24 | c | 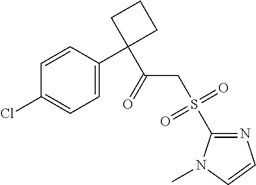 |
| 25 | c | 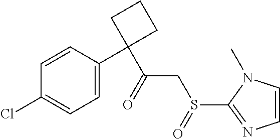 |
| 26 | a | 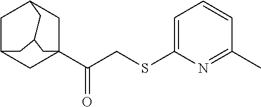 |
| 27 | a | 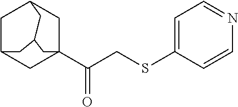 |
| 28 | a | 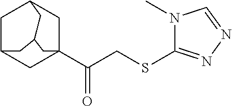 |
| 29 | a | 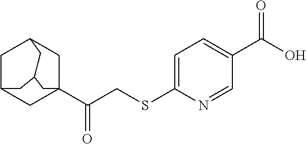 |
| 30 | a | 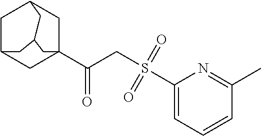 |
| 31 | a | 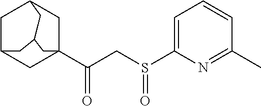 |
| 32 | a | 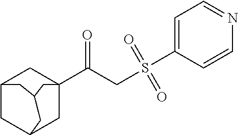 |
| 33 | a | 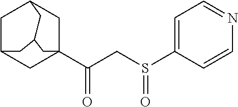 |
| 34 | a | 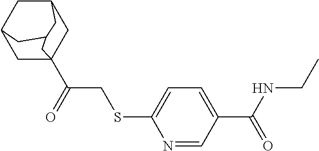 |
| 35 | a | 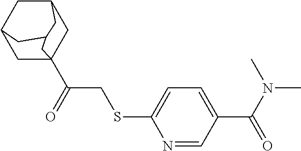 |
| 36 | a | 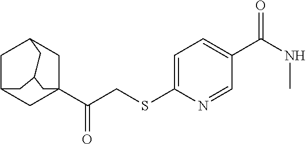 |
| 37 | a | 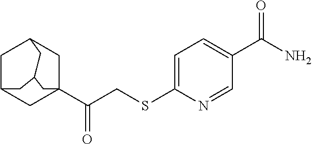 |
| 38 | a | 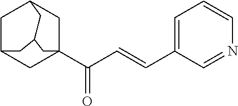 |
| 39 | a | 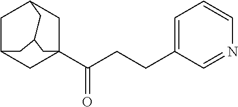 |
| 40 | b | 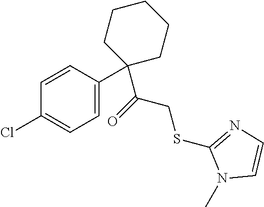 |
| 41 | b | 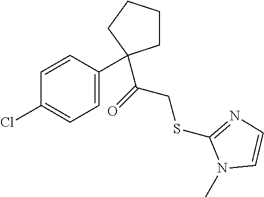 |
| 42 | c | 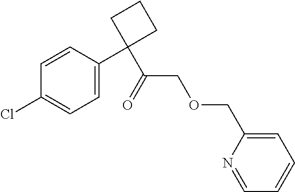 |
| 43 | b | 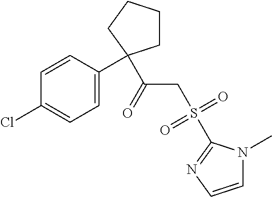 |
| 44 | b | 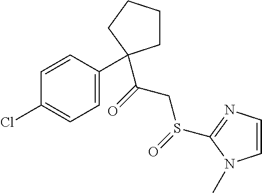 |
| 45 | b | 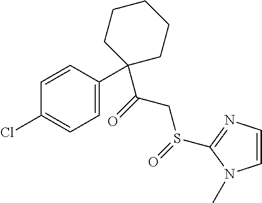 |
| 46 | b | 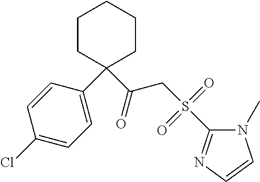 |
| 47 | b | 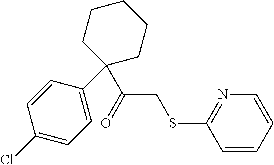 |
| 48 | b | 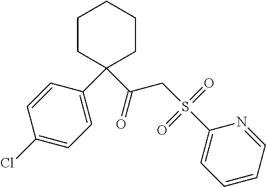 |
| 49 | c | 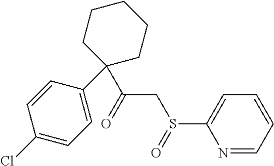 |
| 50 | a | 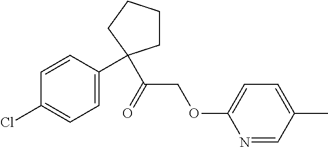 |
| 51 | b | 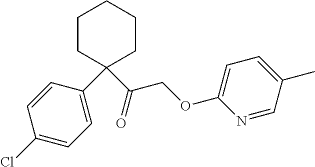 |
| 52 | a | 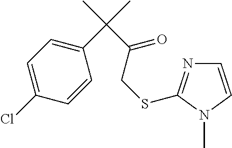 |
| 53 | b | 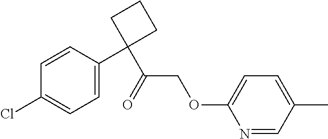 |
| 54 | a | 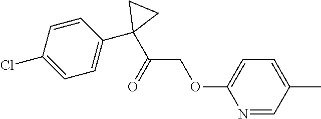 |
| 55 | b | 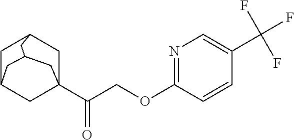 |
| 56 | b | 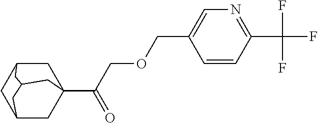 |
| 57 | b | 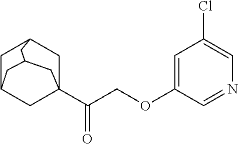 |
| 58 | b | 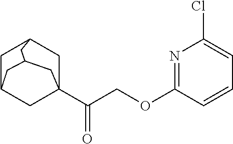 |
| 59 | b | 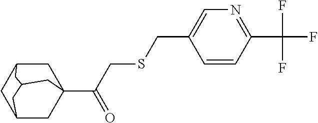 |
| 60 | a | 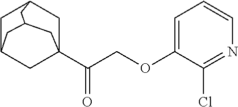 |
| 61 | a | 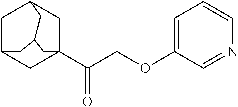 |
| 62 | b | 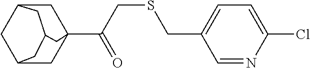 |
| 63 | a | 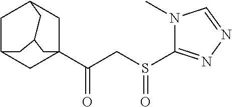 |
| 64 | a | 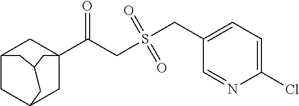 |
| 65 | a | 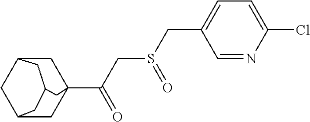 |
| 66 | a | 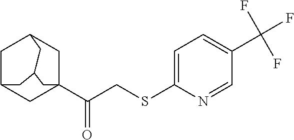 |
| 67 | b | 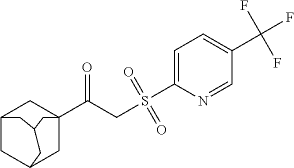 |
| 68 | b | 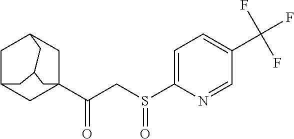 |
| 69 | b | 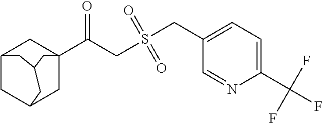 |
| 70 | c | 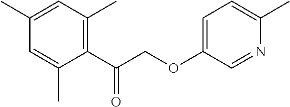 |
| 71 | a | 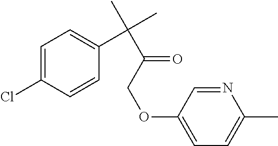 |
| 72 | c | 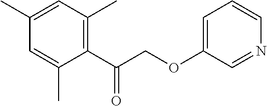 |
| 73 | c | 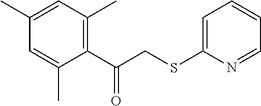 |
| 74 | c | 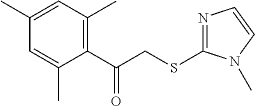 |
| 75 | c | 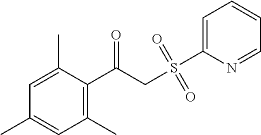 |
| 76 | a | 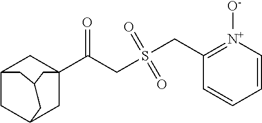 |
| 77 | a | 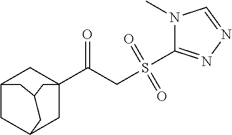 |
| 78 | a | 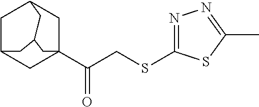 |
| 79 | a | 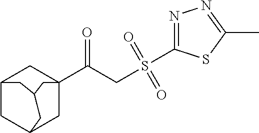 |
| 80 | a | 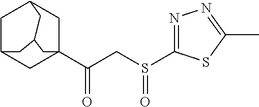 |
| 81 | a | 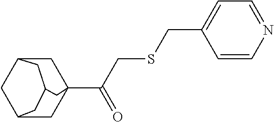 |
| 82 | c | 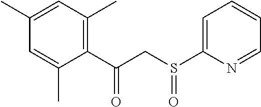 |
| 83 | a | 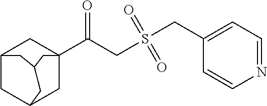 |
| 84 | a | 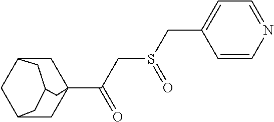 |
| 85 | a | 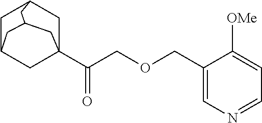 |
| 86 | a | 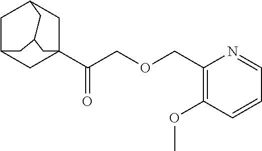 |
| 87 | a | 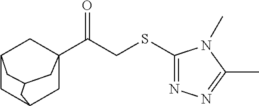 |
| 88 | a | 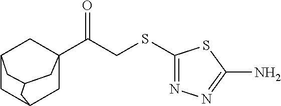 |
| 89 | c | 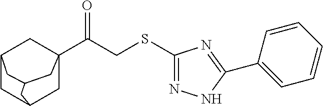 |
| 90 | c | 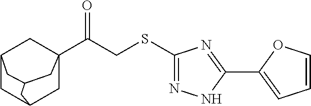 |
| 91 | a | 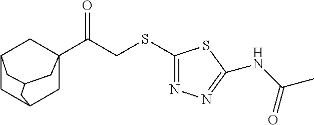 |
| 92 | a | 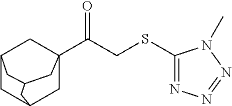 |
| 93 | a | 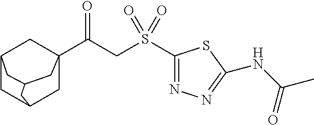 |
| 94 | a | 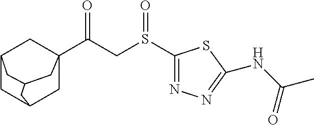 |
| 95 | b | 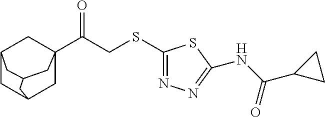 |
| 96 | a | 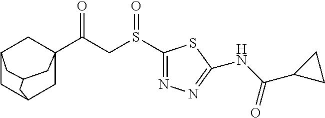 |
| 97 | a | 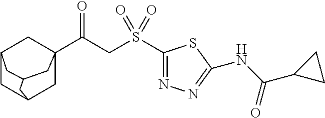 |
| 98 | a | 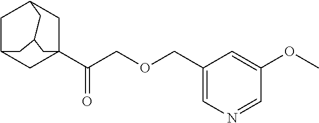 |
| 99 | b | 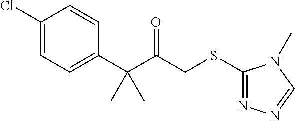 |
| 100 | b | 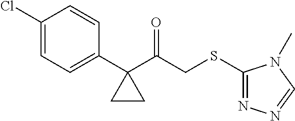 |
| 101 | a | 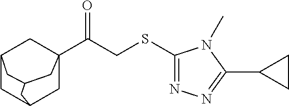 |
| 102 | a | 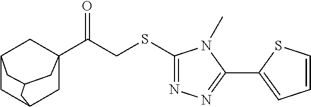 |
| 103 | a | 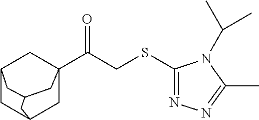 |
| 104 | a | 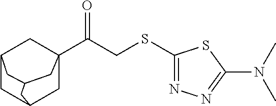 |
| 105 | b | 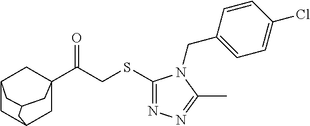 |
| 106 | a | 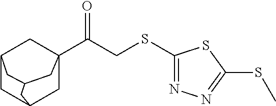 |
| 107 | a | 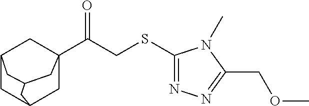 |
| 108 | b | 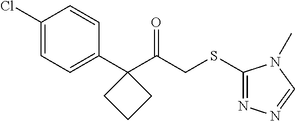 |
| 109 | c | 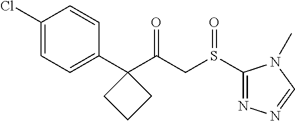 |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

### REFERENCES

1. Hammond, G H (1990): Molecular properties of corticosteroid binding globulin and sex-steroid binding proteins. Endocr. Rev. 11, 65-79.
2. Gomez-Sanchez E P, Gomex-Sanchez C E (1997): First there was one, then two . . . why not more 11 β-Hydroxysteroid Dehydrogenases? Endocrinology vol. 138, 12.
3. Krozowski Z S, Funder J W (1983): Renal mineralocorticosterone receptors and hippocampal corticosterone binding species have identical intrinsic steroid specificity. Proc. Natl. Sci. USA 80: 6056-60
4. Ulick S, Levine L S, Gunczler P, Zanconato G, Rarnirez L C, Rauh W, Rosier A, Bradlow H L, Mew M I (1979): A syndrome of apparent mineralocorticoid excess associated with defects in the peripheral metabolism of cortisol. J. Clin. Endo. And Metab. 49: 757-64.
5. Edwards C R W, Stewart P M, Burt D, Brett L, McIntyre M A, Sutanto W S, Kloet E R, Monder C (1998): Localisation of 11 (3-HSD-tissue specific protector of the mineralocorticoid receptor. Lancet 2: 986-989.
6. Moore CCD, Melloh S H, Murai I, Siiteri P K, Miller W L (1993): Structure and function of the hepatic form of 11 β-HSD in the squirrel monkey, an animal model of glucocorticoid resistance. Endocrinology 133: 368-375.
7. Kotelevtsev Y V, Iarnieson P M, Best R, Stewart F, Edwards C R W, Seckl J R, Mullins I I (1996): Inactivation of 11 β-HSD type 1 by gene targeting in mice. Endocrinology Res. 22: 791-792.
8. Ricketts M L, Verhaeg J M, Bujalska I, Howie A J, Rainey W E, Stewart P M (1998): Immunohistochemicallocalisation of type 1 11 β-HSD in human tissues. I. Clin. Endoc. Metab. 83: 1325-35.
9. Stewart P M, Sheppard M C (1992): Novel aspects of hormone action: intracellular ligand supply and its control by a series of tissue specific enzymes. Molecular and Cellular Endocrinology 83: C13-C18.
10. Seckl J R, Chapman K E (1997): The 11 β-HSD system, a determinant of glucocorticoid and mineralocorticoid action. Medical and physiological aspects. European I. Biochem. 249: 361-364.
11. Maser E (1998): 11 β-HSD responsible for carbonyl reduction of the tobacco-specific nitrosoamine in mouse lung microsomes. Cancer Res. 58: 2996-3003.
12. Walker B R, Stewart P M, Shackleton C H L, Padfield P L, Edwards C R W (1993): Deficient inactivation of cortisol by 11 β-HSD in essential hypertension. Clin. Endocr. 38:221-227.
13. Daynes R A, Araneo B A (1998): Contrasting effects of glucocorticoids on the capacity of T-cells to produce the growth factors interleukin-2 and interleukin-4. Eur. J. Immunol. 19: 2319-2324.

### FURTHER REFERENCES

Barf, T. et al., (2002), Arylsulfonamidothiazoles as a new class of potential antidiabetic drugs. Discovery of potent and selective inhibitors of the 11β-Hydroxysteroid Dehydrogenase Type 1. J. Med. Chem., 45, 3813-3815.
Matassa, Victor G. et. al. J. Med. Chem.; 33(9); 1990; 2621.
This compound is synthesized in the literature and the NMR spectrum is reported, however the spectrum obtained here differs from that in the literature. Baraldi, Pier Giovanni et. al.; Bioorg. & Med. Chem. Lett.; 10; 2002, 1611.
Horaguchi, Takaaki; Matsuda, Shinichi; Tanemura, Kiyoshi; Suzuki, Tsuneo. J. Heterocyclic Chem.; 24; 1987; 965.
Plé, Patrick A., Marnett, Lawrence J.; J. Heterocyclic Chem.; 25; 1988; 1271.
Rao, U. and Balasubramanian, K. K.; Tetrahedron Lett.; 24; 1983; 5023.
Bordwell, F. G. and Stange, Hugo; J. Amer. Chem. Soc.; 77; 1955; 5939.
Elderfield, Robert C.; Williamson, Thurmond A.; Gensler, Walter J.; Kremer, Chester B.; J. Org. Chem.; 12; 1947; 405.
For 6-nitro-2,3-dimethylquinoxaline see: Barluenga, Jose; Aznar, Fernando; Liz, Ramon; Cabal, Maria-Paz; Synthesis; 3; 1985; 313., then for 6-amino-2,3-dimethylquinoxaline: Salon, Jozef; Milata, Viktor; Pronayova, Nadezda; Lesko, Jan; Collect. Czech. Chem. Commun.; 66; 11; 2001; 1691.
Klicnar, J.; and Kosek, F.; Collect. Czech. Chem. Commun.; 30; 1965; 3102.

Gloster, Daniel F.; Cincotta, Louis; Foley, James W.; J. Heterocyclic Chem.; 36; 1999; 25.
The same reduction was carried out using SnCl₂ by: Case et al.; J. Amer. Chem. Soc.; 81; 1959; 6297.
Modified procedure from similar compound described in U.S. Pat. No. 6,355,796 (Example 20)
Hollfelder, F.; Kirby, A. J.; Tawfik, D. S.; Kikuchi, K.; Hilvert, D.; J. Amer. Chem. Soc.; 122 (6); 2000; 1022-1029
Fujimoto, M.; Okabe, K.; Chem. Pharm. Bull.; 10; 1962; 572-575.
Kawamura, T.; Yagi, N.; Sugawara, H.; Yamahata, K.; Takada, M.;
Chem. Pharm. Bull.; 28; 1; 1980; 268-276.
Stewart, P. M. and Mason, J. I., (1995), Cortisol to cortisone: Glucocorticoid to mineralocortcoid. Steriods, 60, 143-146.
Escher, G. et al., (1995), Furosemide inhibits 11β-Hydroxysteroid Dehydrogenase in vitro and in vivo. Endocrinology, 136, 1759-1765.
Hult, M. et. al., (1998), Selective inhibition of human type 1 11β-hydroxysteroid dehydrogenase by synthetic steroids and xenobiotics. FEBS Letters, 441, 25-28.
Diederich S, Grossmann C, Hanke B, Quinkler M, Herrmann M, Bahr V, Oelkers W (2000): In the search for specific inhibitors of human 11 β-HSD: chenodeoxycholic acid selectively inhibits 11 β-HSD type 1. Europ. J. Endocrin. 142: 200-207.

1. A compound of formula
R₁—CO—X—Y—Z—R₂, or a pharmaceutically acceptable salt thereof,
wherein
X and Z are each optional groups that are, independently, saturated or unsaturated carbon chains having a length of 1 to 3 carbons
Y is SO, S, SO₂, CH═CH, CH₂CH₂ or O
R₁ is 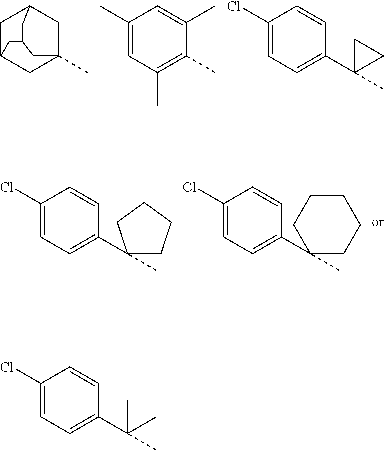
wherein [symbol] denotes the point of attachment
R₂ is a heteroaryl group comprising an optionally substituted 5 or 6 membered ring, which ring contains only carbon and at least one nitrogen, or contains only carbon, and at least two nitrogens and at least one sulfur; and
wherein
(i) when R₁ is 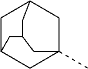
and —CO—X—Y—Z— is CO—CH₂—SO, CO—CH₇—S, or CO—CH₂—SO₂, R₂ is other than 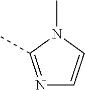
and
(ii) when R₁ is 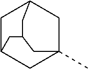
and —CO—X—Y—Z— is —CO—CH₂—O—, R₂ is other than

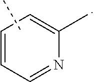

2. A compound according to claim 1, or a pharmaceutically acceptable salt thereof, wherein R₁ is

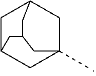

3. A compound according to claim 1, or a pharmaceutically acceptable salt thereof, wherein R₁ is

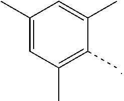

4. A compound according to claim 1, or a pharmaceutically acceptable salt thereof, wherein R₁ is

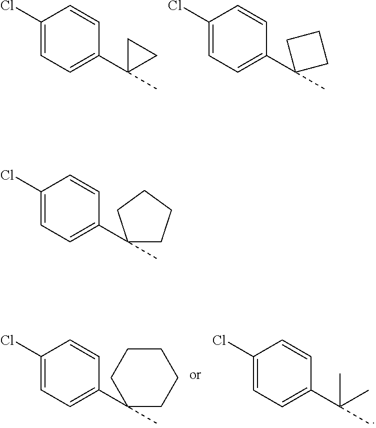

5. A compound according to claim 1 of formula R₁—CO—X—Y—Z—R₂, or a pharmaceutically acceptable salt thereof,
wherein X and Z are independently saturated or unsaturated carbon chains having a length of 1 to 3 carbons, and
Y is SO, S, SO₂, CH═CH, CH₂CH₂ or O.

6. A compound according to claim 1 of formula R₁—CO—X—Y—R₂, or a pharmaceutically acceptable salt thereof,
wherein X is saturated or unsaturated carbon chains having a length of 1 to 3 carbons, and
Y is SO, S, SO₂, CH═CH, CH₂CH₂ or O.

7. A compound according to claim 1 of formula R₁—CO—Y—Z—R₂, or a pharmaceutically acceptable salt thereof,
wherein Z is saturated or unsaturated carbon chains having a length of 1 to 3 carbons, and
Y is SO, S, SO₂, CH═CH, CH₂CH₂ or O.

8. A compound according to claim 1 of formula R₁—CO—Y—R₂, or a pharmaceutically acceptable salt thereof,
wherein Y is SO, S, SO₂, CH═CH, CH₂CH₂ or O.

9. A compound according to claim 1, or a pharmaceutically acceptable salt thereof, wherein X is C₁₋₃ alkylene.

10. A compound according to claim 1, or a pharmaceutically acceptable salt thereof, wherein X is CH₂ or C(CH₃)₂.

11. A compound according to claim 1, or a pharmaceutically acceptable salt thereof, wherein Z is C₁₋₃ alkylene.

12. A compound according to claim 1, or a pharmaceutically acceptable salt thereof, wherein Z is CH₂.

13. A compound according to claim 1, or a pharmaceutically acceptable salt thereof, wherein X is C₁₋₃ alkylene and Z is C₁₋₃ alkylene.

14. A compound according to claim 1, or a pharmaceutically acceptable salt thereof, wherein X is CH₂ or C(CH₃)₂ and Z is CH₂.

15. A compound according to claim 1 of formula R₁—CO—X—Y—Z—R₂, or a pharmaceutically acceptable salt thereof,
wherein
X is C₁₋₃ alkylene;
Z is an optional C₁₋₃ alkylene group; and
Y is SO, S, or SO₂.

16. A compound according to claim 15, or a pharmaceutically acceptable salt thereof, wherein X is CH₂ or C(CH₃)₂ and Z is an optional CH₂ group.

17. A compound according to claim 1 of formula R₁—CO—X—O—Z—R₂, or a pharmaceutically acceptable salt thereof,
wherein
X is C₁₋₃alkylene; and
Z is an optional C₁₋₃alkylene group.

18. A compound according to claim 17, or a pharmaceutically acceptable salt thereof, wherein X is CH₂ and Z is an optional CH₂ group.

19. A compound according to claim 1 of formula R₁—CO—Y—R₂, or a pharmaceutically acceptable salt thereof,
wherein
Y is CH═CH or CH₂CH₂.

20. A compound according to claim 1, or a pharmaceutically acceptable salt thereof, wherein —CO—X—Y—Z— is COCH₂S, COCH₂SO, COCH₂SO₂, COCH₂SCH₂, COCH₂SOCH₂, COCH₂SO₂CH₂, COC(CH₃)₂SO, COCH₂O, COCH₂OCH₂, COCH═CH or COCH₂CH₂.

21. A compound according to claim 1, or a pharmaceutically acceptable salt thereof, wherein R₂ is a heteroaryl group comprising an optionally substituted 5 or 6 membered ring, which ring contains only carbon and at least one nitrogen.

22. A compound according to claim 1, or a pharmaceutically acceptable salt thereof, wherein R₂ is a heteroaryl group comprising an optionally substituted 5 membered ring which ring contains only carbon and at least one nitrogen.

23. A compound according to claim 1, or a pharmaceutically acceptable salt thereof, wherein R₂ is a heteroaryl group comprising an optionally substituted 6 membered ring which ring contains only carbon and at least one nitrogen.

24. A compound according to claim 1, or a pharmaceutically acceptable salt thereof, wherein the R₂ optional substituents together form a further ring fused to the 5 or 6 membered heteroaryl ring.

25. A compound according to claim 1, or a pharmaceutically acceptable salt thereof, wherein R₂ is a heteroaryl group comprising an optionally substituted 5 or 6 membered ring, which ring or contains only carbon, and at least two nitrogens and at least one sulfur.

26. A compound according to claim 1, or a pharmaceutically acceptable salt thereof, wherein the R₂ group is an optionally substituted 5 or 6 membered heteroaryl ring of the formula

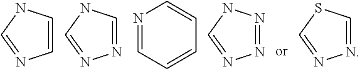

27. A compound according to claim 1, or a pharmaceutically acceptable salt thereof, wherein the R₂ group is an optionally substituted 5 or 6 membered heteroaryl ring of the formula 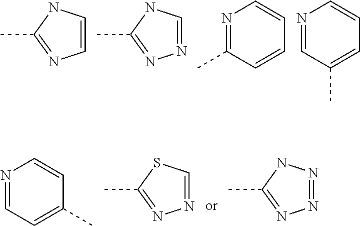
wherein [symbol] denotes the point of attachment.

[symbol]

28. A compound according to claim 1, or a pharmaceutically acceptable salt thereof, wherein the R₂ group is optionally substituted with hydrocarbyl groups, halogens, hydroxyl, carbonyl, amines, and amides.

29. A compound according to claim 1, or a pharmaceutically acceptable salt thereof, wherein each optional substituent of the R₂ group is independently an oxy group; an ether group; a thioether group; an aryl group; an aryl group substituted with one or more alkyl groups or halogens; an alkyl group; an alkoxy group; a haloalkyl group; a halogen; an amide group; or a carbonyl group; or two R₂ groups together form an aryl group fused to the 5 or 6 membered heteroaryl ring.

30. A compound according to claim 1, or a pharmaceutically acceptable salt thereof, wherein each optional substituent of the R₂ group is independently a C₁₋₅ alkyl group; a C₃₋₆ cycloalkyl group; an ether group containing from 1 to 5 carbons; a thioether group containing from 1 to 5 carbons; a C₁₋₅ alkoxy group; a C₁₋₅ haloalkyl group; a halogen; an oxy group; an amine; a phenyl group; a furan group; a thiophene group; a —(C₁₋₅ alkyl)-phenyl group substituted by one or more halogens; an amide group; an alkyl amide group; a dialkyl amide group; or an acylamide group; or two R₂ groups together form a phenyl group fused to the 5 or 6 membered heteroaryl ring.

31. A compound according to claim 1, or a pharmaceutically acceptable salt thereof, wherein each optional substituent of the R₂ group is independently methyl, methoxy, oxy, chloro, CH(CH₃)₂, —S-Me, —CH₂—O-Me, CF₃, NMe₂, COOH, C═ONH₂, C═ONHMe, C═ONMe₂, C═ONHCH₂CH₃, —NH₂, phenyl, furan, thiophene, —NH—C═OMe, —NH—C═O-cyclopropane, cyclopropane, or CH₂-4-chlorophenyl, or together form a phenyl group fused to the 5 or 6 membered heteroaryl ring.

32. A compound according to claim 1, or a pharmaceutically acceptable salt thereof, wherein R₂ is

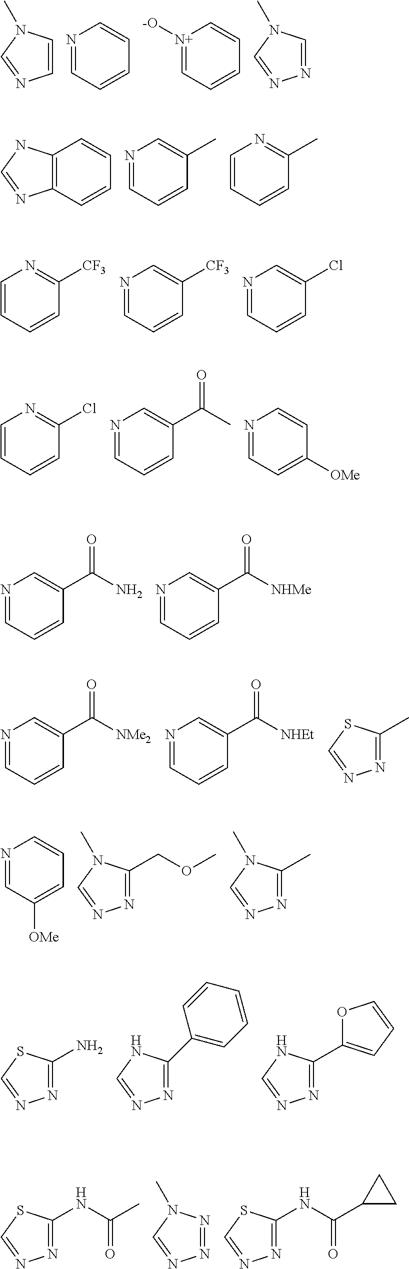

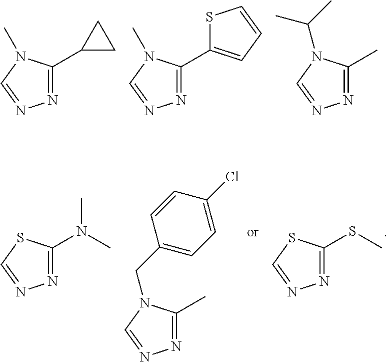

33. A compound according to claim 1, or a pharmaceutically acceptable salt thereof, wherein R₂ is 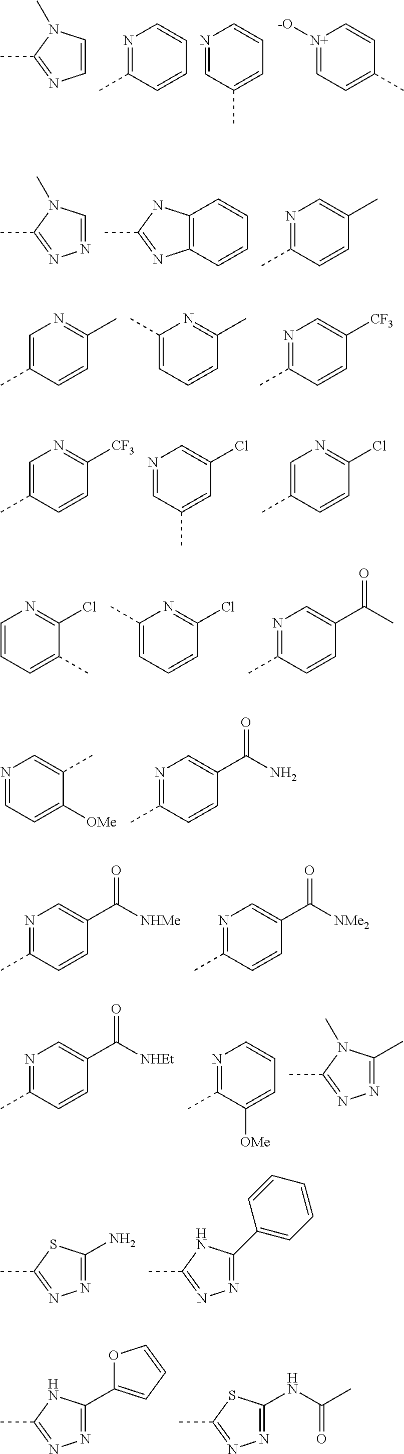 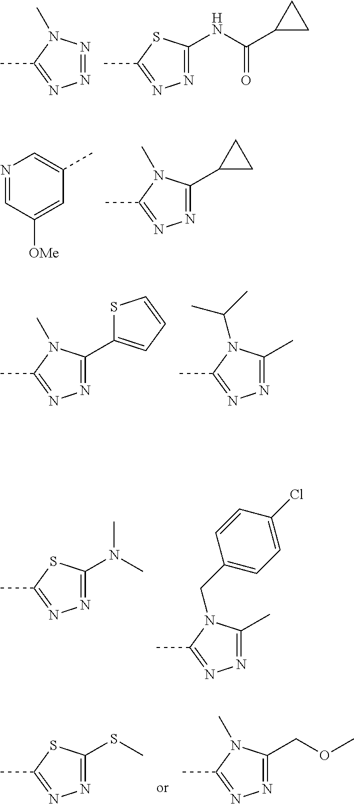
wherein [symbol] denotes the point of attachment.

[symbol]

34. A compound according to claim 1 which is 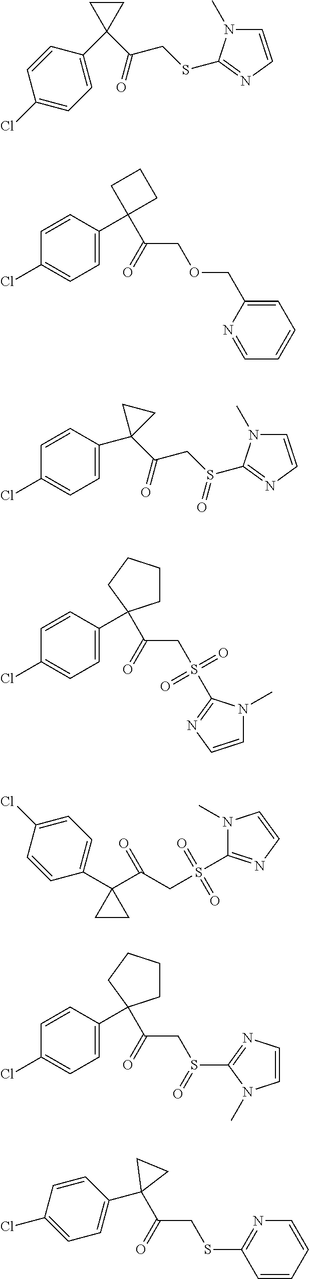 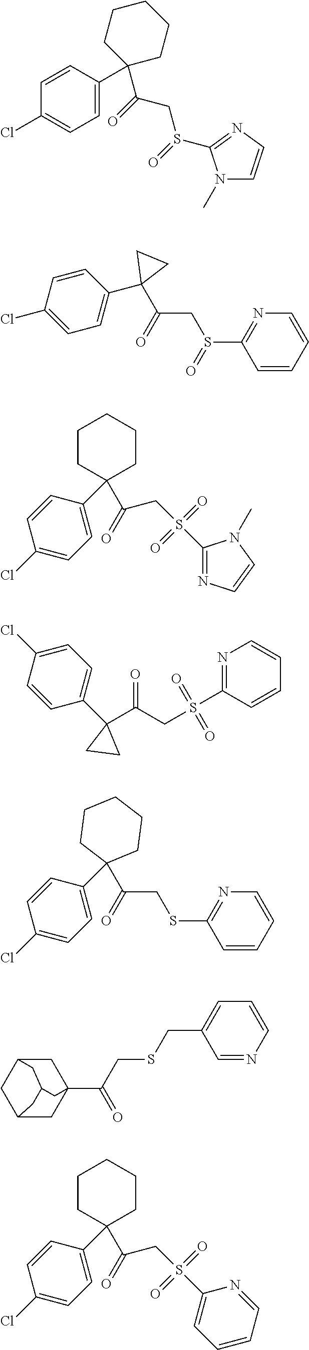 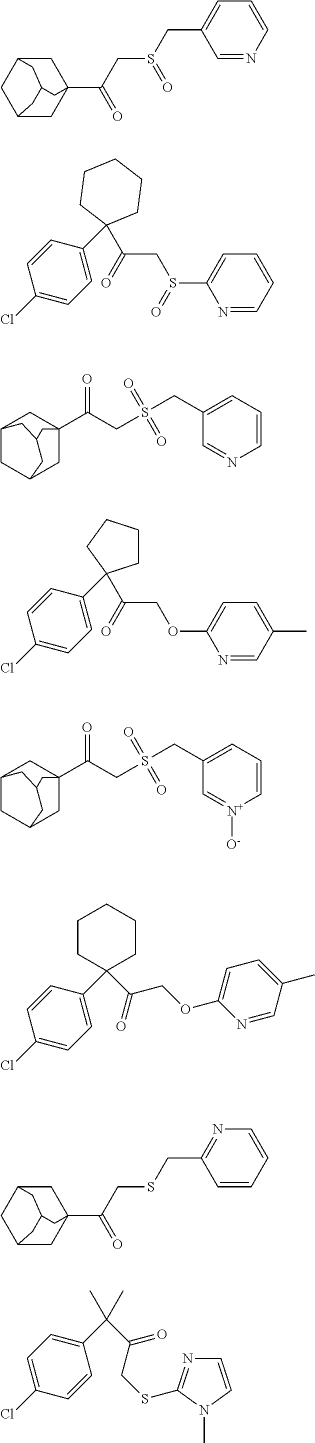 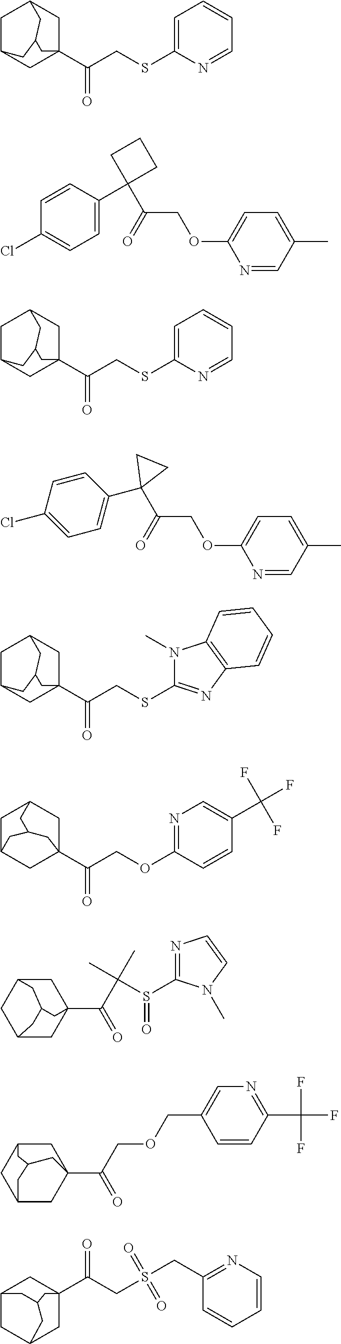 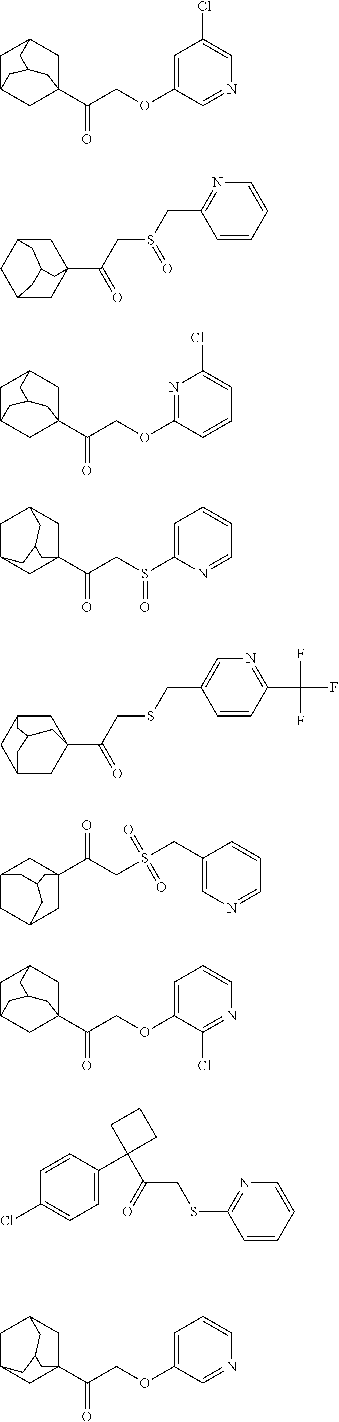 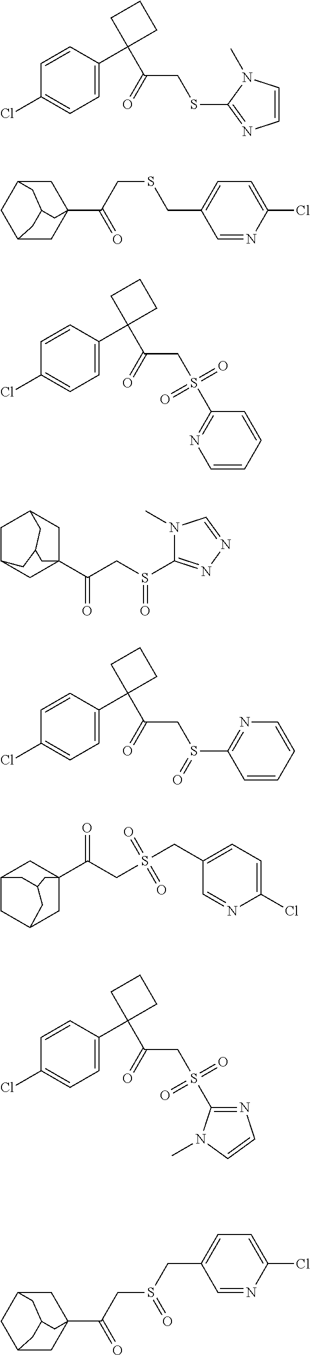 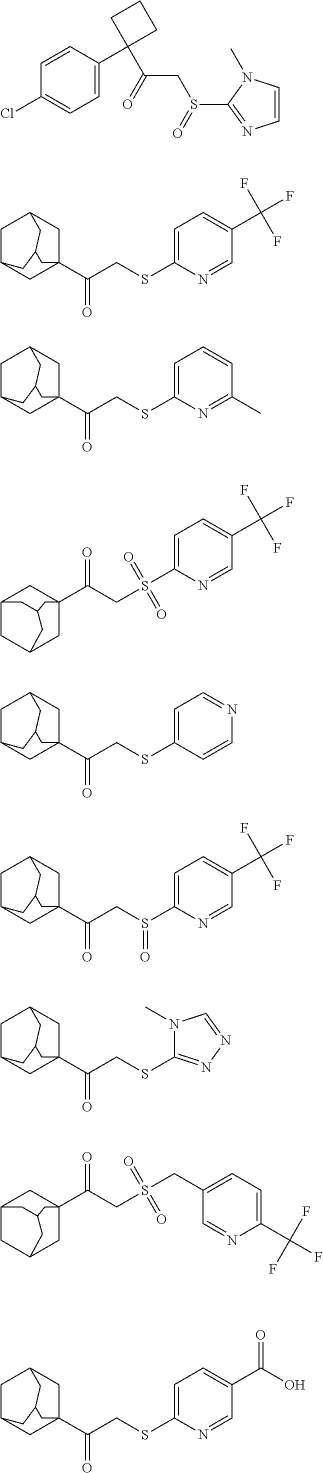 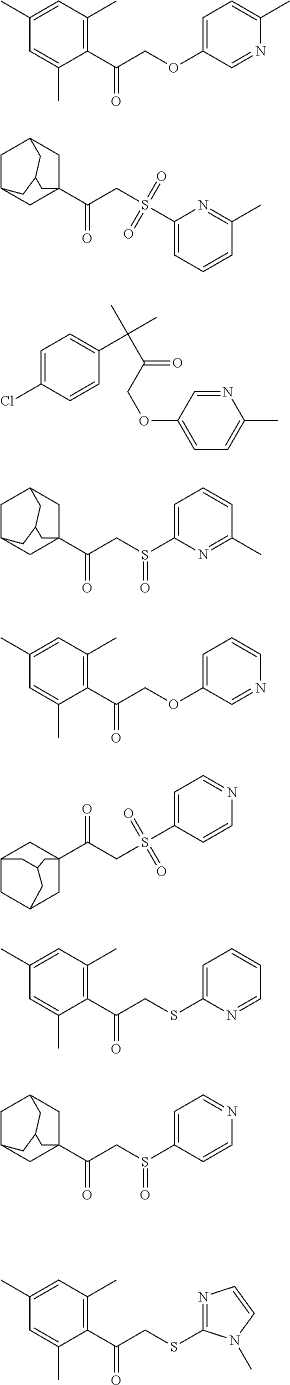 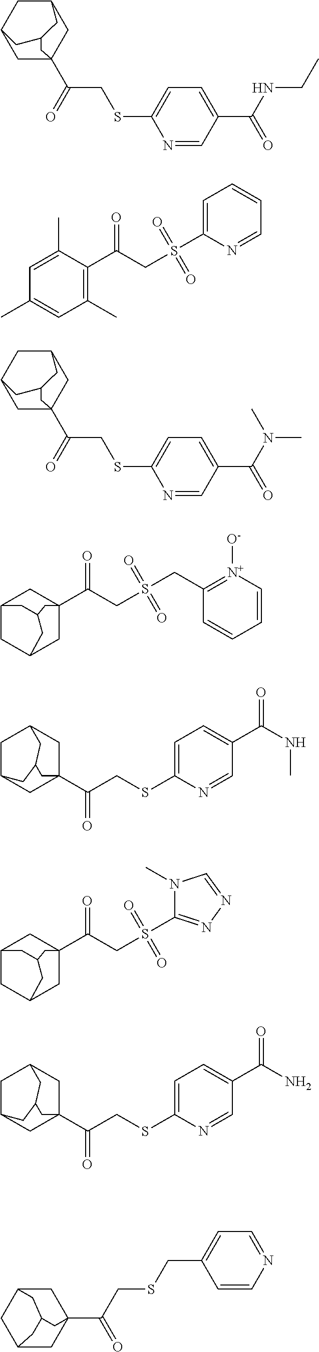 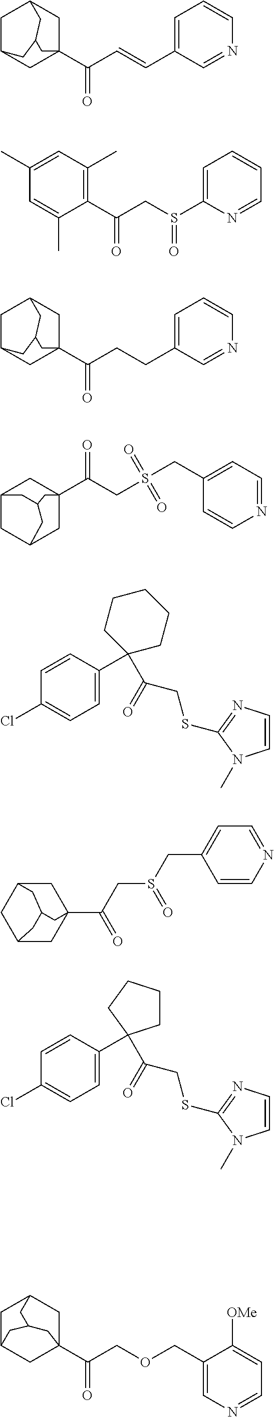 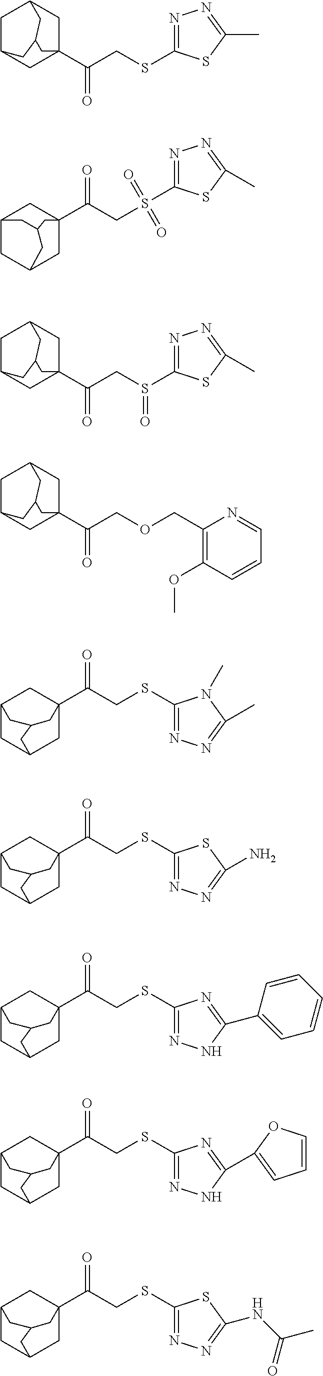 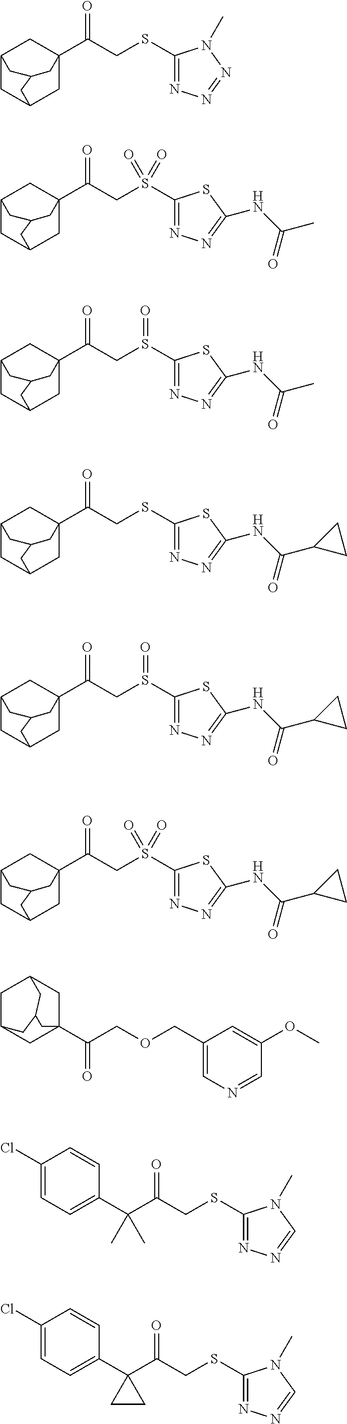 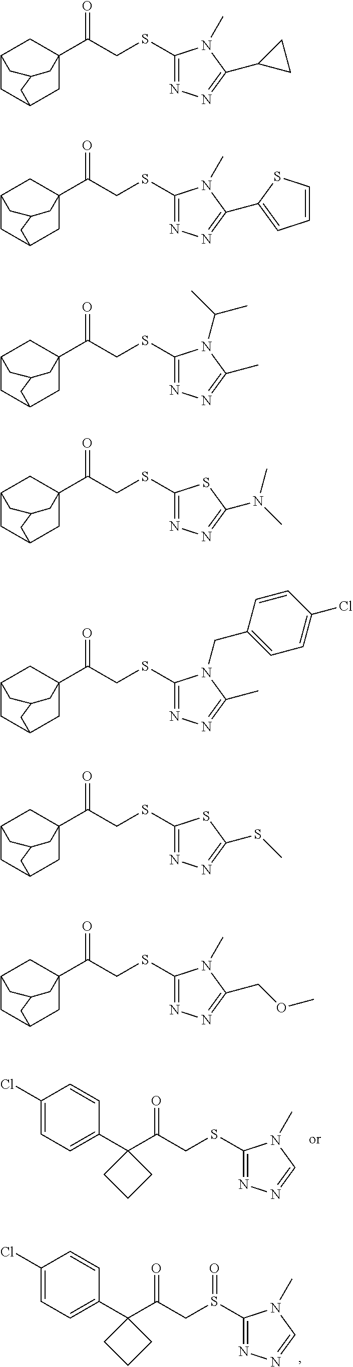
or a pharmaceutically acceptable salt thereof.

35. A pharmaceutical composition comprising a compound according to claim 1, or a pharmaceutically acceptable salt thereof, and a pharmaceutically acceptable carrier, diluent, excipient or adjuvant.

36. (canceled)

37. A method of treating or preventing a condition or disease associated with 11β-HSD, comprising administering a compound according to claim 1, or a pharmaceutically acceptable salt thereof, to a subject in need thereof.

38. (canceled)

39. The method of claim 37, wherein the condition or disease is metabolic disorders; cardiovascular disorders; glaucoma; inflammatory disorders; immune disorders; bone disorders; cancer; intra-uterine growth retardation; apparent mineralocorticoid excess syndrome (AME); polycystic ovary syndrome (PCOS); hirsutism; acne; oligo- or amenorrhea; adrenal cortical adenoma and carcinoma; Cushing's syndrome; pituitary tumours; invasive carcinomas; breast cancer; or endometrial cancer.

40. A method of treating or preventing a condition or disease associated with adverse 11β-HSD levels comprising administering a compound according to claim 1, or a pharmaceutically acceptable salt thereof, to a subject in need thereof.

41. (canceled)

42. A method for modulating 11β-HSD activity, comprising administering a compound according to claim 1, or a pharmaceutically acceptable salt thereof, to a subject in need thereof.

43. (canceled)

44. A method for inhibiting 11β-HSD activity, comprising administering a compound according to claim 1, or a pharmaceutically acceptable salt thereof, to a subject in need thereof.

45-49. (canceled)

50. A composition comprising the compound according to claim 1, or a pharmaceutically acceptable salt thereof, in an amount effective to treat or prevent a disease associated with 11β-HSD.

51. The method of claim 39, wherein the metabolic disorder is diabetes or obesity.

52. The method of claim 39, wherein the cardiovascular disorder is hypertension.

53. The method of claim 39, wherein the inflammatory disorder is arthritis or asthma.

54. The method of claim 39, wherein the bone disorder is osteoporosis.
